SEQUENCE LISTING (3 sequences)

SEQ ID NO: 1 - DNA, 65 bp, synthetic construct
  source 1..65: mol_type = other DNA
gatccccgaagagaggttcagctaaattcaagagatttagctgaacctctcttcttttttggaaa

SEQ ID NO: 2 - DNA, 37 bp, synthetic construct
  source 1..37: mol_type = other DNA
ttctggcgggctcgagcgtcgacatggaacagaaact

SEQ ID NO: 3 - DNA, 36 bp, synthetic construct
  source 1..36: mol_type = other DNA
taccgagcccgaattgaattcctcgaggccacgaag

(19) United States
(12) Patent Application Publication
(10) Pub. No.: US 2025/0312462 A1
(43) Pub. Date: Oct. 9, 2025

(54) PAK1 DEGRADERS AND METHODS OF USE THEREOF

(71) Applicants: INSTITUTE FOR CANCER RESEARCH D/B/A THE RESEARCH INSTITUTE OF FOX CHASE CANCER CENTER, Philadelphia, PA (US); DANA-FARBER CANCER INSTITUTE, INC., Boston, MA (US); THE BOARD OF TRUSTEES OF THE LELAND STANFORD JUNIOR UNIVERSITY, Stanford, CA (US)

(72) Inventors: Nathanael S. GRAY, Stanford, CA (US); Brian GROENDYKE, Boston, MA (US); Sean TOENJES, Stanford, CA (US); Jonathan CHERNOFF, Philadelphia, PA (US)

(73) Assignees: INSTITUTE FOR CANCER RESEARCH D/B/A THE RESEARCH INSTITUTE OF FOX CHASE CANCER CENTER, Philadelphia, PA (US); DANA-FARBER CANCER INSTITUTE, INC., Boston, MA (US); THE BOARD OF TRUSTEES OF THE LELAND STANFORD JUNIOR UNIVERSITY, Stanford, CA (US)

(21) Appl. No.: 18/865,225

(22) PCT Filed: May 12, 2023
(86) PCT No.: PCT/US2023/066943

§ 371 Date: Nov. 12, 2024

Related U.S. Application Data
(60) Provisional application No. 63/341,930, filed on May 13, 2022.
(60) Provisional application No. 63/341,887, filed on May 13, 2022.
(60) Provisional application No. 63/379,504, filed on Oct. 14, 2022.

(51) Int. Cl. A61K 47/55; A61K 47/54; A61P 35/00

(52) U.S. Cl. CPC A61K 47/55; A61K 47/545; A61P 35/00

(57) ABSTRACT

PAK1 degraders and methods of use thereof are disclosed.

### RELATED APPLICATIONS

This application claims the benefit of priority under 35 U.S.C. § 119(e) to U.S. Provisional Application No. 63/341,887, filed May 13, 2022, U.S. Provisional Application No. 63/341,930, filed May 13, 2022, and U.S. Provisional Application No. 63/379,504, filed Oct. 14, 2022, each of which are incorporated herein by reference in their entireties.

### GOVERNMENT LICENSE RIGHTS

This invention was made with government support under contract Nos. R01-CA227184, R01-CA218278, and R01-CA148805 awarded by the National Institutes of Health. The government has certain rights in the invention.

### FIELD OF THE INVENTION

The present invention relates to the field of degraders. More specifically, the invention provides compounds which target and degrade PAK1.

### SEQUENCE LISTING

The instant application contains a Sequence Listing which has been submitted electronically in XML format and is hereby incorporated by reference in its entirety. Said XML copy, created on May 4, 2023, is named 60293-501001WO-ST26.xml and is 4 KB bytes in size.

### BACKGROUND OF THE INVENTION

Several publications and patent documents are cited throughout the specification in order to describe the state of the art to which this invention pertains. Each of these citations is incorporated herein by reference as though set forth in full.

In 2015, a dibenzodiazepine-based small molecule inhibitor, NVS-PAK1-1, was described that showed excellent specificity for PAKs over other kinases and a ˜50× selectivity for PAK1 over PAK2 (Karpov, et al. (2015) ACS Med. Chem. Lett., 6:776-781). Given the extensive similarity between the catalytic domains of PAK1 and PAK2 (93% identical), this selectivity was unexpected, as was the co-crystal structure which revealed that the molecule occupied a space in the catalytic cleft underneath the αC-helix rather than in the hinge region of the ATP binding pocket. However, NVS-PAK1-1 has a short half-life in rat liver microsomes and is metabolized in vivo by the cytochrome P450 system (Hawley, et al. (2021) Human Mol. Genet., 30(17):1607-1617; Karpov, et al. (2015) ACS Med. Chem. Lett., 6:776-781).

Another class of small molecule inhibitors has displayed a similar selectivity for PAK1 over PAK2, which is achieved by binding to the less conserved p21-binding domain at the N-terminus of PAK1, as opposed to the highly conserved kinase domain (Kim, et al. (2016) Exp. Mol. Med., 48:e229). However, these compounds are only effective at micromolar doses.

In view of the foregoing, there is a clear need for improved selective inhibition of PAK1.

### SUMMARY OF THE INVENTION

In accordance with the present invention, PAK1 degraders are provided. In some embodiments, the PAK1 degrader is a proteolysis-targeting chimeric molecule (PROTAC). In some embodiments, the PAK1 degrader selectively degrades PAK1 over other PAKs, particularly PAK2. In some embodiments, the PAK1 degrader comprises NVS-PAK1-1 linked to a degron, optionally via a linker (e.g., an alkyl, a hydrocarbon, or polyethylene glycol). In some embodiments, the degron is linked to the NVS-PAK-1 at the isopropyl urea. In some embodiments, the degron is linked to NVS-PAK-1 at the carbon after removal of —NH(isopropyl) from the isopropyl urea. In some embodiments, the degron is linked to NVS-PAK-1 at the nitrogen after removal of the isopropyl from the isopropyl urea. In some embodiments, the degron is a ligand for an E3 ubiquitin ligase such as CRBN. In some embodiments, the PAK1 degrader is BJG-05-039 or a pharmaceutically acceptable salt or stereoisomer thereof.

Some embodiments of the present invention are directed to a compound having a structure represented by formula (I):

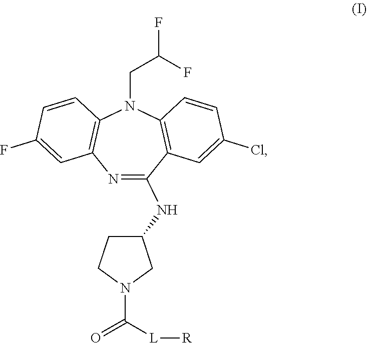

wherein L is a linker and R is a degron,
the linker is a chemical moiety that covalently attaches the carbonyl carbon to the degron; and the degron is a ligand for an E3 ubiquitin ligase,
or a pharmaceutically acceptable salt or stereoisomer thereof. In some embodiments, the compound of formula I is of formula II:

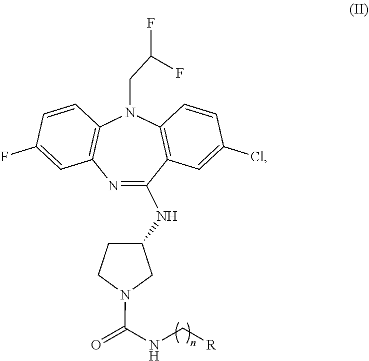

wherein R is a degron and n=1 to 15, or a pharmaceutically acceptable salt or stereoisomer thereof.

Other embodiments of the present invention are directed to a compound having a structure represented by formula (III):

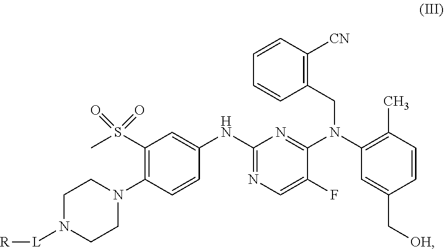

wherein L is a linker and R is a degron,
the linker is a chemical moiety that covalently attaches the nitrogen to the degron; and
the degron is a ligand for an E3 ubiquitin ligase,
or a pharmaceutically acceptable salt or stereoisomer thereof.

Another aspect of the present invention is directed to a pharmaceutical composition containing a therapeutically effective amount of a compound of formula (I or III) or a pharmaceutically acceptable salt or stereoisomer thereof, and a pharmaceutically acceptable carrier.

In another aspect of the present invention, methods of making the compounds are provided.

In accordance with another aspect of the instant invention, methods of treating, inhibiting, and/or preventing a disease or disorder associated with the aberrant overexpression; aberrant increased activity of PAK1; and/or amplification of the PAK1 gene are provided. In some embodiments, the overexpression of PAK1; increased activity (e.g., kinase activity) of PAK1; and/or amplification of the PAK1 gene are in comparison to wild-type, healthy, and/or normal (e.g., non-diseased) cells. In some embodiments, the PAK1 associated disease or disorder is cancer. In some embodiments, the PAK1 associated disease or disorder is neurofibromatosis type 1 (NF1) or neurofibromatosis type 2 (NF2). In some embodiments, the method further comprising administering another therapy to the subject (e.g., for treating NF1 or NF2 or treating cancer).

### BRIEF DESCRIPTION OF THE DRAWINGS

FIG. 1A shows the specificity of NVS-PAK1-1. In vitro kinase assays were performed using recombinant PAK1 and PAK2, using a library of small molecule protein kinase inhibitors. FIG. 1B shows the chemical structure of BJG-05-039. FIG. 1C is a series of Western blots showing the degradation of PAK1 in Panc1 cells by the NVS-PAK1-1-based (allosteric) degraders BJG-05-014, BJG-05-027, and BJG-05-039. Quantification is shown below in the bar graph. FIG. 1D is a series of Western blots showing PAK1, PAK2, and GAPDH in Panc1 cells treated with the indicated amounts of the NVS-PAK1-1-based degrader BJG-05-039 and ATP-competitive degraders BJG-05-093, BJG-05-094, and BJG-05-095.

FIG. 2A-FIG. 2D show that BJG-05-039 induces selective degradation of PAK1 dependent on Cereblon (CRBN), neddylation, and the proteosome. FIG. 2A is a series of Western blots showing the effects of BJG-05-039, BJG-05-098, and NVS-PAK1-1 on PAK1 and PAK2 levels. MCF7 and OVCAR3 cells were treated with increasing concentrations of the indicated compounds for 24 hours and protein lysates were analyzed by immunoblot. Asterisk indicates PAK1 signal on immunoblot. FIG. 2B is a Western blot showing time course of PAK1 degradation. FIG. 2C is a Western blot showing the effect of Bortezomib and Lenalidomide, respectively, on degrader capacity of BJG-05-039. FIG. 2D is a bar graph showing the quantitation of PAK1 expression by luminescence assay. HEK293 cells stably expressing near-endogenous levels of Nluc-PAK1 were treated with the indicated concentrations of BJG-05-039. 24 hours post treatment the cells were lysed an analyzed for luciferase activity. FIG. 2E is a scatterplot showing the effect of BJG-05-039 on the proteome. Scatterplot depicts the change in relative protein abundance of MOLT cells treated with BJG-05-039 (5 hours, 1 μM) compared with DMSO vehicle control-treated cells. Protein abundance measurements were made using tandem mass spectrometry and significant changes were assessed by moderated t test as implemented in the limma package (Ritchie, et al. (2015) Nucleic Acids Res., 43(7):e47). The log₂ fold change (log₂ FC) is shown on the y-axis and negative log₁₀ p value (−log₁₀ p value) on the x-axis for three independent biological replicates of each treatment.

FIG. 3A-FIG. 3C show that the PAK1 degrader potently suppresses proliferative signals. FIG. 3A is a series of Western blots showing OVCAR3 and MCF7 cells that were treated for 24 hours with DMSO, 10 nM BJG-05-039, or NVS-PAK1-1 as indicated. MEK and ERK phosphorylation was assessed by immunoblot with the indicated phosphorylation-specific antibodies. FIG. 3B is a Western blot showing MCF7 cells that were stably transduced with a doxycycline-regulated shRNA against PAK1. shRNA expression was induced by the indicated amounts of doxycycline and immunoblots were performed using cell lysates 24 hours post doxycycline addition. FIG. 3C is a Western blot showing MCF7 cells that were treated with vehicle or 1 mg/mL doxycycline. Immunoblots were performed using cell lysates 24 hours post doxycycline addition.

FIG. 4A-FIG. 4E show that the PAK1 degrader selectively suppresses proliferation of PAK1-dependent cells. PAK1-dependent (MCF7 and OVCAR3) and PAK2-dependent (OMM1 and HeyA8) cells were treated for 96 hours with varying concentrations of BJG-05-039 (FIG. 4A), BJG-05-098 (FIG. 4B), NVS-PAK1-1 with PAK1 shRNA (FIG. 4C) or lenalidomide (FIG. 4D). Cell proliferation was assessed by MTT assay. FIG. 4E is a graph showing the effect of reducing PAK1 expression on the potency of NVS-PAK1-1 was assessed by treating control MCF7 cells or MCF7 cells in which PAK1 expression was reduced ˜50% via induction of a PAK1-specific shRNA at 0.5 mg/mL doxycycline.

FIG. 5A and FIG. 5B are synthetic schemes for ATP-competitive degraders.

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

The singular forms “a,” “an,” and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the invention.

The term “isolated” is not meant to exclude artificial or synthetic mixtures with other compounds or materials, or the presence of impurities that do not interfere with the fundamental activity, and that may be present, for example, due to incomplete purification, or the addition of stabilizers.

The term “treat” as used herein refers to any type of treatment that imparts a benefit to a patient suffering from an injury, including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury, resulting in a decrease in the probability that the subject will develop conditions associated with a disease or disorder (e.g., cancer).

With respect to compounds of the present invention, and to the extent the following terms are used herein to further describe them, the following definitions apply.

As used herein, the term “alkyl” refers to an optionally substituted saturated, branched or linear hydrocarbon radical group. In some embodiments, the alkyl radical is a C₁-C₆ group. In some embodiments, and to the extent not disclosed otherwise for any one or more groups of the compounds of formula (I-III), the alkyl radical is a C₀-C₆, C₀-C₅, C₀-C₃, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. In some embodiments, an alkyl group is a C₁-C₃ alkyl group. In some embodiments, an alkyl group is a C₁-C₂ alkyl group. In some embodiments, an alkyl group is a methyl group. “Substituted alkyl,” as used herein, refers to an alkyl group that is substituted with one or more functional groups such as oxo, C₁-C₃ alkyl (e.g., methyl), C₂-C₄ alkenyl, C₁-C₃ alkoxy (e.g., methoxy), C₁-C₃ monoalkylamino (—NH(alkyl)), C₁-C₃ dialkylamino (—N(alkyl)₂), halogen, —OH, —SH, —NH₂, —COOH, —CN, and/or —NO₂.

As used herein, the term “alkylene” refers to a straight or branched divalent hydrocarbon chain linking the rest of the molecule to a radical group, consisting solely of carbon and hydrogen, containing no unsaturation and having from one to 15 carbon atoms, for example, methylene, ethylene, propylene, n-butylene, and the like. The alkylene chain may be attached to the rest of the molecule through a single bond and to the radical group through a single bond. In some embodiments, the alkylene group contains one to 15 carbon atoms (C₁-C₁₅ alkylene). In some embodiments, the alkylene group contains one to 12 carbon atoms (C₁-C₁₂ alkylene). In some embodiments, the alkylene group contains one to 10 carbon atoms (C₁-C₁₀ alkylene). In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In some embodiments, the alkenyl radical is a C₂-C₁₅ group. In some embodiments, and to the extent not disclosed otherwise for any one or more groups of the compounds of formula (I-III), the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In some embodiments, the alkynyl radical is a C₂-C₁₅ group. In some embodiments, and to the extent not disclosed otherwise for any one or more groups of the compounds of formula (I-III), the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. In some embodiments, the alkoxyl group is methoxy, ethoxy, propyloxy, or tert-butoxy. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 12 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 10 carbon atoms (C₃-C₁₀). In one embodiment, carbocyclyl includes 3 to 6 carbon atoms (C₃-C₆). In one embodiment, carbocyclyl includes 5 to 6 carbon atoms (C₅-C₆). In some embodiments, carbocyclyl, as a bicycle, includes C₆-C₁₀. In another embodiment, carbocyclyl, as a spiro system, includes C₅-C₁₁. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and phenyl; bicyclic carbocyclyls having 7 to 11 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocyclyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-12 carbon atoms. In another embodiment, aryl includes groups having 6-10 carbon atoms. Examples of aryl groups include phenyl, naphthyl, biphenyl, 1,2,3,4-tetrahydronaphthalenyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, 4, or 5) carbon atoms have been replaced with a heteroatom or heteroatom-containing group (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3- to 12-membered heterocyclyl ring system. In some embodiments, a heterocyclyl refers to a saturated ring system, such as a 3- to 12-membered saturated heterocyclyl ring system. In some embodiments, a heterocyclyl refers to a heteroaryl ring system, such as a 5- to 12-membered heteroaryl ring system. The term heterocyclyl also includes C₂-C₈ heterocycloalkyl, which is a saturated or partially unsaturated mono-, bi-, or spiro-ring system containing 2-8 carbons and one or more (e.g., 1, 2, or 3) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from O, N, and S. In some embodiments, heterocyclyl includes 4- to 6-membered monocycles having one or more heteroatoms selected from O, N, and S. In some embodiments, heterocyclyl includes 3-membered monocycles. In some embodiments, heterocyclyl includes 4-membered monocycles. In some embodiments, heterocyclyl includes 5- to 6-membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be substituted (e.g., methyl, isopropyl) and/or quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl (e.g., thiazol-2-yl), thiadiazolyl (e.g., 1,3,4-thiadiazol-5-yl and 1,2,4-thiadiazol-5-yl), oxazolyl (e.g., oxazol-2-yl), and oxadiazolyl (e.g., 1,3,4-oxadiazol-5-yl and 1,2,4-oxadiazol-5-yl). Example of 5-membered heterocyclyls containing 2 to 4 nitrogen atoms include imidazolyl (e.g., imidazol-2-yl), triazolyl (e.g., 1,3,4-triazol-5-yl, 1,2,3-triazol-5-yl, and 1,2,4-triazol-5-yl), and tetrazolyl (e.g., 1H-tetrazol-5-yl). Representative examples of benzo-fused 5-membered heterocyclyls include benzoxazol-2-yl, benzthiazol-2-yl and benzimidazol-2-yl. Example of 6-membered heterocyclyls containing one to three nitrogen atoms and optionally a sulfur or oxygen atom are pyridyl (e.g., pyrid-2-yl, pyrid-3-yl, and pyrid-4-yl), pyrimidyl (e.g., pyrimid-2-yl and pyrimid-4-yl), triazinyl (e.g., 1,3,4-triazin-2-yl and 1,3,5-triazin-4-yl), pyridazinyl (e.g., pyridazin-3-yl), and pyrazinyl. In some embodiments, a heterocyclic group includes a heterocyclic ring fused to one or more (e.g., 1 or 2) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen atom and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, 1-pyrazolidinyl, 1-imidazolinyl and 1-imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2- or 3-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”)) refers to a monocyclic, bicyclic or tricyclic ring system having 5 to 12 ring atoms, wherein at least one ring is aromatic and contains at least one heteroatom. In one embodiment, heteroaryl includes 5- to 6-membered monocyclic aromatic groups where one or more ring atoms is O, N, or S. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, and 1,2,3-triazol-5-yl. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1 or 2) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s) in the compounds of formula (I-III), any of the groups described herein may be substituted or unsubstituted. To the extent not disclosed otherwise for any particular group(s), representative examples of substituents may include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., substituted C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3- to 12-membered, 5- to 6-membered), substituted heterocyclic (e.g., substituted 3- to 12-membered, 5- to 6-membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

p21-activated kinases (PAKs) have been considered as potential drug targets in a variety of cancers (Dummler, et al. (2009) Cancer Metastasis Rev., 28:51-63; Radu, et al. (2014) Nat. Rev. Cancer 14:13-25; Rane, et al. (2019) Semin. Cancer Biol., 54:40-49; Ye, et al. (2012) Cell Logist., 2:105-116). The PAK family is comprised of two groups: group A (PAK1, -2, and -3) and Group B (PAK4, -5-, and -6) (Jaffer, et al. (2002) Int. J. Biochem. Cell Biol., 34:713-717; Rane, et al. (2014) Small GTPases 5:e28003). The three members of the Group A PAKs are all closely related in sequence and structure, whereas the three Group B PAKs are distinct from the Group A proteins as well as more distantly related to one another. In addition to their structural differences, the six isoforms have distinct though sometimes overlapping expression patterns. For example, PAK1 is primarily expressed in brain, muscle, and blood cells; PAK2 is ubiquitous; and PAK3 is primarily expressed in neuronal cells. Of the Group B PAKs, PAK4 is ubiquitous, PAK5 is expressed mainly in neuronal cells, and PAK6 is expressed in neuronal cells, skin, prostate and testes (Rane, et al. (2019) Semin. Cancer Biol., 54:40-49; Sells, et al. (1997) Curr. Biol., 7:202-210). Genetic loss-of-function analyses in animal models has shown a variety of different phenotypes, ranging from embryonic lethality (PAK2, PAK4), to cognitive dysfunction (PAK3), to minimal effects (PAK1, PAK5, PAK6) (Hofmann, et al. (2004) J. Cell. Sci., 117:4343-4354; Minden, A. (2012) Cell Logist., 2:95-104; Zhao, et al. (2012) Cell Logist., 2:59-68).

As effectors of the small GTPase RAC, the PAK enzymes regulate several key proliferative and survival pathways including the RAF-MEK-ERK, the PI3K-AKT-mTORC, and the β-catenin pathways (Radu, et al. (2014) Nat. Rev. Cancer 14:13-25). While rarely subject to mutational activation, certain PAK isoforms, in particular PAK1 and PAK4, are frequently expressed at high levels in various tumor types due to chromosomal amplifications of their corresponding genes at chromosome 11q13 and 19q13, respectively (Radu, et al. (2014) Nat. Rev. Cancer 14:13-25; Ye, et al. (2012) Cell Logist., 2:105-116), or are activated by mutations in RAC1 (Araiza-Olivera, et al. (2018) Oncogene 37:944-952; Krauthammer, et al. (2012) Nature Genet., 44:1006-1014). Reducing PAK activity, via gene knockouts, RNA interference, or small molecule inhibitors, has been shown to be of benefit in many cell-based and in vivo cancer models (Radu, et al. (2014) Nat. Rev. Cancer 14:13-25).

These factors led to the development of various PAK inhibitors for cancer therapy (Licciulli, et al. (2013) J. Biol. Chem., 288:29105-29114; Murray, et al. (2010) Proc. Natl. Acad. Sci., 107:9446-9451; Ndubaku, et al. (2015) ACS Med. Chem. Lett., 6:1241-1246; Ong, et al. (2015) Breast Cancer Res., 17:59; Rudolph, et al. (2016) J. Med. Chem., 59:5520-5541). One of these, Pfizer's pan-PAK inhibitor PF3758309, was evaluated in a human Phase 1 clinical trial, but was withdrawn due to a combination of poor pharmaceutical properties and toxicity (Radu, et al. (2014) Nat. Rev. Cancer 14:13-25). Afraxis and Genentech described a series of increasingly specific Group A PAK inhibitors, which were found to be effective in preclinical models of NF2, KRAS-driven squamous cell carcinoma, and HER2-driven breast cancer (Arias-Romero, et al. (2013) Cancer Res., 73:3671-3682; Chow, et al. (2015) Oncotarget 6:1981-1994; Chow, et al. (2012) Cancer Res., 72:5966-5975; Licciulli, et al. (2013) J. Biol. Chem., 288:29105-29114). However, development of this series of compounds was halted due to evidence of on-target toxicity related to cardiovascular events (Rudolph, et al. (2016) J. Med. Chem., 59:5520-5541). Similar findings were described in Pak2 knockout mice, using a tamoxifen regulated CAGG-Cre-ERT gene to delete the floxed Pak2 gene in adult mice (Radu, et al. (2015) Mol. Cell Biol., 35:3990-4005). In contrast, deletion of the closely related genes for Pak1 and Pak3 was not found to be required for viability, development, longevity, or fertility (Allen, et al. (2009) Blood 113:2695-2705; Hofmann, et al. (2004) J. Cell Sci., 117:4343-4354; Kelly, et al. (2012) Cell Logist., 2: 84-88). These combined findings indicate that PAK2 function is required in adult mice and that small molecules that inhibit PAK2 can be toxic or even lethal in humans.

Selective blockade of PAK1 is clinically useful in an animal model of neurofibromatosis type 2 (NF2). It has also been reported that deletion of the Pak1 gene, but not the Pak2 gene, was effective in slowing hearing loss and schwannoma growth in these mice, and that treatment with NVS-PAK1-1 showed a similar trend without obvious systemic toxicity (Hawley, et al. (2021) Human Mol. Genet., 30(17):1607-1617). As several cancer cells are PAK1-dependent, these findings indicate a path forward for PAK1-selective inhibitors.

Given that the signaling activity of PAK1 is mediated both by enzymatic and scaffolding functions (Sells, et al. (1997) Trends Cell Biol., 7:162-167; Sells, et al. (1997) Curr. Biol., 7:202-210), a degrader derived from NVS-PAK1-1 was synthesized to be more potent than the parental molecule, while simultaneously retaining its selectivity for PAK1 over PAK2. Herein, a collection of degraders (e.g., PROTACs) derived from conjugating NVS-PAK1-1 to a degron such as pomalidomide were synthesized and characterized. Degrons such as pomalidomide, thalidomide, and lenalidomide facilitate recruitment of the CRL4^{CRBN} ubiquitin ligase for substrate ubiquitination and eventual proteosome-mediated degradation. This degrader preserves the unique isoform-specific PAK1 inhibitor activity while simultaneously being capable of inducing PAK1 protein degradation.

Here, a PAK1-selective degrader (BJG-05-039) comprising the allosteric PAK1 inhibitor NVS-PAK1-1 (which has modest potency) conjugated to pomalidomide, a recruiter of the E3 ubiquitin ligase substrate adaptor Cereblon (CRBN), is provided. It is shown herein that BJG-05-039 induces degradation of PAK1, but not PAK2, and displays enhanced anti-proliferative effects relative to its parent compound in PAK1-dependent, but not PAK2-dependent, cell lines. These effects were further enhanced when drug efflux was reduced by a chemical inhibitor. BJG-05-039 is also compared to the parental inhibitor, a negative degrader (analog disabled for binding to CRBN), and to shRNA-mediated gene knockdown. Notably, BJG-05-039 promotes sustained PAK1 degradation and inhibition of downstream signaling effects at ten-fold lower dosage than NVS-PAK1-1. These findings indicate that selective PAK1 degradation confers more potent pharmacological effects compared with catalytic inhibition and highlight the advantages of PAK1-targeted degradation.

In accordance with the instant invention, PAK1 degraders are provided. In some embodiments, the PAK1 degrader effects the degradation of PAK1. In some embodiments, the PAK1 degrader is a proteolysis-targeting chimeric molecule (PROTAC). Generally, the PAK1 degrader is a molecule comprising a targeting ligand linked to a degron via a linker. Degrons bind to ubiquitin ligase, particularly an E3 ubiquitin ligase such as cereblon. The targeting ligand is capable of selectively binding to PAK1. In some embodiments, the targeting ligand selectively binds PAK1 compared to other PAKs, particularly PAK2. In some embodiments, the targeting ligand is at least 10 times, at least 20 times, at least 30 times, at least 40 times, at least 50 times, or more selective for PAK1 compared to other PAKs, particularly PAK2 (e.g., as determined by in vitro enzyme activity assay). In some embodiments, the PAK1 degrader selectively binds and/or degrades PAK1 compared to other PAKs, particularly PAK2. In some embodiments, the PAK1 degrader is at least 10 times, at least 20 times, at least 30 times, at least 40 times, at least 50 times, or more selective for PAK1 compared to other PAKs, particularly PAK2 (e.g., as determined by in vitro enzyme activity assay).

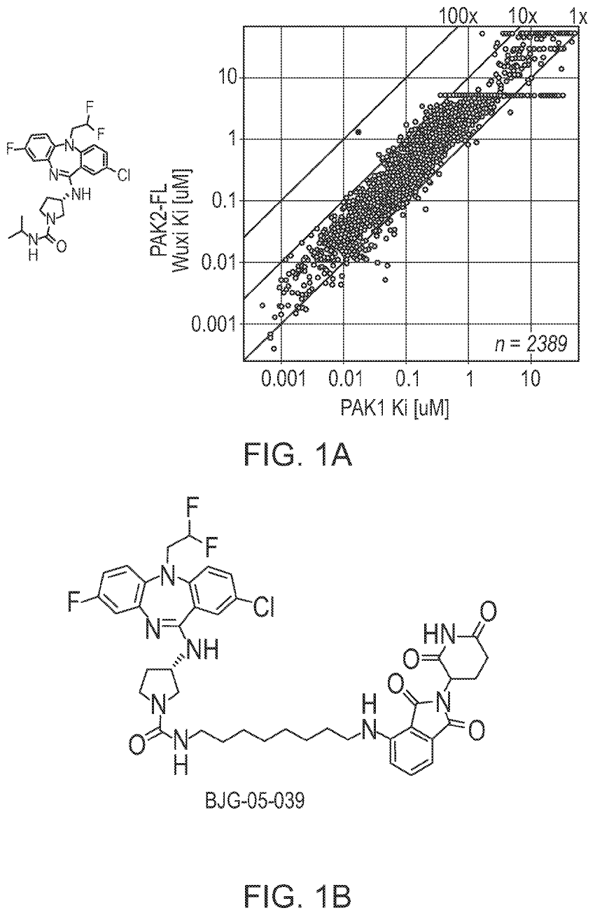

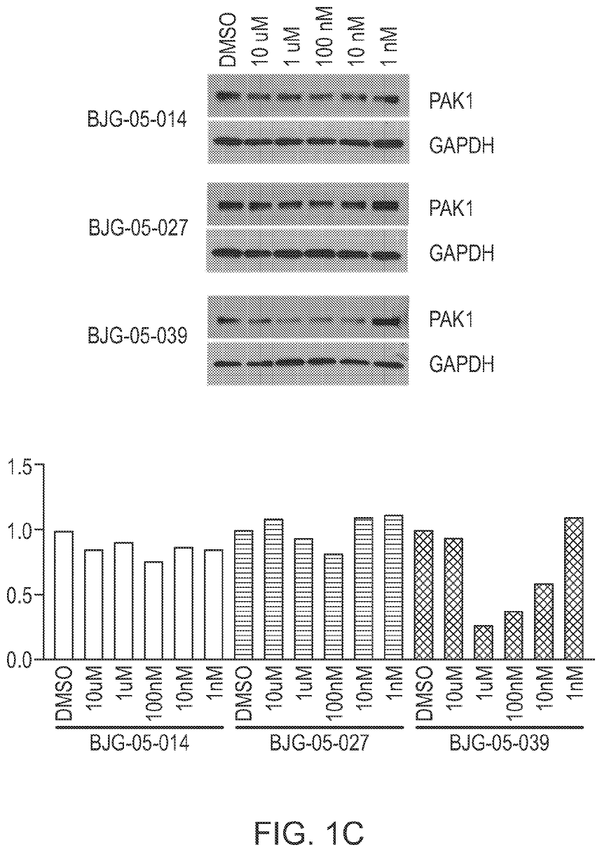

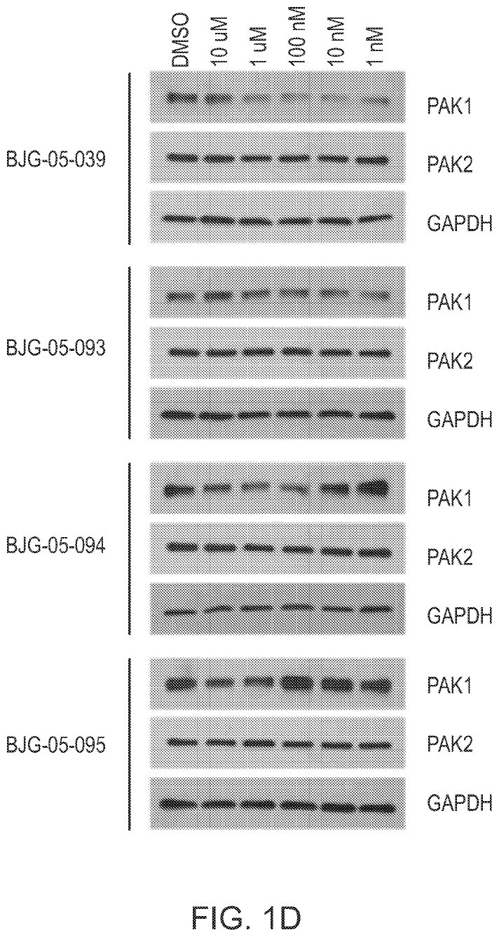

In some embodiments, the PAK1 degrader comprises NVS-PAK1-1 linked to a degron via a linker. The chemical structure of NVS-PAK1-1 is depicted in FIG. 1A. In some embodiments, the degron is linked to NVS-PAK-1 at the isopropyl urea. In some embodiments, the degron is linked to NVS-PAK-1 at the carbon after removal of —NH(isopropyl) from the isopropyl urea. In some embodiments, the degrader is linked to NVS-PAK-1 at the nitrogen after removal of the isopropyl from the isopropyl urea.

Broadly, the compounds of the invention are represented by Formula I or III:

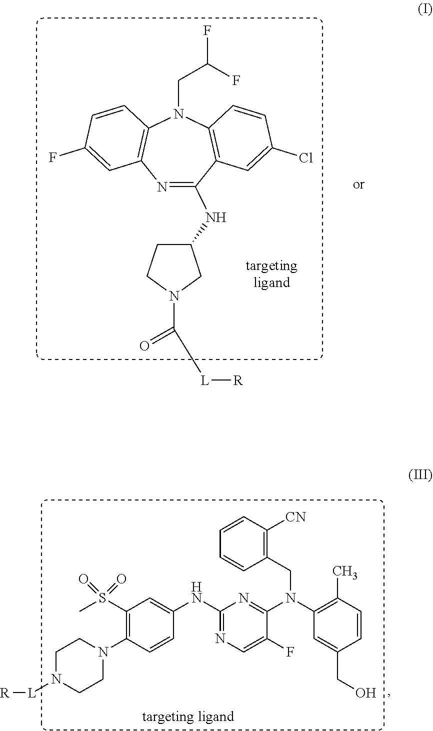

wherein:
wherein L is a linker and R is a degron,
the linker is a chemical moiety that covalently attaches the carbonyl carbon or the nitrogen to the degron; and
the degron is a ligand for an E3 ubiquitin ligase,
or a pharmaceutically acceptable salt or stereoisomer thereof.

### Linkers

As used herein, a linker (“L”) is a chemical moiety comprising one or more atoms that covalently attaches at least two compounds. The linkers of the instant invention link the targeting ligand with the degron. The linker can be linked to any synthetically feasible position of the compounds, but preferably in such a manner as to avoid blocking the compound's desired activity. Linkers are generally known in the art. In some embodiments, the linker may comprise 1 to about 100 atoms, 1 to about 50 atoms, 1 to about 40 atoms, 1 to about 30 atoms, 1 to about 25 atoms, 1 to about 20 atoms, 1 to about 15 atoms, or from 1 to about 10 atoms.

In some embodiments, the linker comprises a carbon chain, optionally substituted with one, two, three, or more optionally substituted heteroatoms (e.g., N, O, or S). In some embodiments, the linker is an optionally substituted alkyl or alkenyl. In some embodiments, the linker is a hydrocarbon.

In some embodiments, the linker is an optionally substituted hydrocarbon (e.g., an unbranched hydrocarbon), alkyl, or alkenyl chain comprising 15 or fewer carbons, 14 or fewer carbons, 13 or fewer carbons, 12 or fewer carbons, 11 or fewer carbons, 10 or fewer carbons, 9 or fewer carbons, or 8 or fewer carbons. In some embodiments, the linker is an optionally substituted hydrocarbon (e.g., unbranched hydrocarbon), alkyl, or alkenyl comprising at least 5 carbons, at least 6 carbons, at least 7 carbons, at least 8 carbons, at least 9 carbons, at least 10 carbons, at least 11 carbons, at least 12 carbons, at least 13 carbons, at least 14 carbons, or at least 15 carbons. In some embodiments, the linker is an optionally substituted hydrocarbon (e.g., unbranched hydrocarbon), alkyl, or alkenyl comprising 1 to 15 carbons, 1 to 12 carbons, 3 to 12 carbons, 3 to 10 carbons, 5 to 10 carbons, 6 to 10 carbons, 5 to 9 carbons, 5 to 8 carbons, 7 to 9 carbons, 8 to 10 carbons, or about 8 carbons. Typically, a hydrocarbon, alkyl, or alkenyl group, when substituted, may have 1, 2, 3, or more substituents. In some embodiments, the hydrocarbon, alkyl, or alkenyl is substituted by at least one oxo, C₁-C₃ alkyl (e.g., methyl), C₂-C₄ alkenyl, C₁-C₃ alkoxy (e.g., methoxy), C₁-C₃ monoalkylamino (—NH(alkyl)), C₁-C₃ dialkylamino (—N(alkyl)₂), halogen, —OH, —SH, —NH₂, —COOH, —CN, and/or —NO₂. In some embodiments, the alkyl or alkenyl linker is a heteroalkyl or heteroalkenyl linker, wherein at least one (e.g., from 1 to about 4) carbon is replaced with a heteroatom (e.g., sulfur, oxygen, or nitrogen). In some embodiments, the linker is a bond or an alkylene chain (e.g., having 1-20 alkylene units) which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker is of formula L0:

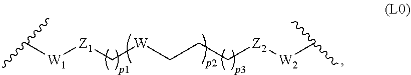

or stereoisomer thereof, wherein
p1 is an integer selected from 0 to 6;
p2 is an integer selected from 0 to 12;
p3 is an integer selected from 0 to 15;
each W is independently absent, CH₂, O, S, NR₁₀, or C(O)NR₁₀;
each R₁₀ is independently hydrogen or C₁-C₆ alkyl;
W₁ and W₂ are independently absent, (CH₂)₁₋₃, O, or NH; and
Z₁ and Z₂ are independently absent, —O—, —S—, —N(R₁₀)—, —C≡C—, —C(O)—, —C(O)O—, —OC(O)—, —OC(O)O—, —C(NOR₁₀)—, —C(O)N(R₁₀)—, —C(O)N(R₁₀)C(O)—, —C(O)N(R₁₀)C(O)N(R₁₀)—, —N(R₁₀)C(O)—, —N(R₁₀)C(O)N(R₁₀)—, —N(R₁₀)C(O)O—, —OC(O)N(R₁₀)—, —C(NR₁₀)—, —N(R₁₀)C(NR₁₀)—, —C(NR₁₀)N(R₁₀)—, —N(R₁₀)C(NR₁₀)N(R₁₀)—, —OB(Me)O—, —S(O)₂—, —OS(O)—, —S(O)O—, —S(O)—, —OS(O)₂—, —S(O)₂O—, —N(R₁₀)S(O)₂—, —S(O)₂N(R₁₀)—, —N(R₁₀)S(O)—, —S(O)N(R₁₀)—, —N(R₁₀)S(O)₂N(R₁₀)—, —N(R₁₀)S(O)N(R₁₀)—, C₃-C₁₂ carbocyclene, 3- to 12-membered heterocyclene, or 5- to 12-membered heteroarylene;
   wherein the linker is covalently bonded to a degron via the

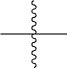

next to W₂, or the linker is covalently bonded to a degron via the

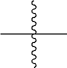

next to W₁.

In some embodiments, formula L0 is of formula L0a-L0h:

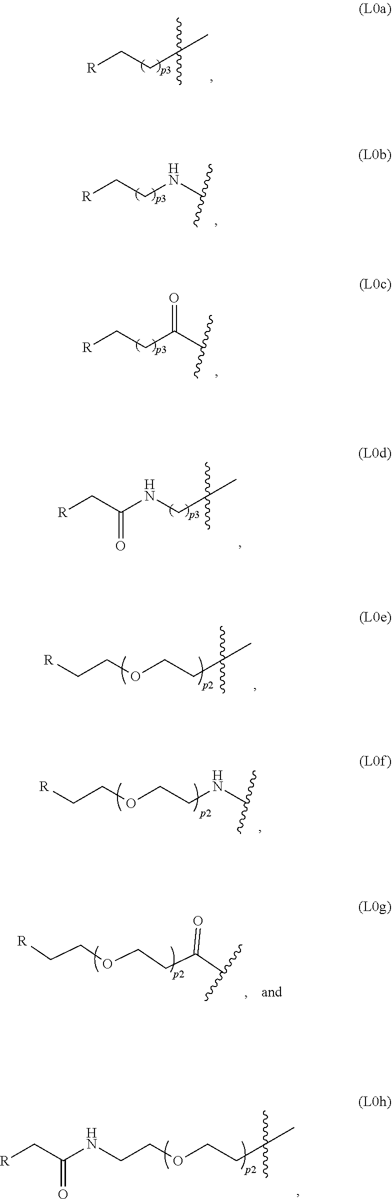

wherein R represents the degron.

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

In some embodiments, the linker includes an alkylene chain having 1-15 alkylene units that is interrupted by and/or terminating in NH, C(O), or NHC(O). In some embodiments, the linker includes an alkylene chain having 1-10 alkylene units that is interrupted by and/or terminating in NH, C(O), or NHC(O). In some embodiments, the linker includes an alkylene chain having 1-6 alkylene units that is interrupted by and/or terminating in NH, C(O), or NHC(O). In some embodiments, the linker includes an alkylene chain having 1-15 alkylene units. In some embodiments, the linker includes an alkylene chain having 1-10 alkylene units. In some embodiments, the linker includes an alkylene chain having 1-6 alkylene units.

Representative examples of alkylene linkers that may be suitable for use in the compounds of the present invention include the following:

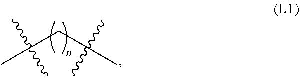

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

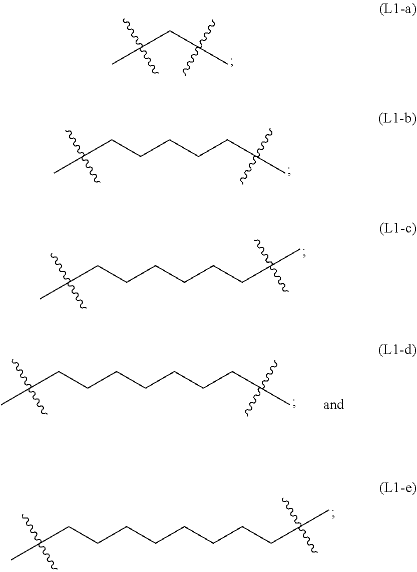

alkylene chains terminating in various functional groups (as described above), examples of which are as follows:

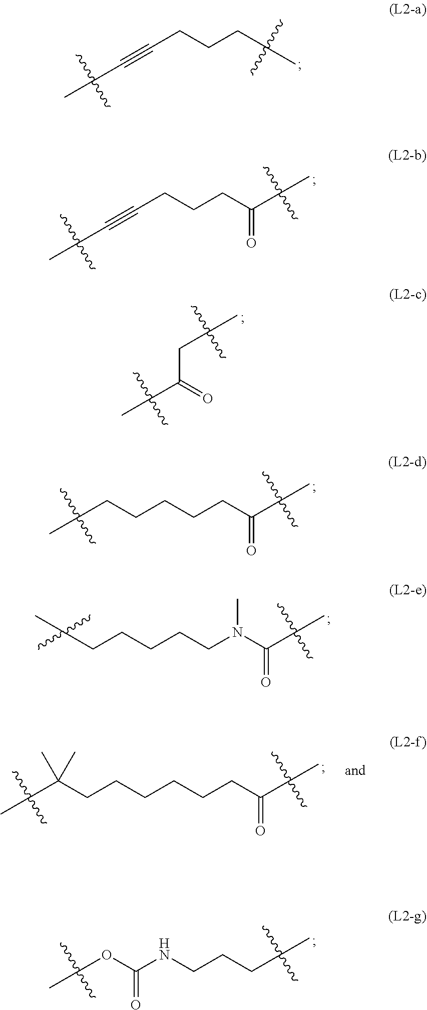

alkylene chains interrupted with various functional groups (as described above), examples of which are as follows:

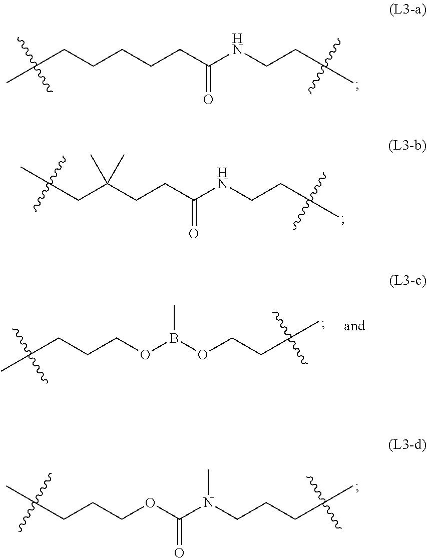 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with a heterocyclene group, e.g.,

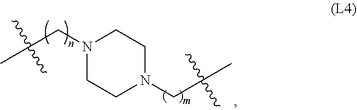

wherein m and n are independently integers of 0-10, examples of which include:

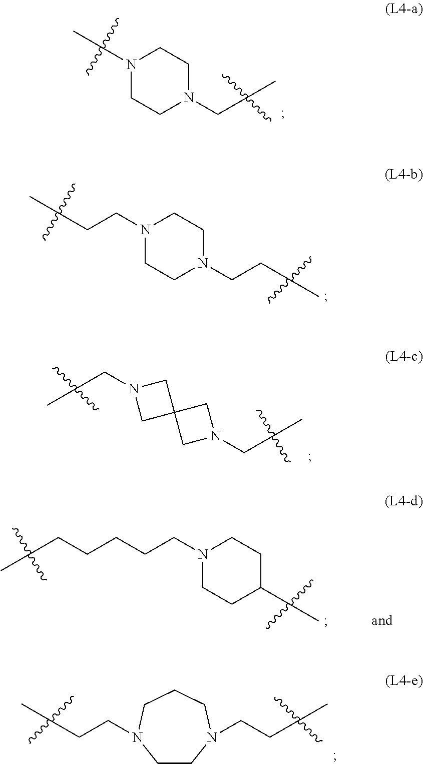 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by an amide, a heterocyclene and/or an aryl group, examples of which include:

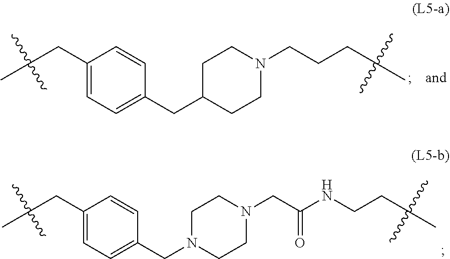 [SMILES: *CCCN1CCC(Cc2ccc(C*)cc2)CC1 ; *CCNC(=O)CN1CCN(Cc2ccc(C*)cc2)CC1]

alkylene chains interrupted by a heterocyclene, an aryl group, and a heteroatom, examples of which include:

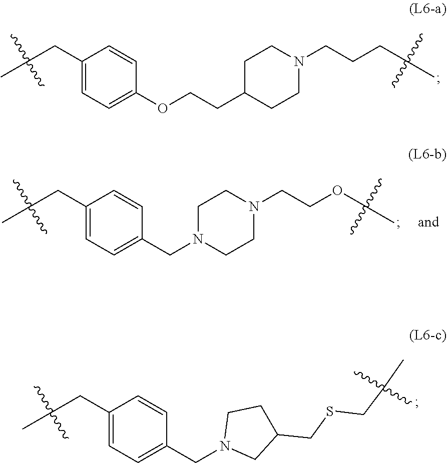 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by a heteroatom such as N, O or B, e.g.,

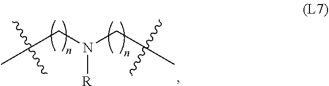

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

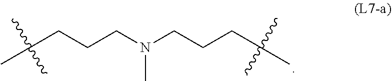 [SMILES: *CCCN(C)CCC*]

In some embodiments, the linker comprises polyethylene glycol (PEG). In some embodiments, the linker comprises the formula —(O—CH₂—CH₂)ₙ—, wherein n=2 to 6, 2 to 5, 2 to 4, or 2 to 3. In some embodiments, the linker is a polyethylene glycol (PEG) chain which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker includes a polyethylene glycol chain having 1-5 PEG units and terminates in NH, C(O), or NHC(O). In some embodiments, the linker includes a polyethylene glycol chain having 1-5 PEG units.

Representative examples of linkers that include a polyethylene glycol chain include:

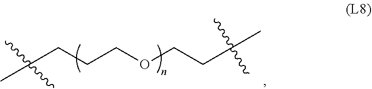

wherein n is an integer of 2-10, examples of which include:

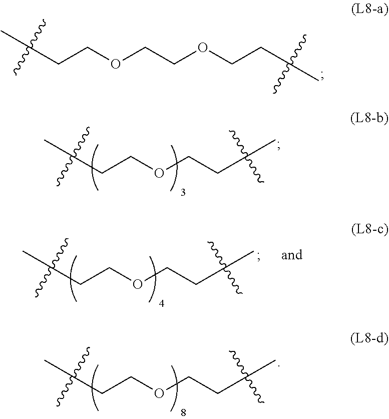

In some embodiments, the polyethylene glycol linker may terminate in a functional group, examples of which are as follows:

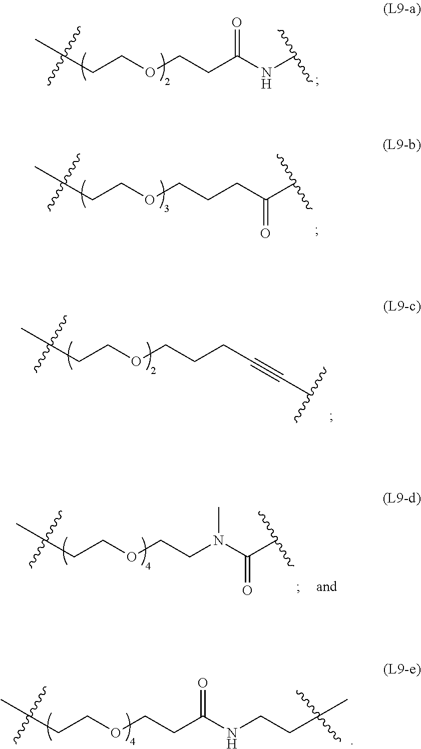

In some embodiments, the linker is a linker depicted in Table 1 or Table 2. In some embodiments, the linker is represented by any one of structures:

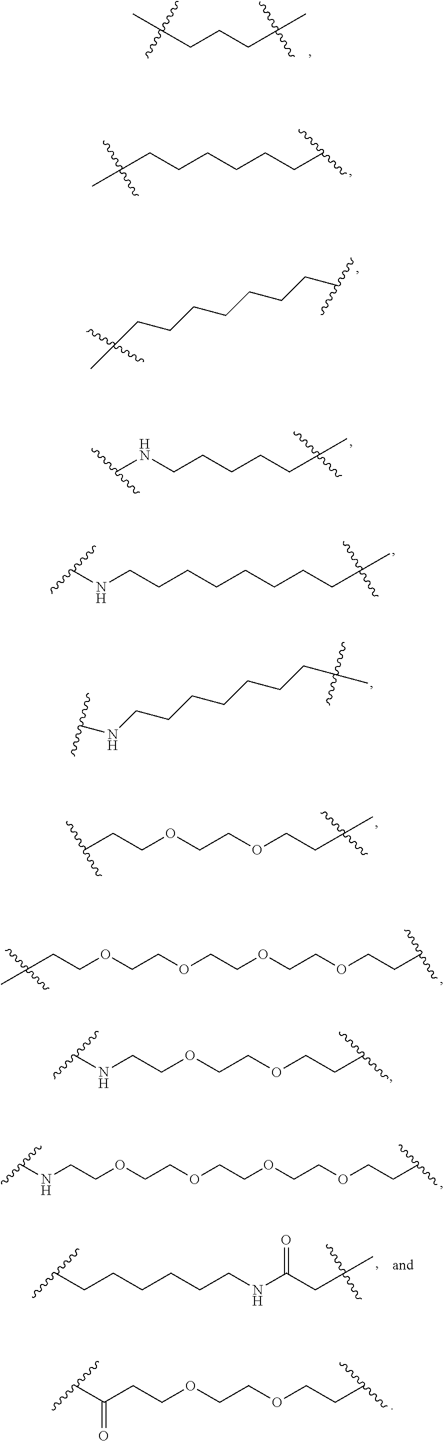

Therefore, in some embodiments, compounds of the present invention may be represented by any one of the following structures:

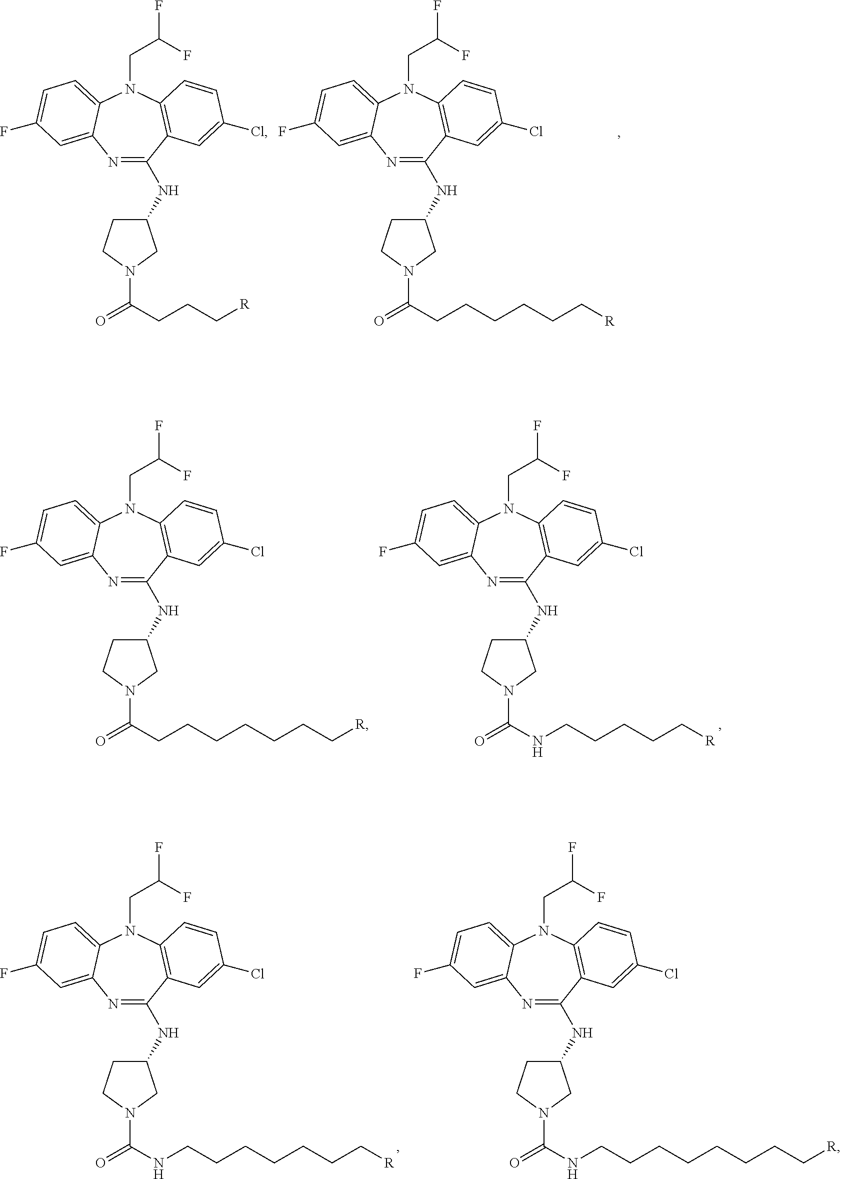

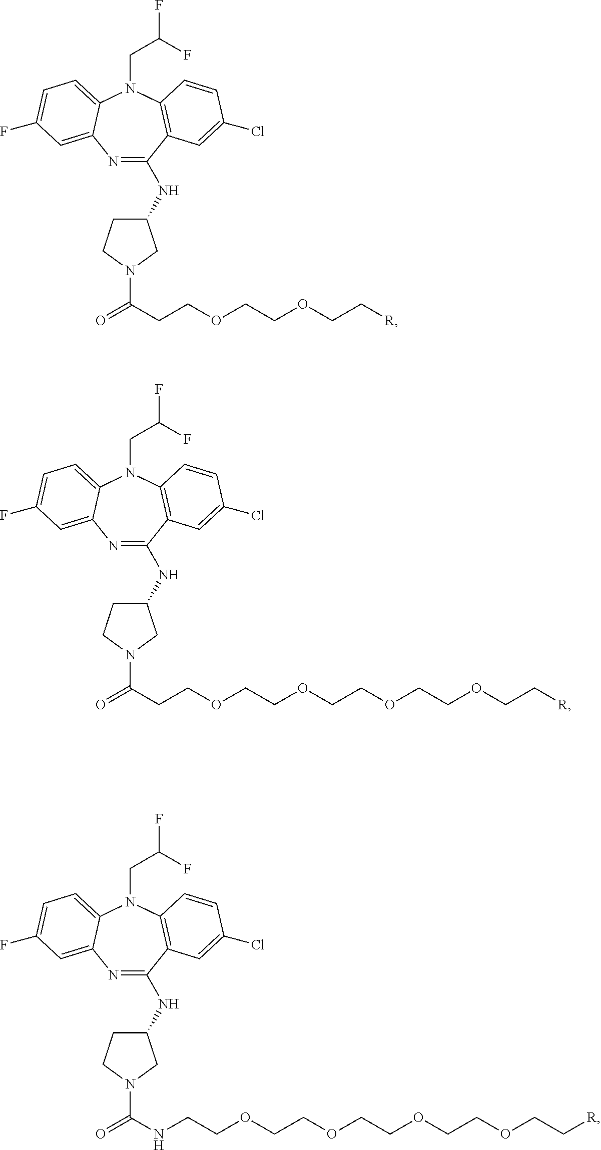

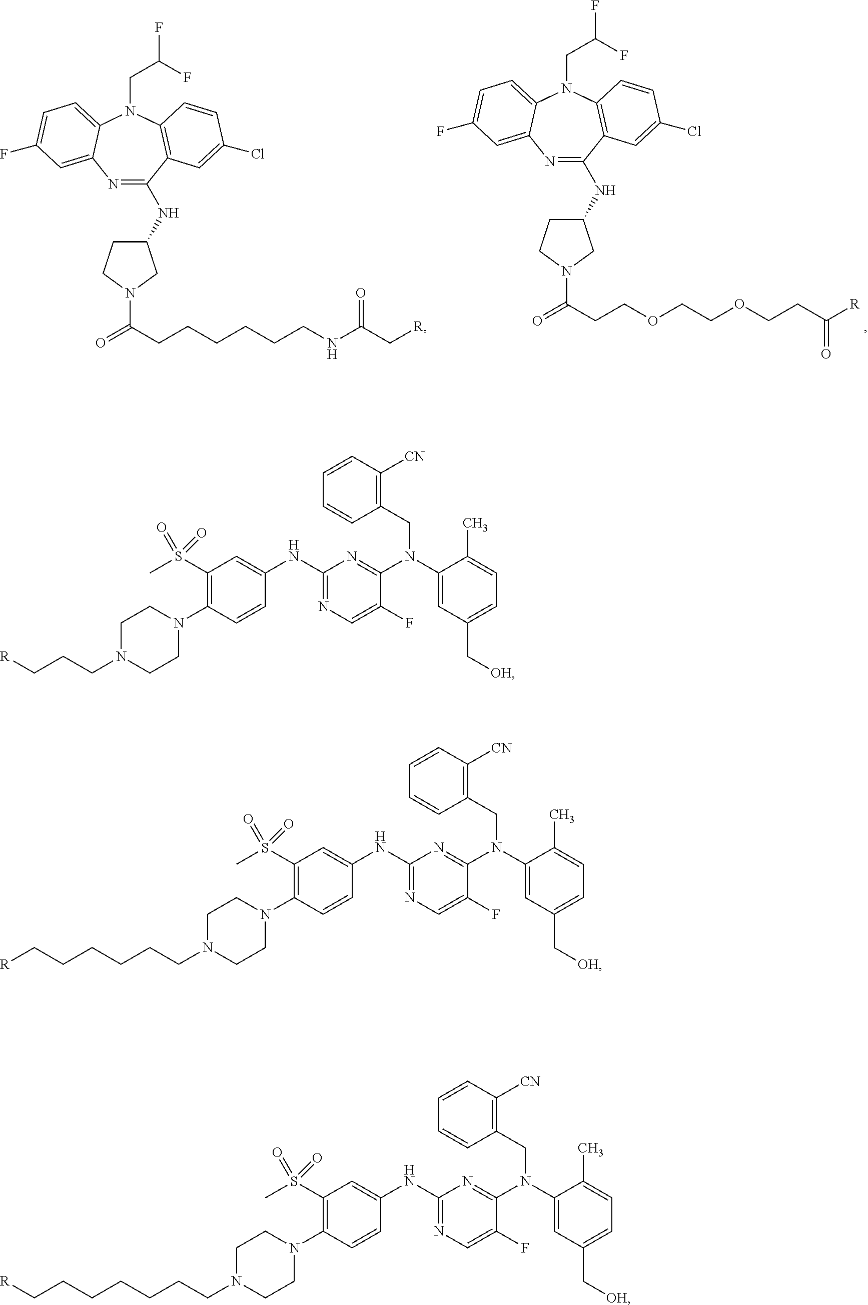

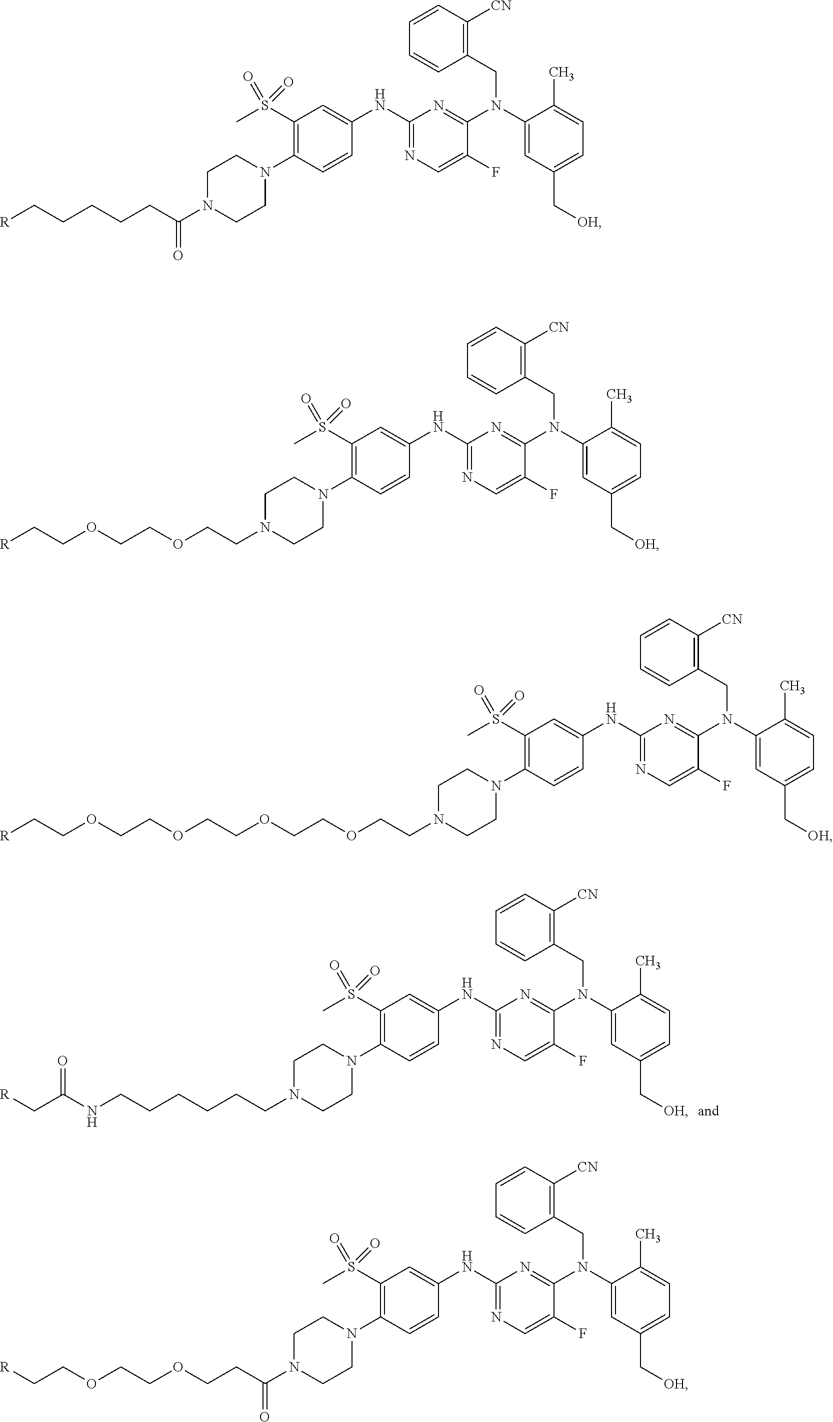

or a pharmaceutically acceptable salt or stereoisomer thereof, wherein R represents the degron. Any of the degrons disclosed herein e.g., in paragraphs 0073-0086 and 0088-0089, below, may be used.

In some embodiments, the compound of Formula I is represented by Formula II:

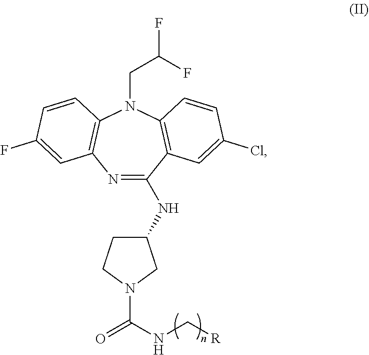

wherein R is a degron and n=1 to 15, or a pharmaceutically acceptable salt or stereoisomer thereof. In some embodiments, n=1 to 12, 3 to 12, 3 to 10, 5 to 10, 6 to 10, 5 to 9, 5 to 8, 7 to 9, 8 to 10, or about 8. In some embodiments, n is at least 4 or at least 5. In some embodiments, n is 10 or fewer, 9 or fewer, or 8 or fewer.

In some embodiments, the compound of Formula T is represented by Formula II′:

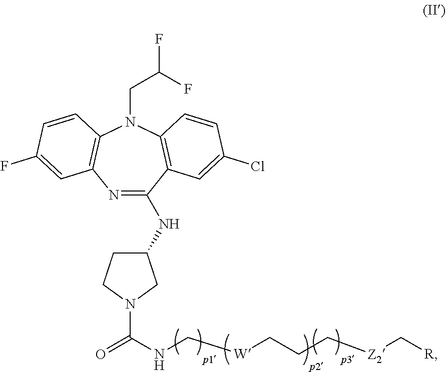

or a pharmaceutically acceptable salt or stereoisomer thereof, wherein
p1′ is an integer selected from 0 to 6;
p2′ is an integer selected from 0 to 6;
p3′ is an integer selected from 0 to 8;
each W′ is independently absent, CH₂, O, S, NR₁₀, or C(O)NR₁₀;
each R₁₀ is independently hydrogen or C₁-C₆ alkyl;
Z₂′ is absent, —O—, —S—, —N(R₁₀)—, —C≡C—, —C(O)—, —C(O)O—, —OC(O)—, —OC(O)O—, —C(NOR₁₀)—, —C(O)N(R₁₀)—, —C(O)N(R₁₀)C(O)—, —C(O)N(R₁₀)C(O)N(R₁₀)—, —N(R₁₀)C(O)—, —N(R₁₀)C(O)N(R₁₀)—, —N(R₁₀)C(O)O—, —OC(O)N(R₁₀)—, —C(NR₁₀)—, —N(R₁₀)C(NR₁₀)—, —C(NR₁₀)N(R₁₀)—, —N(R₁₀)C(NR₁₀)N(R₁₀)—, —OB(Me)O—, —S(O)₂—, —OS(O)—, —S(O)O—, —S(O)—, —OS(O)₂—, —S(O)₂O—, —N(R₁₀)S(O)₂—, —S(O)₂N(R₁₀)—, —N(R₁₀)S(O)—, —S(O)N(R₁₀)—, —N(R₁₀)S(O)₂N(R₁₀)—, —N(R₁₀)S(O)N(R₁₀)—, C₃-C₁₂ carbocyclene, 3- to 12-membered heterocyclene, or 5- to 12-membered heteroarylene; and
R is a degron.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the degron is a compound (e.g., a targeting moiety or ligand) that binds to ubiquitin ligase, particularly an E3 ubiquitin ligase. Without being bound by any particular theory, the degron recruits ubiquitin ligase, particularly the E3 ubiquitin ligase, to tag/label PAK1 for ubiquitination and degradation through the proteasome. Examples of E3 ubiquitin ligases include, without limitation: Von Hippel-Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase. In some embodiments, the E3 ubiquitin ligase is the E3 ubiquitin ligase substrate adaptor cereblon (CRBN).

The degron that binds the E3 ubiquitin ligase may be derived from or comprise an E3 ubiquitin ligase ligand. Examples of degrons (and methods of synthesizing them) are known in the art (see, e.g., Bricelj et al. (2021) Front. Chem., 9:707317; incorporated herein by reference for E3 ubiquitin ligase ligands and methods of synthesis). Examples of degrons include, without limitation: pomalidomide, 4-hydroxythalidomide, alkyl-connected thalidomide derivatives, lenalidomide, thalidomide, VHL ligand 1 (VHL-1), VHL ligand 2 (VHL-2), iberdomide, thalidomide-propargyl, eragidomide, cereblon modulator 1 (CAS 1860875-51-9), lenalidomide hemihydrate, thalidomide fluoride, thalidomide-OH, lenalidomide-Br, thalidomide D4, IAP ligand LCL-161, MDM2 ligand Nutlin-3a, and MDM2 ligand idasanutlinde. Examples of degrons are also provided in U.S. Patent Application Publication No. 2022/0047709 (e.g., structures D1-a, D1-b, D1-c; incorporated herein by reference).

In some embodiments, representative examples of degrons that bind cereblon are represented by D1:

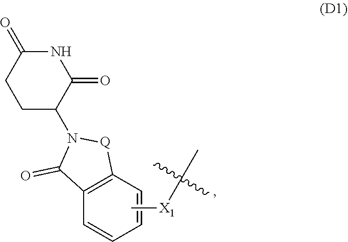

wherein Q is CH₂ or C(O); and
X₁ is a bond, CH₂, O, NH, or C≡C.

In some embodiments, Q is CH₂.

In some embodiments, Q is C(O).

In some embodiments, X₁ is O.

In some embodiments, X₁ is NH.

In some embodiments, X₁ is CH₂.

In some embodiments, X₁ is C≡C.

In some embodiments, X₁ is a bond.

In some embodiments, Q is CH₂ and X₁ is O. In some embodiments, Q is CH₂ and X₁ is NH. In some embodiments, Q is CH₂ and X₁ is CH₂. In some embodiments, Q is CH₂ and X₁ is C≡C. In some embodiments, Q is CH₂ and X₁ is a bond.

In some embodiments, Q is C(O) and X₁ is O. In some embodiments, Q is C(O) and X₁ is NH. In some embodiments, Q is C(O) and X₁ is CH₂. In some embodiments, Q is C(O) and X₁ is C≡C. In some embodiments, Q is C(O) and X₁ is a bond.

In some embodiments, the degron is of Formula D1a-D1t.

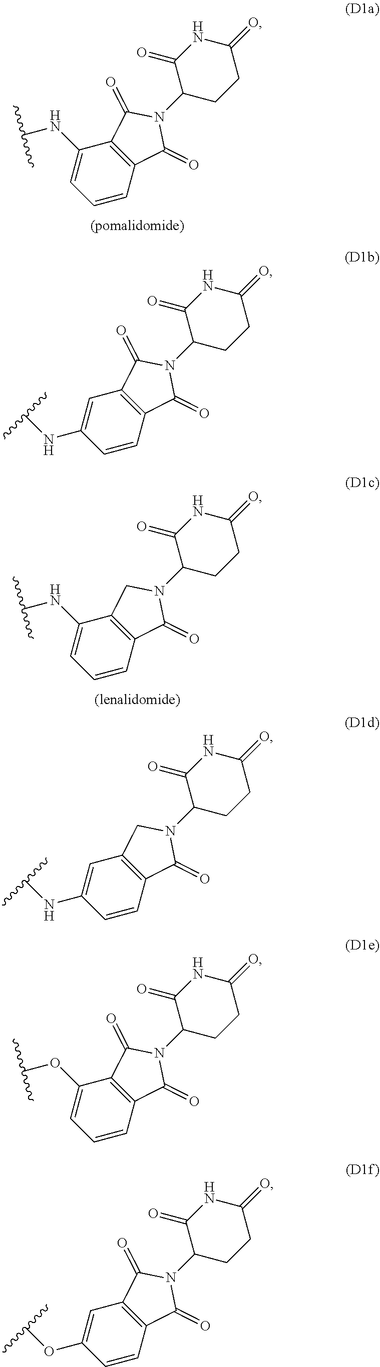 [SMILES: *Nc1cccc2c1C(=O)N(C1CCC(=O)NC1=O)C2=O ; *Nc1ccc2c(c1)C(=O)N(C1CCC(=O)NC1=O)C2=O ; *Nc1cccc2c1CN(C1CCC(=O)NC1=O)C2=O ; *Nc1ccc2c(c1)CN(C1CCC(=O)NC1=O)C2=O ; *Oc1cccc2c1C(=O)N(C1CCC(=O)NC1=O)C2=O ; *Oc1ccc2c(c1)C(=O)N(C1CCC(=O)NC1=O)C2=O]

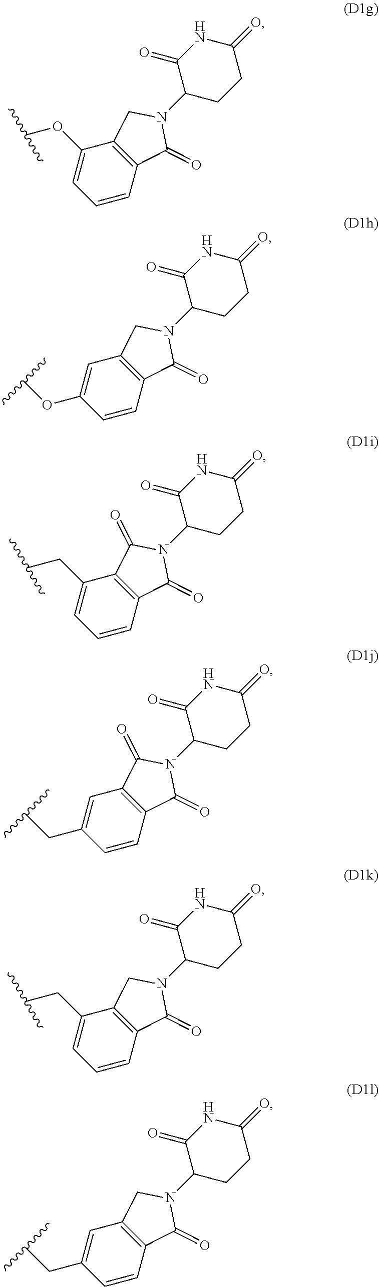 [SMILES: *Oc1cccc2c1CN(C1CCC(=O)NC1=O)C2=O ; *Oc1ccc2c(c1)CN(C1CCC(=O)NC1=O)C2=O ; *Cc1cccc2c1C(=O)N(C1CCC(=O)NC1=O)C2=O ; *Cc1ccc2c(c1)C(=O)N(C1CCC(=O)NC1=O)C2=O ; *Cc1cccc2c1CN(C1CCC(=O)NC1=O)C2=O ; *Cc1ccc2c(c1)CN(C1CCC(=O)NC1=O)C2=O]

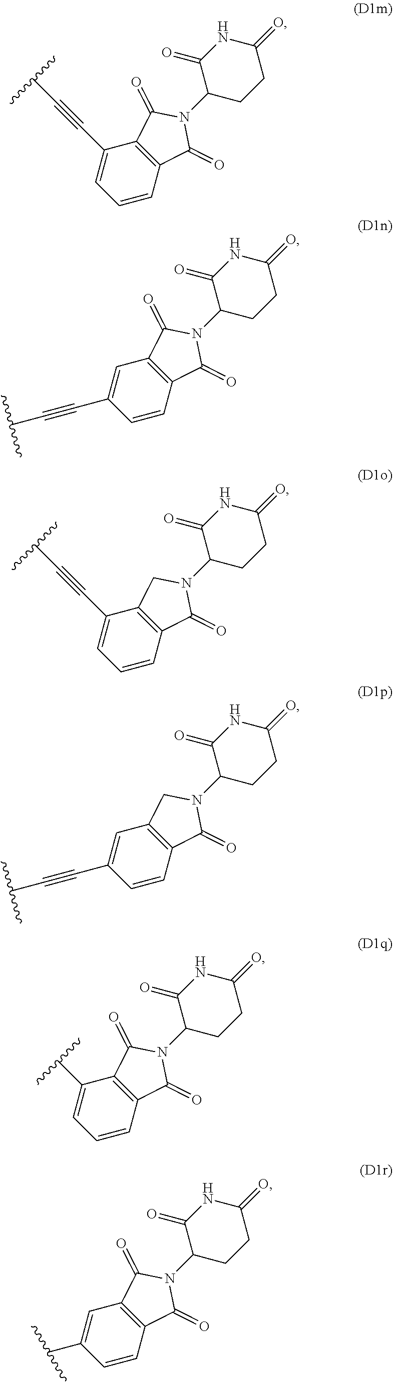 [SMILES: *C#Cc1cccc2c1C(=O)N(C1CCC(=O)NC1=O)C2=O ; *C#Cc1ccc2c(c1)C(=O)N(C1CCC(=O)NC1=O)C2=O ; *C#Cc1cccc2c1CN(C1CCC(=O)NC1=O)C2=O ; *C#Cc1ccc2c(c1)CN(C1CCC(=O)NC1=O)C2=O ; *c1cccc2c1C(=O)N(C1CCC(=O)NC1=O)C2=O ; *c1ccc2c(c1)C(=O)N(C1CCC(=O)NC1=O)C2=O]

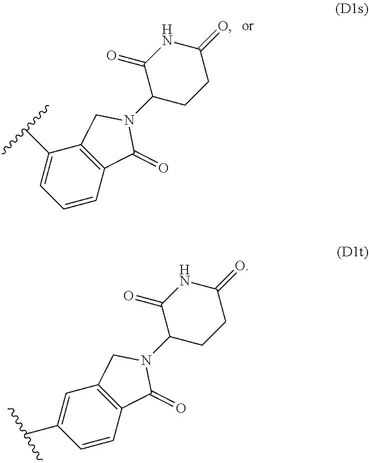 [SMILES: *c1cccc2c1CN(C1CCC(=O)NC1=O)C2=O ; *c1ccc2c(c1)CN(C1CCC(=O)NC1=O)C2=O]

In some embodiments, the degron is thalidomide or an analog thereof. In some embodiments, the degron is lenalidomide or an analog thereof. In some embodiments, the degron is pomalidomide or an analog thereof.

In some embodiments, the compounds of the present invention may be represented by any of the following structures:

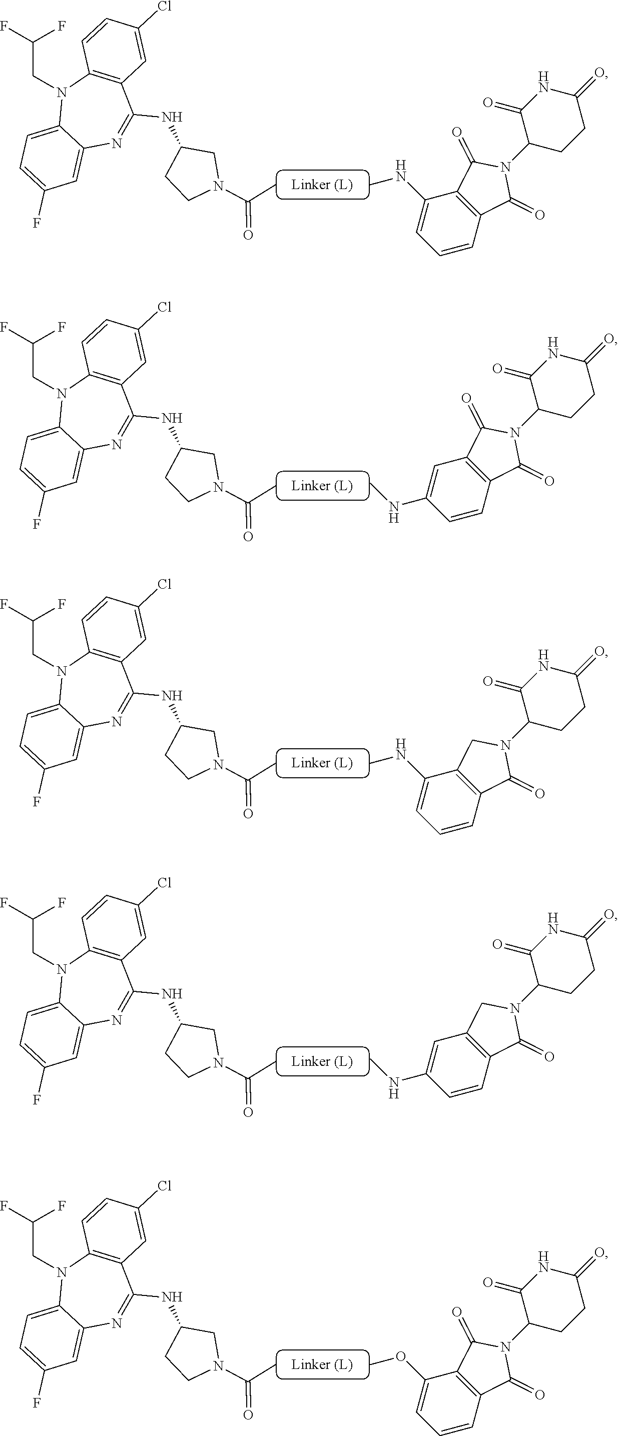

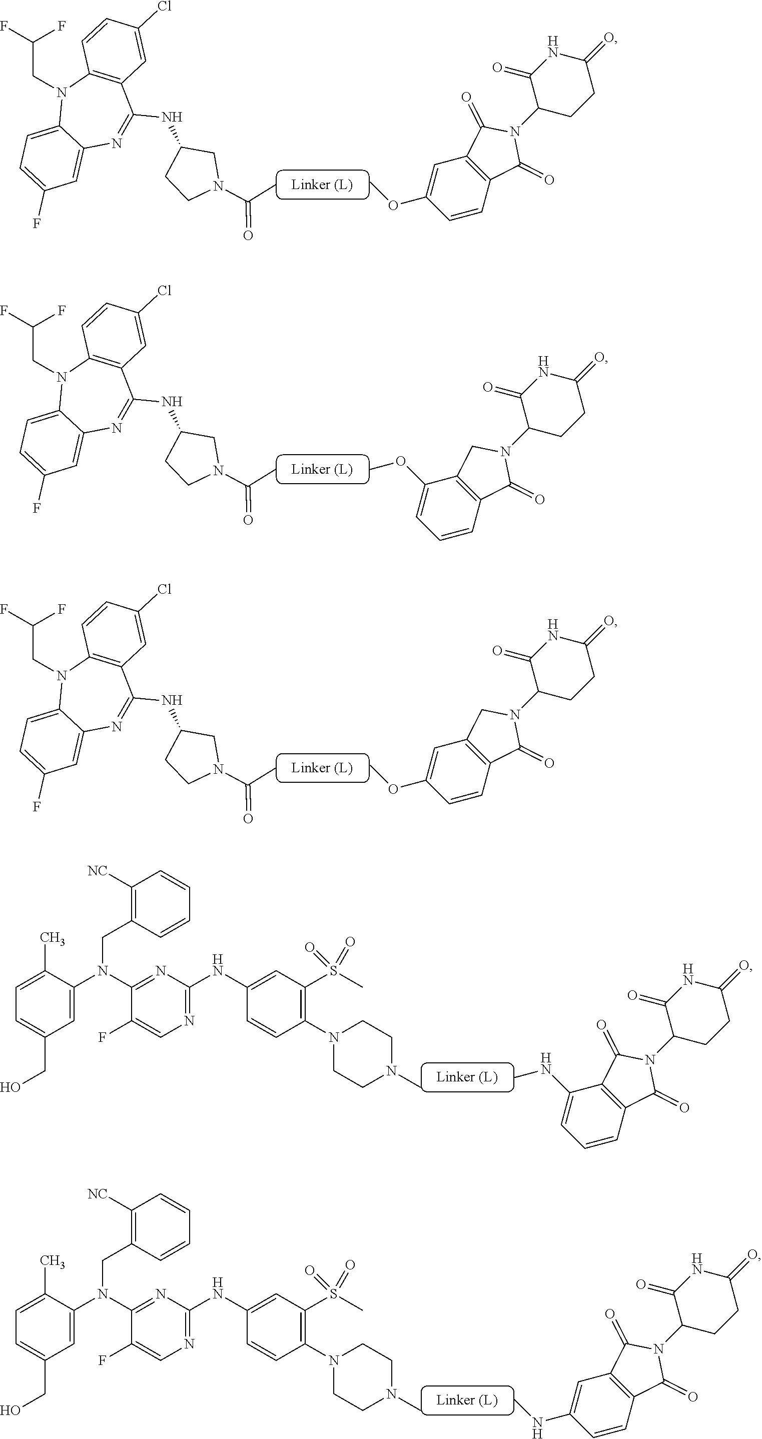

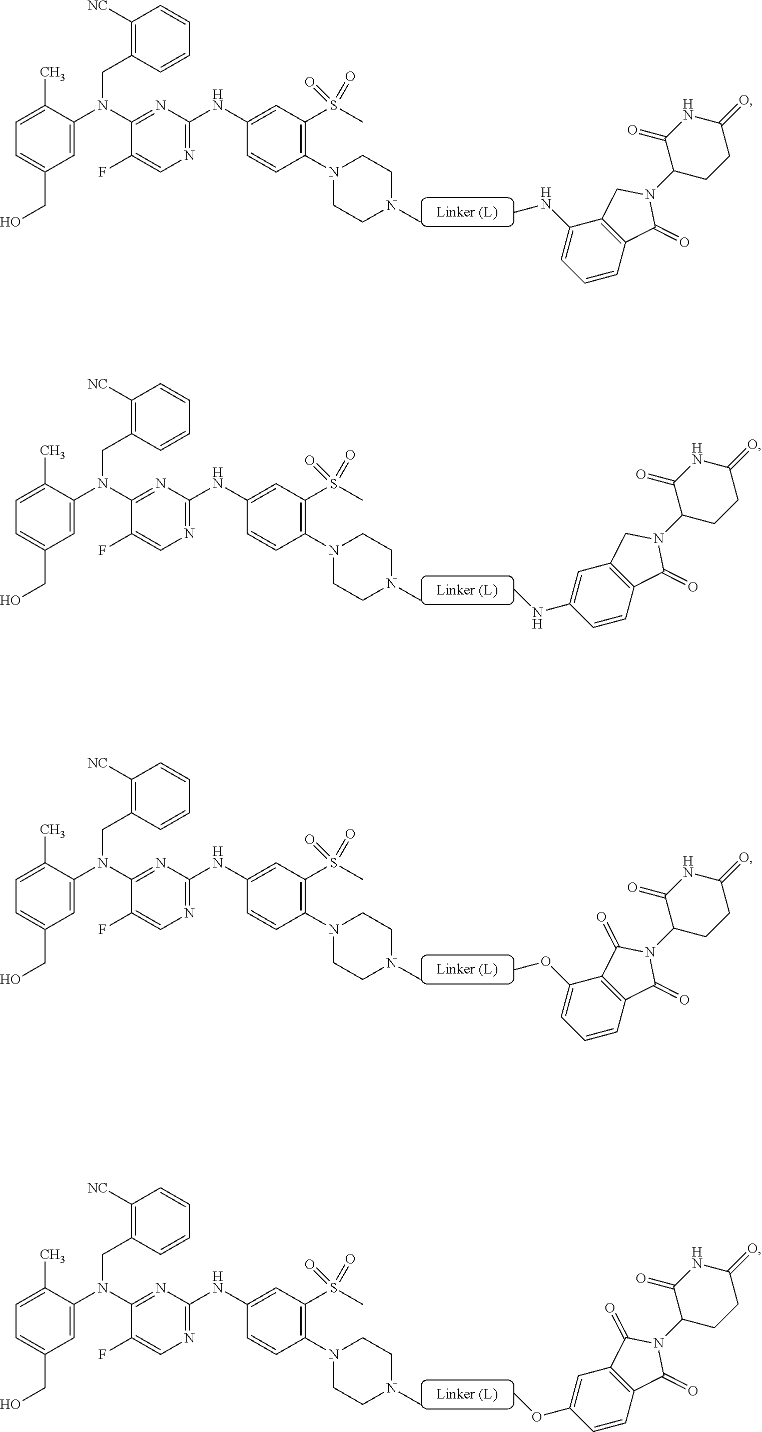

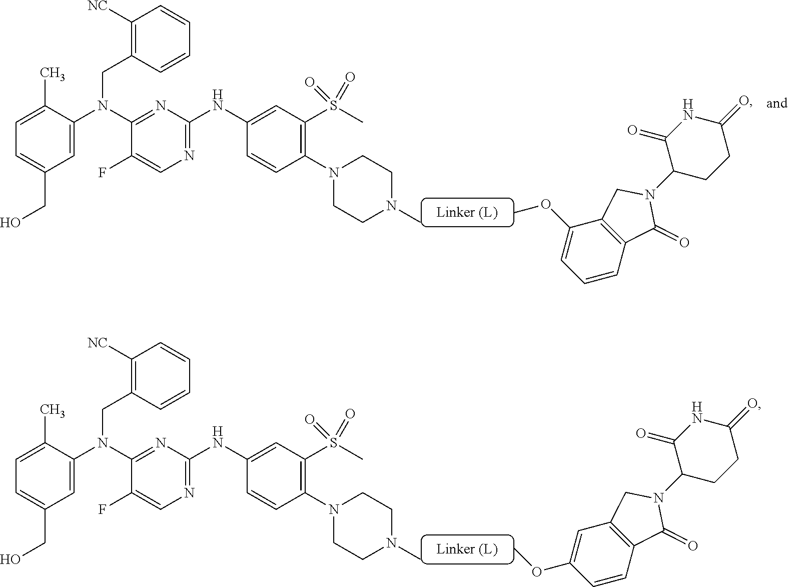 [SMILES: Cc1ccc(CO)cc1N(Cc1ccccc1C#N)c1nc(Nc2ccc(N3CCN(C)CC3)c(S(C)(=O)=O)c2)ncc1F ; COc1cccc2c1CN(C1CCC(=O)NC1=O)C2=O ; Cc1ccc(CO)cc1N(Cc1ccccc1C#N)c1nc(Nc2ccc(N3CCN(C)CC3)c(S(C)(=O)=O)c2)ncc1F ; COc1ccc2c(c1)CN(C1CCC(=O)NC1=O)C2=O]

or a pharmaceutically acceptable salt or stereoisomer thereof. Any of the linkers disclosed in paragraphs 0055-0068, above, may be used.

Representative examples of degrons that bind VHL are represented by any one of structures (D2-a) to (D2-f):

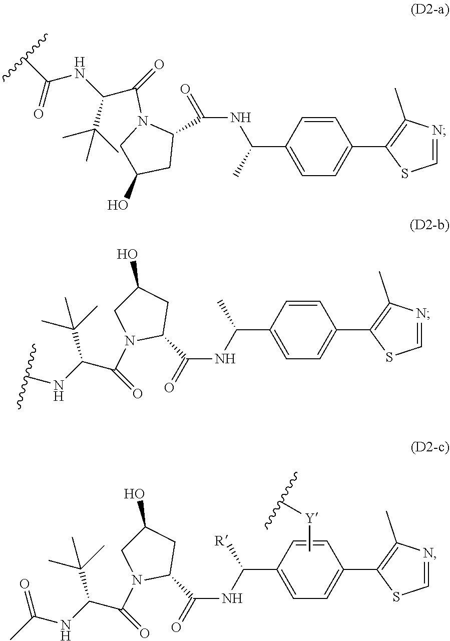

wherein Y′ is a bond, NH, O or CH₂ and R′ is H or methyl;

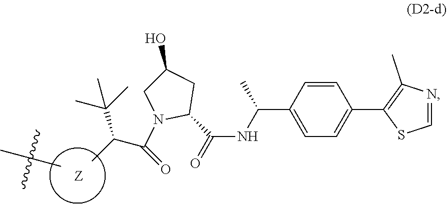

wherein Z is a C₅-C₆ carbocycle or a C₅-C₆ heterocyclic group;

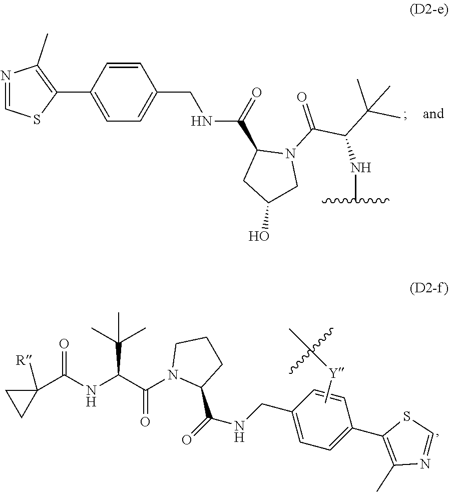

wherein Y″ is a bond, NH, O or CH₂ and R″ is F or CN,
or a stereoisomer thereof.

In some embodiments, Z is

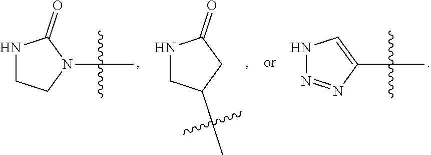 [SMILES: *N1CCNC1=O ; *C1CNC(=O)C1 ; *c1c[nH]nn1]

In some embodiments, the compounds of the present invention may be represented by an of the following structures:

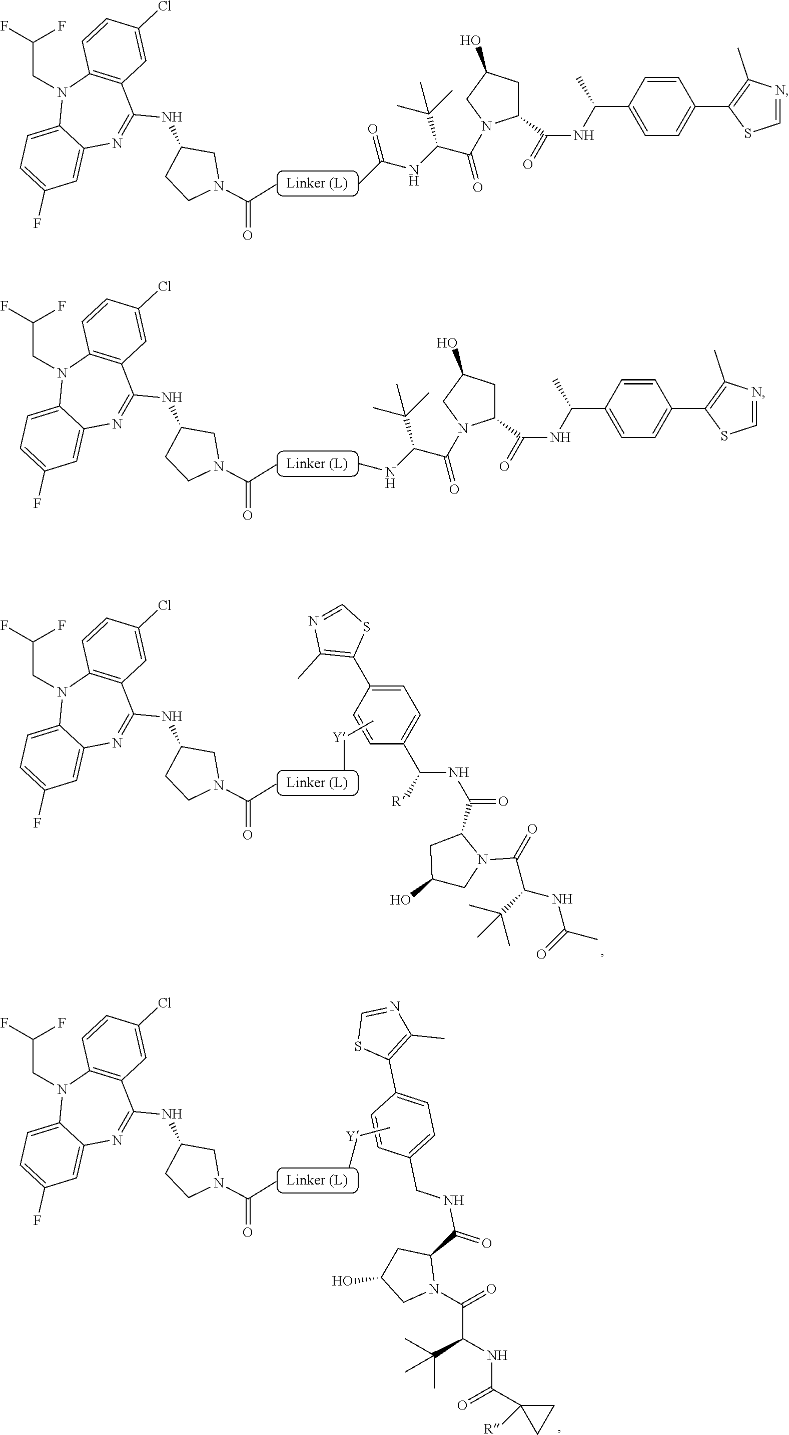

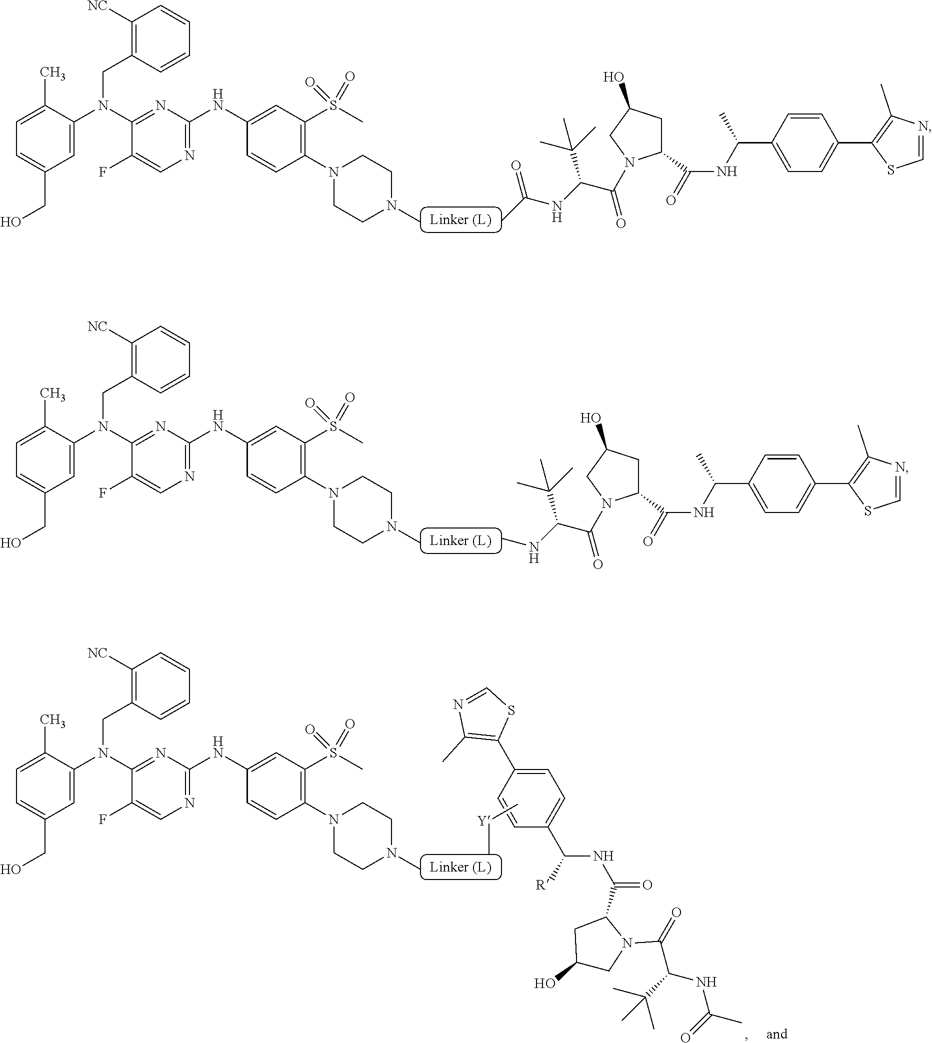

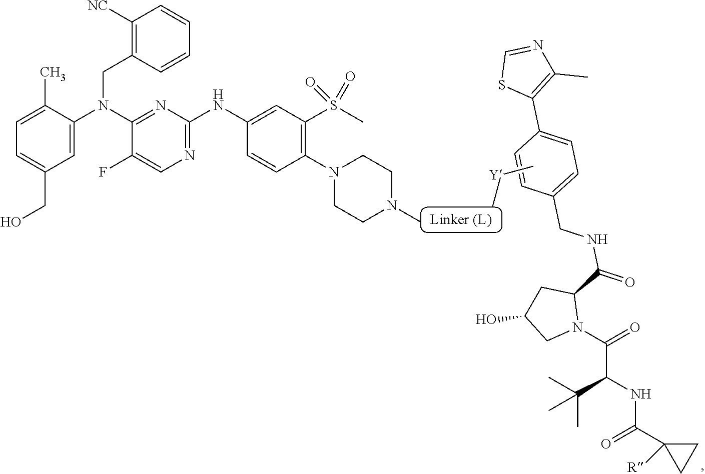

or a pharmaceutically acceptable salt or stereoisomer thereof. Any of the linkers disclosed in paragraphs 0055-0068, above, may be used.

In some embodiments, compounds of the present invention are represented by any one of the following structures:

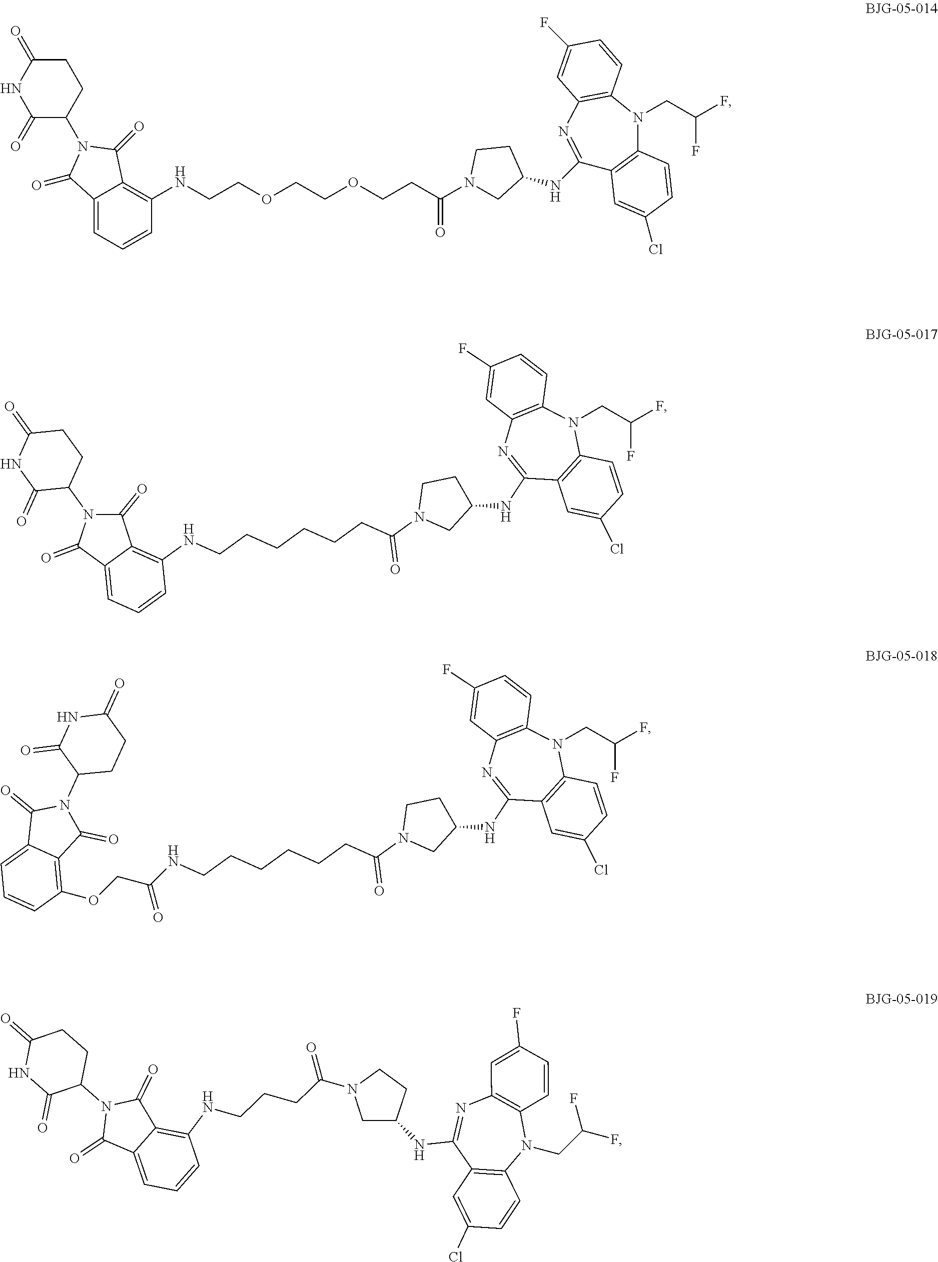 [SMILES: O=C1CCC(N2C(=O)c3cccc(NCCOCCOCCC(=O)N4CC[C@H](NC5=Nc6cc(F)ccc6N(CC(F)F)c6ccc(Cl)cc65)C4)c3C2=O)C(=O)N1 ; O=C1CCC(N2C(=O)c3cccc(NCCCCCCC(=O)N4CC[C@H](NC5=Nc6cc(F)ccc6N(CC(F)F)c6ccc(Cl)cc65)C4)c3C2=O)C(=O)N1 ; O=C(COc1cccc2c1C(=O)N(C1CCC(=O)NC1=O)C2=O)NCCCCCCC(=O)N1CC[C@H](NC2=Nc3cc(F)ccc3N(CC(F)F)c3ccc(Cl)cc32)C1 ; O=C1CCC(N2C(=O)c3cccc(NCCCC(=O)N4CC[C@H](NC5=Nc6cc(F)ccc6N(CC(F)F)c6ccc(Cl)cc65)C4)c3C2=O)C(=O)N1]

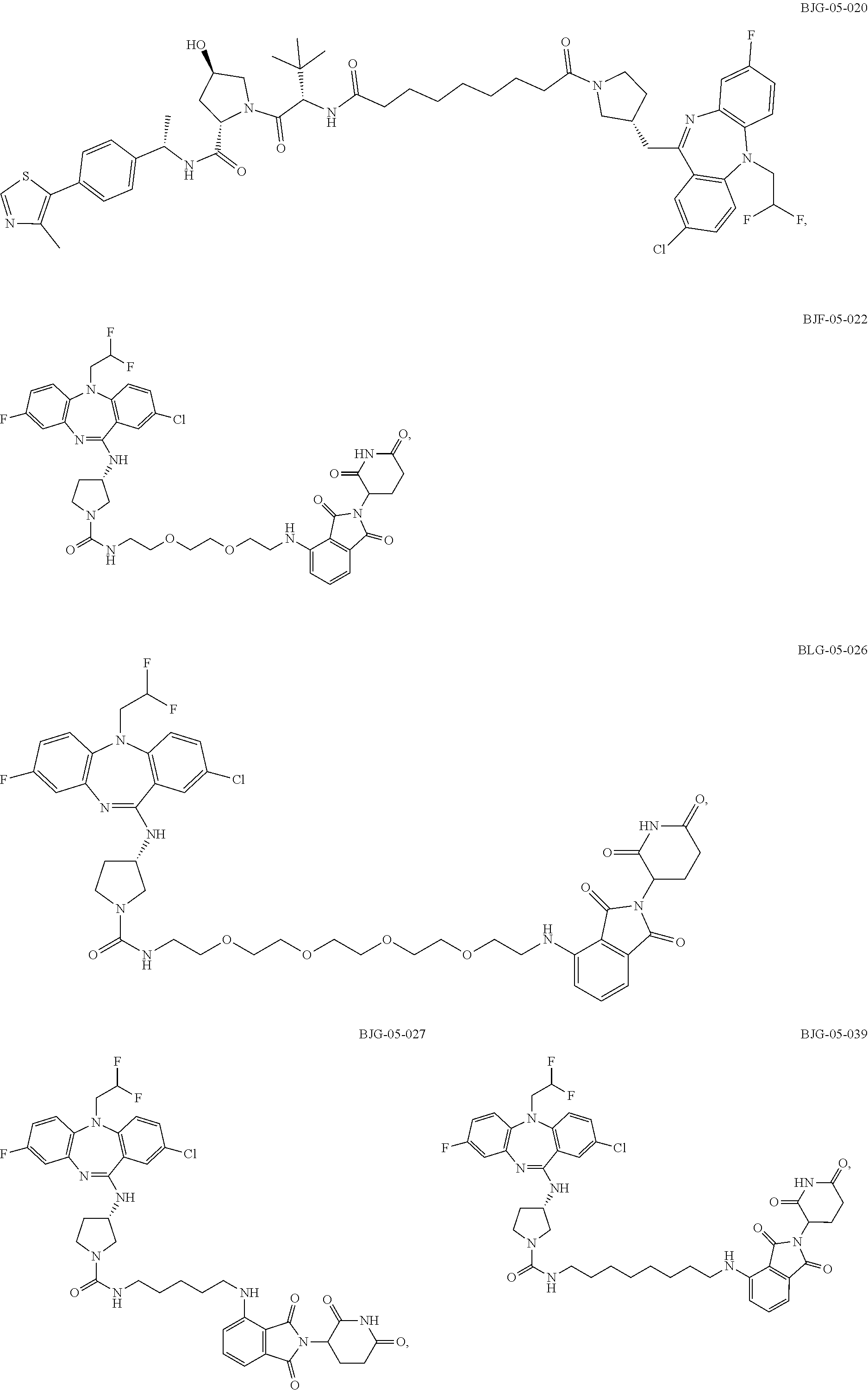

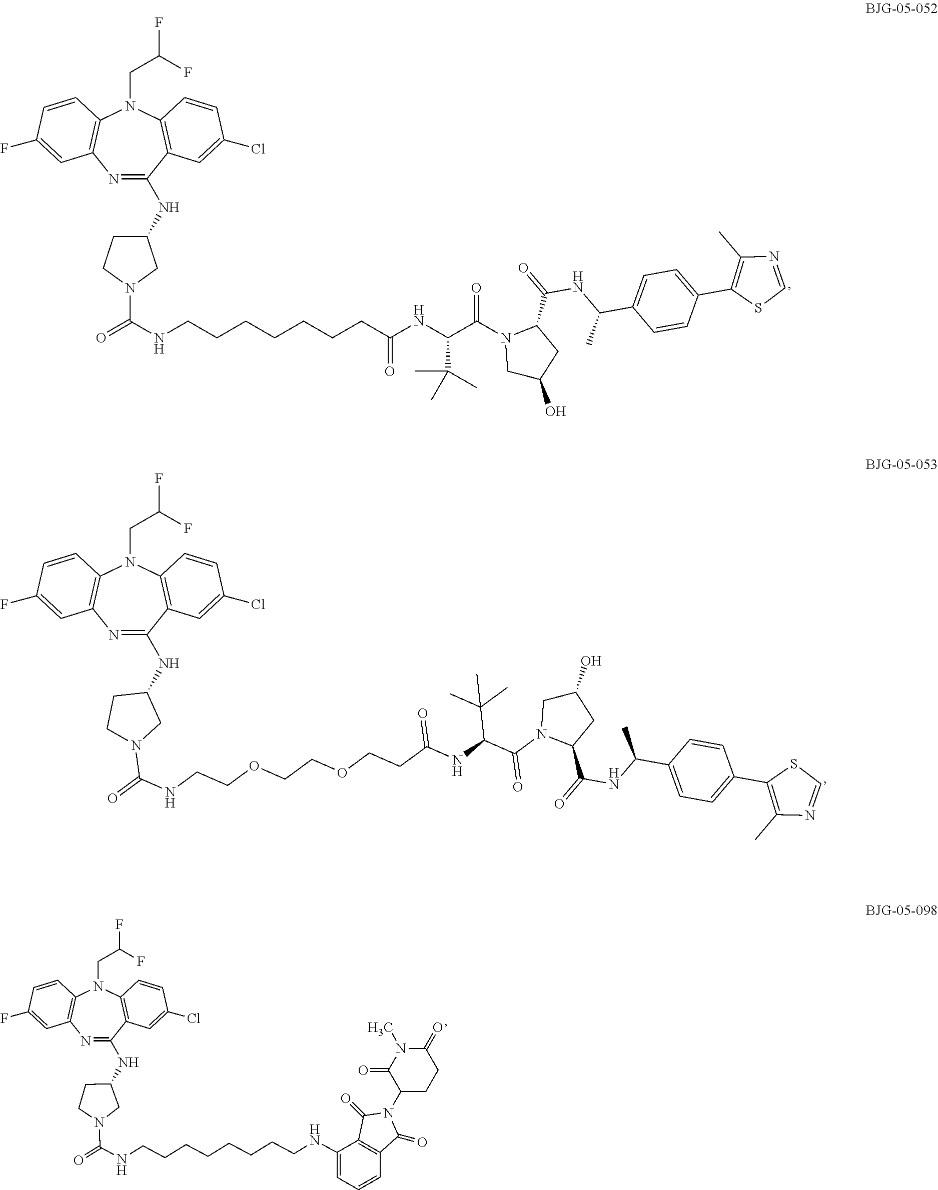 [SMILES: Cc1ncsc1-c1ccc([C@H](C)NC(=O)[C@@H]2C[C@@H](O)CN2C(=O)[C@@H](NC(=O)CCCCCCCNC(=O)N2CC[C@H](NC3=Nc4cc(F)ccc4N(CC(F)F)c4ccc(Cl)cc43)C2)C(C)(C)C)cc1 ; Cc1ncsc1-c1ccc([C@H](C)NC(=O)[C@@H]2C[C@@H](O)CN2C(=O)[C@@H](NC(=O)CCOCCOCCNC(=O)N2CC[C@H](NC3=Nc4cc(F)ccc4N(CC(F)F)c4ccc(Cl)cc43)C2)C(C)(C)C)cc1 ; CN1C(=O)CCC(N2C(=O)c3cccc(NCCCCCCCCNC(=O)N4CC[C@H](NC5=Nc6cc(F)ccc6N(CC(F)F)c6ccc(Cl)cc65)C4)c3C2=O)C1=O]

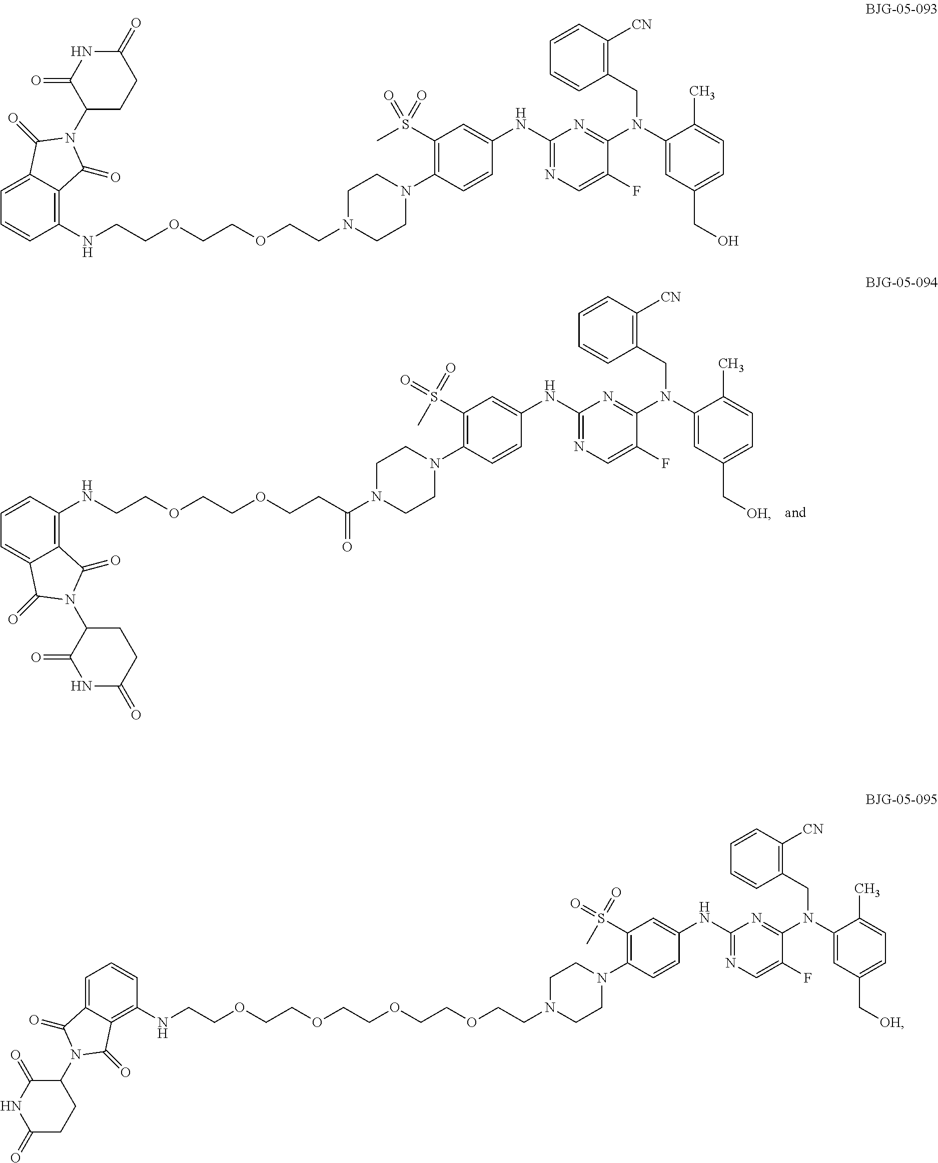 [SMILES: Cc1ccc(CO)cc1N(Cc1ccccc1C#N)c1nc(Nc2ccc(N3CCN(CCOCCOCCNc4cccc5c4C(=O)N(C4CCC(=O)NC4=O)C5=O)CC3)c(S(C)(=O)=O)c2)ncc1F ; Cc1ccc(CO)cc1N(Cc1ccccc1C#N)c1nc(Nc2ccc(N3CCN(C(=O)CCOCCOCCNc4cccc5c4C(=O)N(C4CCC(=O)NC4=O)C5=O)CC3)c(S(C)(=O)=O)c2)ncc1F ; Cc1ccc(CO)cc1N(Cc1ccccc1C#N)c1nc(Nc2ccc(N3CCN(CCOCCOCCOCCOCCNc4cccc5c4C(=O)N(C4CCC(=O)NC4=O)C5=O)CC3)c(S(C)(=O)=O)c2)ncc1F]

or pharmaceutically acceptable salt or stereoisomer thereof.

Compounds of formula (I-III) may be in the form of a free acid or free base, or a pharmaceutically acceptable salt. As used herein, the term “pharmaceutically acceptable” indicates approval by a regulatory agency of the Federal or a state government or listed in the U.S. Pharmacopeia or other generally recognized pharmacopeia for use in animals, and more particularly in humans. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Compounds of formula (I-III) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or EZ, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the compound of formula (I-III) is an isotopic derivative in that it has at least one desired isotopic substitution of an atom, at an amount above the natural abundance of the isotope, i.e., enriched.

In addition, compounds of formula (I-III) embrace N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In some embodiments, the present invention is directed to a method for making a compound of formula (I-III) or a pharmaceutically acceptable salt or stereoisomer thereof. Broadly, the compounds or pharmaceutically acceptable salts or stereoisomers thereof, may be prepared by any process known to be applicable to the preparation of chemically related compounds. The compounds of the present invention will be better understood in connection with the synthetic schemes that described in various working examples that illustrate non-limiting methods by which the compounds of the invention may be prepared.

### Pharmaceutical Compositions

The compounds and compositions of the present invention can be administered by any suitable route, for example, by injection (e.g., for local (direct, including to or within a tumor) or systemic administration), or other modes of administration. In a particular embodiment, the compound is administered systemically. The composition may be administered by any suitable means, including intratumoral, parenteral, intramuscular, intravenous, orally, intraarterial, intraperitoneal, subcutaneous, intraarterial, intrarectal, and intramuscular administration. In a particular embodiment, the compound is administered intravenously, intramuscularly, or subcutaneously. In a particular embodiment, the compounds and compositions of the present invention are administered by direct injection (e.g., to the tumor and/or the surrounding area).

Except insofar as any conventional carrier is incompatible with the compound to be administered, its use in the pharmaceutical composition is contemplated. In a particular embodiment, the carrier is a pharmaceutically acceptable carrier. The instant invention also encompasses kits comprising a composition comprising a compound of the instant invention and at least one carrier (e.g., a pharmaceutically acceptable carrier) and/or a composition comprising an additional therapy and at least one carrier (e.g., a pharmaceutically acceptable carrier).

In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington: The Science and Practice of Pharmacy, 21st edition, Philadelphia, PA. Lippincott Williams & Wilkins. 2005. The pharmaceutical composition of the present invention can be prepared, for example, in liquid form, or can be in dried powder form (e.g., lyophilized for later reconstitution).

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. For example, the compounds may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the compounds in the chosen medium may be varied and the medium may be chosen based on the desired route of administration of the pharmaceutical preparation. Except insofar as any conventional media or agent is incompatible with the compounds to be administered, its use in the pharmaceutical preparation is contemplated.

Selection of a suitable pharmaceutical preparation depends upon the method of administration chosen. For example, the molecules of the invention may be administered by direct injection into any cancerous tissue or into the area surrounding the cancer. In this instance, a pharmaceutical preparation comprises the molecules dispersed in a medium that is compatible with the cancerous tissue.

As stated hereinabove, agents of the instant invention may also be administered parenterally by intravenous injection into the blood stream, or by subcutaneous, intramuscular, intratumor, intrathecal, or intraperitoneal injection. Pharmaceutical preparations for parenteral injection are known in the art. If parenteral injection is selected as a method for administering the molecules, steps should be taken to ensure that sufficient amounts of the molecules reach their target cells to exert a biological effect. The lipophilicity of the molecules, or the pharmaceutical preparation in which they are delivered, may have to be increased so that the molecules can arrive at their target locations. Methods for increasing the lipophilicity of a molecule are known in the art.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral, topical, or parenteral. In preparing the molecule in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment in combination with other standard chemotherapies. The dosage units of the molecules may be determined individually or in combination with each chemotherapy according to greater shrinkage and/or reduced growth rate of tumors.

### Dosage Amounts

As used herein, the term, “therapeutically effective amount” refers to an amount of a compound of formula (I-III), or a pharmaceutically acceptable salt or a stereoisomer thereof; or a composition including a compound of formula (I-III), or a pharmaceutically acceptable salt or a stereoisomer thereof, effective in producing the desired therapeutic response in a particular patient in need thereof. Therefore, the term “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated with a disease or disorder (e.g., cancer), or which simply kills or inhibits the growth of diseased (e.g., cancer) cells, or reduces the amount of PAK1, PAK2, NF1, and/or NF2 in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, for example, Goodman and Gilman's The Pharmacological Basis of Therapeutics, 10th Edition, A. Gilman, J. Hardman and L. Limbird, eds., McGraw-Hill Press, 155-173, 2001), which is incorporated herein by reference in its entirety.

Compounds of formula (I-III), and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of a compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders by reducing the level or activity of PAK1, PAK2, NF1, and/or NF2. The methods entail administration of a therapeutically effective amount of a compound formula (I-III), or a pharmaceutically acceptable salt or stereoisomer thereof, to a subject in need thereof.

The diseases or disorders are characterized or mediated by aberrant PAK1, PAK2, NF1, and/or NF2 activity (e.g., elevated levels of PAK1, PAK2, NF1, and/or NF2 or otherwise functionally abnormal PAK1, PAK2, NF1, and/or NF2, e.g., mutant PAK1, PAK2, NF1, and/or NF2 activity, relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

As explained herein, compounds of the instant invention are useful for the targeted treatment, inhibition, and/or prevention of diseases and disorders expressing PAK1. In some embodiments, the disease or disorder is characterized by elevated PAK1 expression and/or activity (e.g., compared to healthy or normal cells). In some embodiments, the disease or disorder is characterized by amplification of the PAK1 gene. In some embodiments, the compounds of the instant invention are effective for killing cancer cells and/or slowing or reducing tumor growth (e.g., a benign or malignant tumor). In some embodiments, the compounds of the instant invention are effective for treating, inhibiting, and/or preventing neurofibromatosis type 2 (NF2) and/or neurofibromatosis type 1 (NF1).

In accordance with the instant invention, compositions and methods for inhibiting (e.g., reducing or slowing), treating, and/or preventing cancer in a subject are provided. In a particular embodiment, the methods comprise administering to a subject in need thereof at least one compound of the instant invention. The compounds of the instant invention may be administered in a composition comprising the compound and at least one pharmaceutically acceptable carrier. In some embodiments, the compound is of Formula (I), (II), or (III) or the compound is BJG-05-039, or a pharmaceutically acceptable salt or stereoisomer thereof. The compounds of the present invention can be used to directly kill cancer cells or inhibit or slow cancer cell growth. The compounds may also be used to increase the sensitivity of cancer cells, making them more susceptible to other therapeutics (e.g., chemotherapeutics, radiotherapy, etc.).

The methods of the instant invention can be used to inhibit, prevent, and/or treat any cancer in a subject in need thereof, particularly a human. In a particular embodiment, the cancer is a solid tumor. The cancer may be chemo-resistant and/or radio-resistant. In some embodiments, the cancer overexpresses PAK1; has elevated PAK1 activity; and/or has an amplification of the PAK1 gene (e.g., compared to normal or healthy cells). Examples of cancer that can be treated by the methods of the instant invention include, without limitation: breast cancer, ovarian cancer, thyroid cancer (e.g., BRAF-mutant thyroid cancer), melanoma (e.g., BRAF- and/or RAC1-mutant malignant melanoma), malignant mesothelioma, or colon cancer). In some embodiments, the cancer is breast cancer, pancreatic cancer, ovarian cancer, brain cancer, lung cancer, colon cancer, a hematological cancer, or an intradural tumor.

The methods may further comprise the administration of at least one other cancer therapy to the subject. Examples of additional therapies include, without limitation: surgery (e.g., tumor excision), chemotherapies (chemotherapeutic agents), immunotherapies, cell therapies, targeted therapy (e.g., small molecule inhibitors, antibodies), radiosentizer, and radiation therapy (e.g., external beam radiation, ionizing radiation, radiopharmaceuticals). In some embodiments, the other cancer therapy is an inhibitory nucleic acid (e.g., siRNA, antisense, or shRNA) against PAK1. In some embodiments, the other cancer therapy is a drug efflux inhibitor or efflux pump inhibitor. The compound of the instant invention may be administered to a subject consecutively (e.g., before and/or after) and/or simultaneously with another therapy for treating, inhibiting, and/or preventing the cancer in the subject. In a particular embodiment, the compound of the instant invention is administered with at least one chemotherapeutic agent.

Chemotherapeutic agents are compounds that exhibit anticancer activity and/or are detrimental to a cell (e.g., a toxin). Suitable chemotherapeutic agents include, but are not limited to: receptor tyrosine kinase inhibitors, toxins (e.g., saporin, ricin, abrin, ethidium bromide, diptheria toxin, Pseudomonas exotoxin, and others listed above; thereby generating an immunotoxin when conjugated or fused to an antibody); alkylating agents (e.g., nitrogen mustards such as chlorambucil, cyclophosphamide, ifosamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase I inhibitor (e.g., topotecan); topoisomerase II inhibitors (e.g., amsacrine, dactinomycin, daunorubicin, idarubicin, mitoxantrone, doxorubicin, etoposide, and teniposide); DNA minor groove binding agents (e.g., plicamycin); antimetabolites (e.g., folate antagonists such as methotrexate and trimetrexate); pyrimidine antagonists (analogs) such as fluorouracil (5-fluorouracil), gemcitabine, fluorodeoxyuridine, CB3717, azacitidine, cytarabine, and floxuridine; purine antagonists (analogs) such as mercaptopurine, 6-thioguanine, fludarabine, pentostatin; asparaginase; ribonucleotide reductase inhibitors (such as hydroxyurea); tubulin interactive agents (e.g., vincristine, vinblastine, docetaxel, and paclitaxel (Taxol®)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); immunomodulator (e.g., levamisole); and antihormonal antigens (e.g., tamoxifen, antiandrogen agents such as flutamide; and antiadrenal agents such as mitotane and aminoglutethimide).

Compositions comprising at least one compound of the instant invention and at least one pharmaceutically acceptable carrier are encompassed by the instant invention. Such compositions may also be administered, in a therapeutically effective amount, to a patient in need thereof for the treatment of cancer. When an additional therapy is utilized in combination with the compound of the instant invention, the compound of the instant invention may be contained within a first composition with at least one pharmaceutically acceptable carrier and the additional therapy (e.g., chemotherapeutic agent) may be contained within a second composition with at least one pharmaceutically acceptable carrier (the carriers of the two compositions may or may not be the same). Alternatively, the composition may comprise both the compound of the instant invention and additional therapy (including a pharmaceutically acceptable carrier). Having the agents in separate compositions allows for ease of sequential and/or simultaneous administration. The instant invention also encompasses kits comprising at least one composition comprising at least one compound of the instant invention and at least one composition comprising at least one additional therapy (e.g., chemotherapeutic agent).

These and other aspects of the present invention will be further appreciated upon consideration of the following Examples, which are intended to illustrate certain particular embodiments of the invention but are not intended to limit its scope, as defined by the claims.

### EXAMPLES

### Example 1: General Methods

### Reagents

Rabbit monoclonal anti-PAK1 (Cat #2602; 1:1000), rabbit monoclonal anti-PAK2 (Cat #2608; 1:1000), rabbit anti-ERK1/2 (Cat #4695; 1:2500), rabbit anti-phospho-ERK1/2 (T202/Y204) (Cat #9101; 1:2500), rabbit anti-MEK1/2 (Cat #9122; 1:1000), rabbit anti-phospho-MEK1 (S298) (Cat #9128; 1:1000), and rabbit anti-GAPDH (Cat #2118; 1:2500) were obtained from Cell Signaling Technology. Rabbit polyclonal anti-phospho-PAK1/2/3 (S141) (Cat #44940G; 1:1000), Lipofectamine™ 3000 (Cat #L3000001), Lipofectamine™ RNAiMAX (Cat #13778100), Z-Lyte (PAK1) (PV2830), and Z-Lyte (PAK2) (PV4565) were obtained from invitrogen. DMSO (Cat #BP231-100) was obtained from Fisher. Bortezomib (PS-341) (Cat #S1013) and Lenalidomide (CC-5013) (Cat #S1029) were obtained from Selleckchem. Mouse Vinculin (V9131 1:2000), NVS-PAK1-1 (Cat #SML1867), Valspodar (Cat #SML0572), and Doxycycline Hyclate (Cat #D9891-1G) were obtained from Sigma-Aldrich. DMEM Medium (Cat #10566-016), RPMI Medium (Cat #72400-047), and Penicillin/Streptomycin (Cat #15150-122) were obtained from Gibco. Fetal Bovine Serum (Cat #SH30071.03) was obtained from HyClone. Viral Boost Reagent (Cat #VB100) was obtained from Alstem. ON-TARGETplus HUMAN PAK1 (5062) siRNA-SMARTpool (Cat #L-003521-00-005), ON-TARGETplus HUMAN PAK2 (5058) siRNA-SMARTpool (Cat #L-003597-00-0005), and ON-TARGETplus Non-Targeting Pool (Cat #D-001810-10-05) were obtained from Dharmacon. EcoRI (Cat #R3101) and XhoI (Cat #R0146) were obtained from New England Biolabs. In-Fusion HD enzyme (Cat #639649) and Stellar competent cells (Cat #636766) were obtained from Takara. pFN31K-Nluc (Cat #N1321) and Nano-Glo® Endurazine™ Live Cell Substrate (Cat #N2570/1/2) were obtained from Promega. pLenti-BFP was described in Budagyan, et al. (2021) Methods Mol. Biol., 2262:323-334). pCMV6M-Pak1 (Cat #12209) was obtained from Addgene. MOLT4, HEK293, OVCAR3, and MCF7 were obtained from ATCC. HeyA8 cells were obtained from MDACC.

### General Chemistry Methods

Analytical grade solvents and commercially available reagents were purchased from commercial sources and used directly without further purification unless otherwise stated. Thin-layer chromatography (TLC) was carried out on Merck 60 F254 precoated, glass silica plates which were visualized by ultraviolet light. Experiments were conducted under ambient conditions unless otherwise stated. ¹H-NMR, ¹³C-NMR, and ¹⁹F-NMR spectra were recorded at room temperature using a Bruker 500 (¹H-NMR at 500 MHz, ¹³C-NMR at 125 MHz, and ¹⁹F-NMR at 471 MHz). Chemical shifts are reported in ppm with reference to solvent signals [¹H-NMR: CDCl₃ (7.26 ppm), DMSO-d₆ (2.50 ppm); ¹³C-NMR: CDCl₃ (77.16 ppm), DMSO-d₆ (39.52 ppm)]. Signal patterns are indicated as s, singlet; br s, broad singlet; d, doublet; t, triplet, q, quartet; p, pentet; and m, multiplet. Mass spectrometry (MS) analysis was obtained on a Waters Acquity UPLC-MS system using electrospray ionization (ESI) in positive ion mode, reporting the molecular ion [M+H]⁺, [M+Na]⁺, or a suitable fragment ion. Flash chromatography purification was conducted using an ISCO CombiFlash® RF+ with RediSep® Rf silica cartridges. Preparative reverse-phase HPLC purification was conducted using a Waters model 2545 pump and 2489 UV/Vis detector using SunFire™ Prep C18 5 μm columns (18×100 mm, 20 mL/min flow rate; 30×250 mm, 40 mL/min flow rate), and a gradient solvent system of water (0.035% TFA)/methanol (0.035% TFA) or water (0.035% TFA)/acetonitrile (0.035% TFA).

### Abbreviations

CDI, carbonyldiimidazole; DCM, dichloromethane; DIPEA, diisopropylethylamine; DMF, N,N-dimethylformamide; DMP, dess-martin periodane; DMSO, dimethyl sulfoxide; EtOAc, ethyl acetate; HATU, hexafluorophosphate azabenzotriazole tetramethyl uronium; HPLC, high-performance liquid chromatography; MeCN, acetonitrile; MeOH, methanol; MeI, methyl iodide; Pd₂(dba)₃, tris(dibenzylideneacetone)dipalladium(0); PAK, p21-activated kinase; PROTAC, Proteolysis Targeting Chimera; TEA, triethylamine; TFA, trifluoroacetic acid; UPLC-MS, ultra-performance liquid chromatography-mass spectrometry; XPhos, 2-dicyclohexylphosphino-2′,4′,6′-triisopropylbiphenyl.

### Example 2: Experimental Procedures and Characterizations

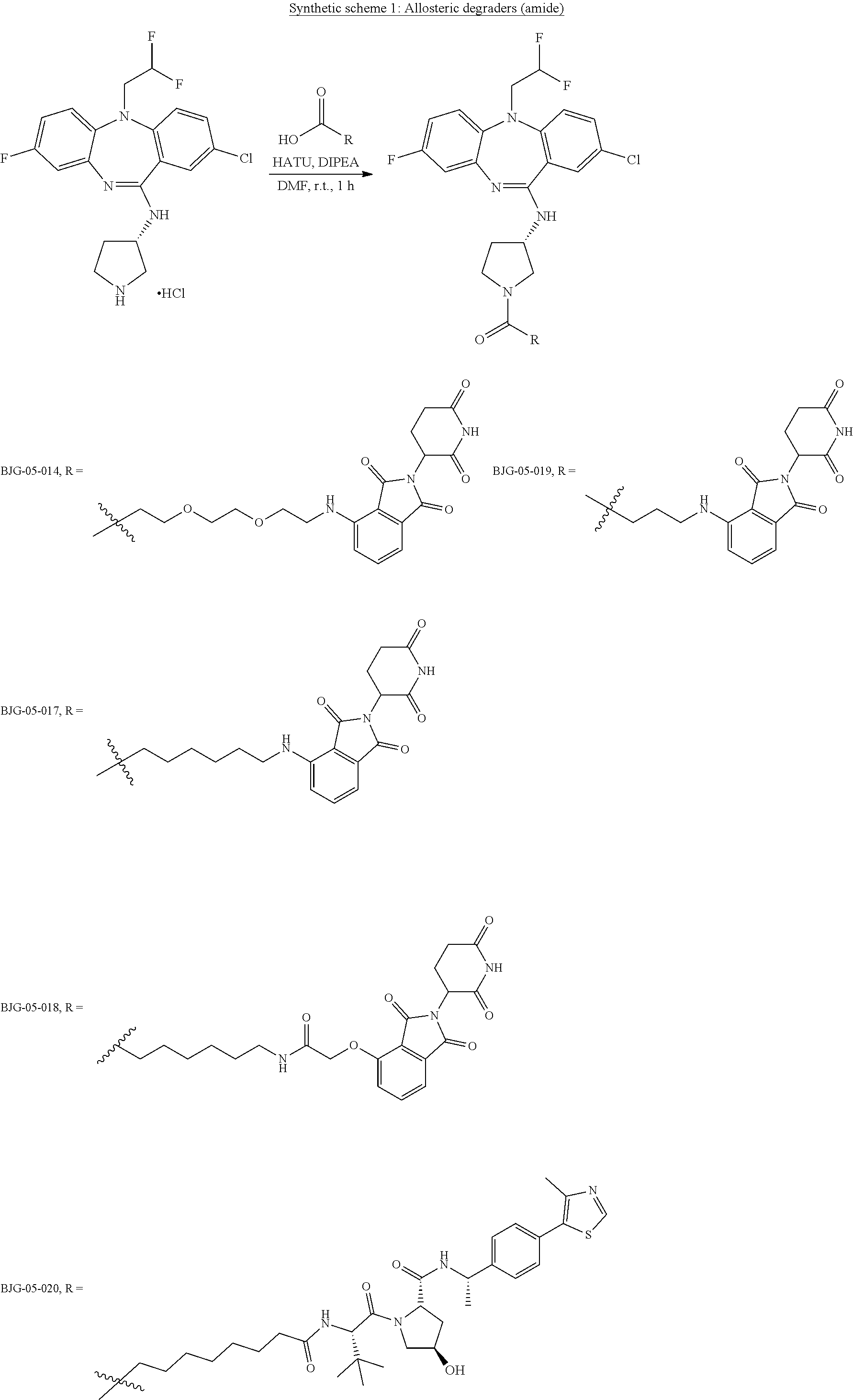

### (S)-2-Chloro-5-(2,2-difluoroethyl)-8-fluoro-N-(pyrrolidin-3-yl)-5H-dibenzo[b,e][1,4]diazepin-11-amine (HCl Salt)

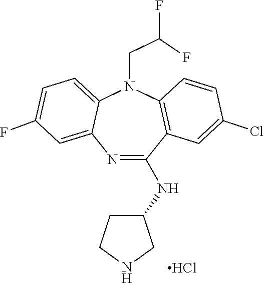 [SMILES: Fc1ccc2c(c1)N=C(N[C@H]1CCNC1)c1cc(Cl)ccc1N2CC(F)F ; Cl]

The title compound was prepared according to a known procedure (Karpov, et al. (2015) ACS Med. Chem. Lett. 6:776-781; McCoull, et al. (2016) ACS Med. Chem. Lett., 7:1118-1123).

### 4-((2-(2-(3-((S)-3-((2-Chloro-5-(2,2-difluoroethyl)-8-fluoro-5H-dibenzo[b,e][1,4]diazepin-11-yl)amino)pyrrolidin-1-yl)-3-oxopropoxy)ethoxy)ethyl)amino)-2-(2,6-dioxopiperidin-3-yl)isoindoline-1,3-dione

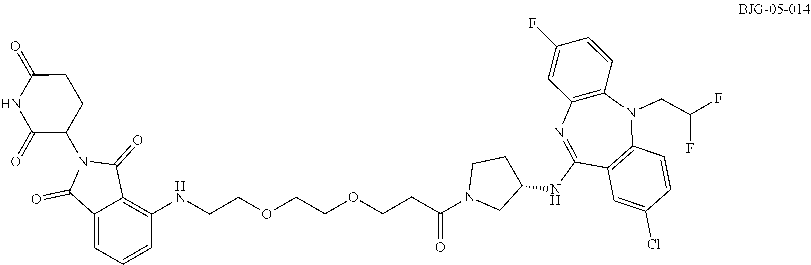 [SMILES: O=C1CCC(N2C(=O)c3cccc(NCCOCCOCCC(=O)N4CC[C@H](NC5=Nc6cc(F)ccc6N(CC(F)F)c6ccc(Cl)cc65)C4)c3C2=O)C(=O)N1]

To a solution of solution of 3-(2-(2-((2-(2,6-dioxopiperidin-3-yl)-1,3-dioxoisoindolin-4-yl)amino)ethoxy)ethoxy)propanoic acid (13.0 mg, 0.030 mmol, 1 eqiv) in N,N-dimethylformamide (0.6 mL), diisopropylamine (20.9 μL, 0.120 mmol, 4 equiv) and HATU (11.4 mg, 0.030 mmol, 1 equiv) were added. After stirring the mixture at room temperature for 5 minutes, (S)-2-chloro-5-(2,2-difluoroethyl)-8-fluoro-N-(pyrrolidin-3-yl)-5H-dibenzo[b,e][1,4]diazepin-11-amine (HCl salt) (12.9 mg, 0.030 mmol, 1 equiv) was added. After 2 hours of stirring, the reaction mixture was diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (3.8 mg, 16% yield TFA salt). ¹H NMR (500 MHz, DMSO-d₆) δ 11.09 (s, 1H), 7.79-7.39 (m, 4H), 7.32 (s, 1H), 7.14 (ddd, J=9.6, 6.2, 3.8 Hz, 1H), 7.07-6.78 (m, 3H), 6.60 (t, J=6.3 Hz, 1H), 6.03 (t, J=55.8 Hz, 1H), 5.05 (dt, J=12.8, 4.7 Hz, 1H), 4.71-4.52 (m, 1H), 4.23 (dd, J=49.1, 14.0 Hz, 2H), 3.89-3.51 (m, 14H), 2.88 (td, J=15.5, 14.2, 4.6 Hz, 1H), 2.63-2.52 (m, 2H), 2.35-2.09 (m, 1H), 2.07-1.94 (m, 2H). (Signals broadened due to rotational isomerism). LRMS (ESI) calculated for [M+H]⁺ 810.26. found 809.71.

### 4-((7-((S)-3-((2-Chloro-5-(2,2-difluoroethyl)-8-fluoro-5H-dibenzo[b,e][1,4]diazepin-11-yl)amino)pyrrolidin-1-yl)-7-oxoheptyl)amino)-2-(2,6-dioxopiperidin-3-yl)isoindoline-1,3-dione

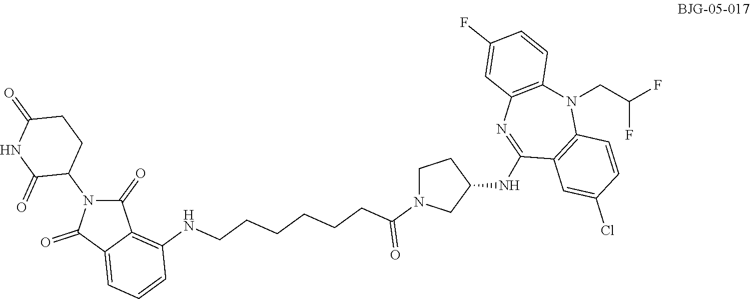 [SMILES: O=C1CCC(N2C(=O)c3cccc(NCCCCCCC(=O)N4CC[C@H](NC5=Nc6cc(F)ccc6N(CC(F)F)c6ccc(Cl)cc65)C4)c3C2=O)C(=O)N1]

To a solution of solution of 7-((2-(2,6-dioxopiperidin-3-yl)-1,3-dioxoisoindolin-4-yl)amino)heptanoic acid (12.0 mg, 0.030 mmol, 1 equiv) in N,N-dimethylformamide (0.6 mL), diisopropylamine (20.9 μL, 0.120 mmol, 4 equiv) and HATU (11.4 mg, 0.030 mmol, 1 equiv) were added. After stirring the mixture at room temperature for 5 minutes, (S)-2-chloro-5-(2,2-difluoroethyl)-8-fluoro-N-(pyrrolidin-3-yl)-5H-dibenzo[b,e][1,4]diazepin-11-amine (HCl salt) (12.9 mg, 0.030 mmol, 1 equiv) was added. After 2 hours of stirring, the reaction mixture was diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (2.1 mg, 9% yield TFA salt). ¹H NMR (500 MHz, DMSO-d₆) δ 11.08 (s, 1H), 7.62-7.44 (m, 3H), 7.43-7.26 (m, 2H), 7.17-7.03 (m, 2H), 7.01 (d, J=7.0 Hz, 1H), 6.77-6.61 (m, 2H), 6.52 (p, J=6.0 Hz, 1H), 5.97 (tt, J=55.7, 3.8 Hz, 1H), 5.11-4.98 (m, 1H), 4.63-4.46 (m, 1H), 4.25-3.98 (m, 2H), 3.75-3.59 (m, 1H), 3.58-3.34 (m, 5H), 2.88 (ddd, J=17.4, 13.6, 5.4 Hz, 1H), 2.64-2.52 (m, 2H), 2.31-1.92 (m, 5H), 1.64-1.42 (m, 5H), 1.41-1.16 (m, 7H). 6:1 mix of rotamers. LRMS (ESI) calculated for [M+H]⁺ 778.26. found 777.71.

### N-(7-((S)-3-((2-Chloro-5-(2,2-difluoroethyl)-8-fluoro-5H-dibenzo[b,e][1,4]diazepin-11-yl)amino)pyrrolidin-1-yl)-7-oxoheptyl)-2-((2-(2,6-dioxopiperidin-3-yl)-1,3-dioxoisoindolin-4-yl)oxy)acetamide

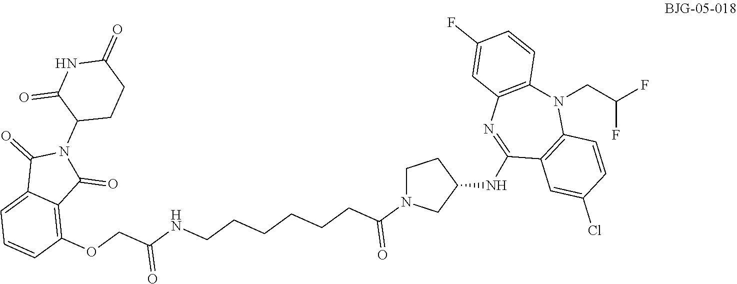 [SMILES: O=C(COc1cccc2c1C(=O)N(C1CCC(=O)NC1=O)C2=O)NCCCCCCC(=O)N1CC[C@H](NC2=Nc3cc(F)ccc3N(CC(F)F)c3ccc(Cl)cc32)C1]

To a solution of solution of 7-(2-((2-(2,6-dioxopiperidin-3-yl)-1,3-dioxoisoindolin-4-yl)oxy)acetamido)heptanoic acid (13.8 mg, 0.030 mmol, 1 equiv) in N,N-dimethylformamide (0.6 mL), diisopropylamine (20.9 μL, 0.120 mmol, 4 equiv) and HATU (11.4 mg, 0.030 mmol, 1 equiv) were added. After stirring the mixture at room temperature for 5 minutes, (S)-2-chloro-5-(2,2-difluoroethyl)-8-fluoro-N-(pyrrolidin-3-yl)-5H-dibenzo[b,e][1,4]diazepin-11-amine (HCl salt) (12.9 mg, 0.030 mmol, 1 equiv) was added. After 2 hours of stirring, the reaction mixture was diluted with 1.0 mL of N,N-dimethylformamide and purified by reverse-phase prep HPLC (95-15% H₂O/MeOH, 40 m/min, 45 min). Lyophilization from H₂O/MeCN provided the title compound as a yellow powder (2.5 mg, 10% yield TFA salt). ¹H NMR (500 MHz, DMSO-d₆) δ 11.11 (s, 1H), 7.97-7.88 (m, 1H), 7.85-7.78 (m, 1H), 7.61-7.46 (m, 3H), 7.42-7.28 (m, 3H), 7.11 (t, J=7.7 Hz, 1H), 6.77-6.62 (m, 2H), 5.99 (tt, J=55.1, 3.8 Hz, 1H), 5.12 (ddd, J=12.6, 5.4, 2.2 Hz, 1H), 4.77 (s, 2H), 4.65-4.46 (m, 1H), 4.26-3.97 (m, 2H), 3.75-3.59 (m, 1H), 3.57-3.35 (m, 5H), 3.12 (dq, J=13.2, 6.3 Hz, 2H), 2.90 (ddd, J=19.0, 13.7, 5.4 Hz, 1H), 2.64-2.52 (m, 2H), 2.30-1.94 (m, 5H), 1.56-1.35 (m, 4H), 1.34-1.21 (m, 5H). LRMS (ESI) calculated for [M+H]⁺ 836.27. found 835.61.

### 4-((4-((S)-3-((2-Chloro-5-(2,2-difluoroethyl)-8-fluoro-5H-dibenzo[b,e][1,4]diazepin-11-yl)amino)pyrrolidin-1-yl)-4-oxobutyl)amino)-2-(2,6-dioxopiperidin-3-yl)isoindoline-1,3-dione

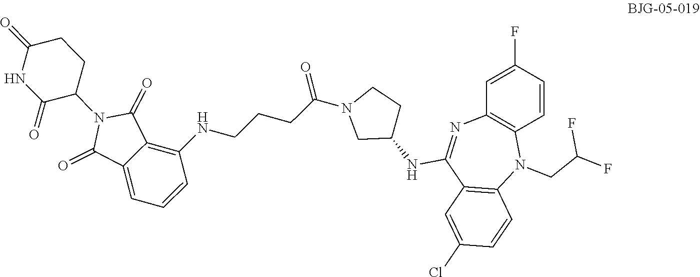 [SMILES: O=C1CCC(N2C(=O)c3cccc(NCCCC(=O)N4CC[C@H](NC5=Nc6cc(F)ccc6N(CC(F)F)c6ccc(Cl)cc65)C4)c3C2=O)C(=O)N1]

To a solution of solution of 4-((2-(2,6-dioxopiperidin-3-yl)-1,3-dioxoisoindolin-4-yl)amino)butanoic acid (14.4 mg, 0.040 mmol, 1 equiv) in N,N-dimethylformamide (0.8 mL), diisopropylamine (27.9 μL, 0.160 mmol, 4 equiv) and HATU (15.2 mg, 0.040 mmol, 1 equiv) were added. After stirring the mixture at room temperature for 5 minutes, (S)-2-chloro-5-(2,2-difluoroethyl)-8-fluoro-N-(pyrrolidin-3-yl)-5H-dibenzo[b,e][1,4]diazepin-11-amine (HCl salt) (17.2 mg, 0.040 mmol, 1 equiv) was added. After 2 hours of stirring, the reaction mixture was diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (4.1 mg, 14% yield TFA salt). ¹H NMR (500 MHz, DMSO-d₆) δ 11.09 (s, 1H), 7.80-7.29 (m, 5H), 7.23-7.15 (m, 1H), 7.14-6.97 (m, 2H), 6.68 (s, 1H), 6.09 (t, J=55.0 Hz, 1H), 5.04 (dt, J=12.9, 5.3 Hz, 1H), 4.65 (d, J=26.9 Hz, 1H), 4.41-4.12 (m, 2H), 3.41-3.28 (m, 2H), 2.96-2.78 (m, 1H), 2.64-2.52 (m, 2H), 2.44-1.96 (m, 5H), 1.90-1.74 (m, 2H). (Signals broadened due to rotational isomerism). LRMS (ESI) calculated for [M+H]⁺ 736.21. found 735.71.

### (2S,4R)-1-((S)-2-(9-((S)-3-((2-Chloro-5-(2,2-difluoroethyl)-8-fluoro-5H-dibenzo[b,e][1,4]diazepin-11-yl)amino)pyrrolidin-1-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide

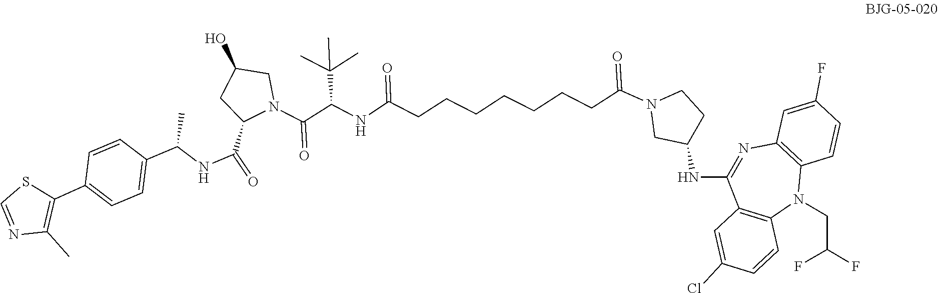 [SMILES: Cc1ncsc1-c1ccc([C@H](C)NC(=O)[C@@H]2C[C@@H](O)CN2C(=O)[C@@H](NC(=O)CCCCCCCC(=O)N2CC[C@H](NC3=Nc4cc(F)ccc4N(CC(F)F)c4ccc(Cl)cc43)C2)C(C)(C)C)cc1]

To a solution of solution of 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic acid (9.2 mg, 0.015 mmol, 1 equiv) in N,N-dimethylformamide (0.3 mL), diisopropylamine (10.5 μL, 0.060 mmol, 4 equiv) and HATU (5.7 mg, 0.015 mmol, 1 equiv) were added. After stirring the mixture at room temperature for 5 minutes, (S)-2-chloro-5-(2,2-difluoroethyl)-8-fluoro-N-(pyrrolidin-3-yl)-5H-dibenzo[b,e][1,4]diazepin-11-amine (HCl salt) (6.5 mg, 0.015 mmol, 1 equiv) was added. After 2 hours of stirring, the reaction mixture was diluted with 1.0 mL of N,N-dimethylformamide and purified by reverse-phase prep HPLC (95-5% H₂O/MeOH, 40 mL/min, 45 min). Lyophilization from H₂O/MeCN provided the title compound as a white powder (2.7 mg, 18% yield TFA salt). ¹H NMR (500 MHz, DMSO-d₆) δ 8.98 (s, 1H), 8.37 (d, J=8.0 Hz, 1H), 7.77 (d, J=9.1 Hz, 1H), 7.74-7.46 (m, 3H), 7.46-7.30 (m, 5H), 7.26-6.91 (m, 1H), 6.10 (t, J=55.3 Hz, 1H), 4.91 (p, J=7.0 Hz, 1H), 4.73-4.58 (m, 1H), 4.51 (dd, J=9.3, 5.2 Hz, 1H), 4.42 (t, J=8.1 Hz, 1H), 3.63-3.55 (m, 4H), 2.45 (s, 4H), 2.31-1.94 (m, 7H), 1.83-1.75 (m, 1H), 1.56-1.40 (m, 5H), 1.37 (d, J=6.9 Hz, 3H), 1.32-1.16 (m, 7H), 0.93 (s, 9H). LRMS (ESI) calculated for [M+H]⁺ 991.42. found 990.73.

### Synthetic Scheme 2: Allosteric Degraders (Urea)

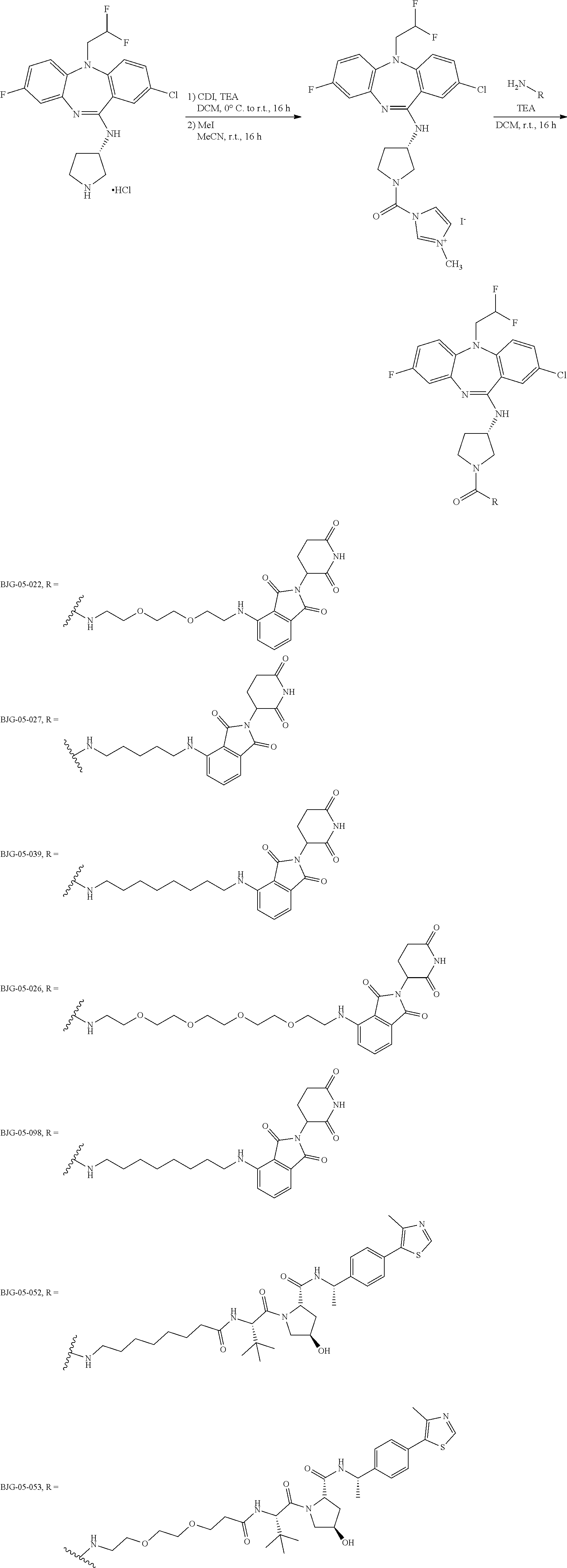

### (S)-1-(3-((2-Chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium Iodide

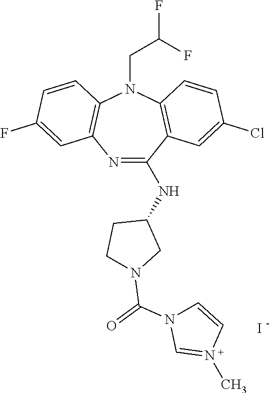

A solution of carbonyldiimidazole (50 mg, 0.31 mmol, 1.1 equiv) in dichloromethane (1.4 mL) was cooled to 0° C. at which (S)-2-chloro-5-(2,2-difluoroethyl)-8-fluoro-N-(pyrrolidin-3-yl)-5H-dibenzo[b,e][1,4]diazepin-11-amine (HCl salt) (120.8 mg, 0.28 mmol, 1.0 equiv) was added followed by addition of triethylamine (39 μL, 0.28 mmol, 1 equiv). The mixture was then removed from ice and allowed to stir at room temperature overnight. The reaction was diluted with 7 mL of water and organic layer was then extracted with dichloromethane (5×2 mL). The combined organic layers were washed with brine, dried over MgSO₄, filtered, and concentrated to provide a clear yellow oil (60 mg). Product was shown as the major component via mass spec: LRMS (ESI) calculated for [M+H]⁺ 489.13. found 488.90. The crude product was moved forward without purification.

To a solution of 50 mg of crude residue in acetonitrile (1.4 mL), iodomethane (70 μL, 1.12 mmol, 4 equiv) was added at room temperature and stirred overnight. The reaction mixture was concentrated to provide the titled compound as a yellow oil (62.5 mg, 99% yield). The product was shown as the major component via mass spec, LRMS (ESI) calculated for [M+H]⁺ 504.16. found 502.78, and moved on to the next step without purification.

### (3S)-3-((2-Chloro-5-(2,2-difluoroethyl)-8-fluoro-5H-dibenzo[b,e][1,4]diazepin-11-yl)amino)-N-(2-(2-(2-((2-(2,6-dioxopiperidin-3-yl)-1,3-dioxoisoindolin-4-yl)amino)ethoxy)ethoxy)ethyl)pyrrolidine-1-carboxamide

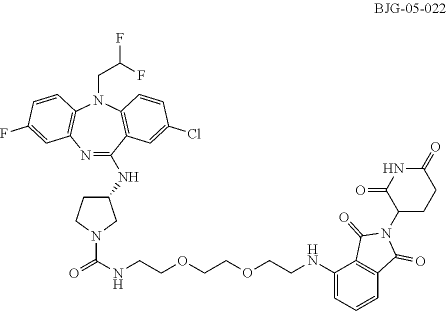 [SMILES: O=C1CCC(N2C(=O)c3cccc(NCCOCCOCCNC(=O)N4CC[C@H](NC5=Nc6cc(F)ccc6N(CC(F)F)c6ccc(Cl)cc65)C4)c3C2=O)C(=O)N1]

To a solution of solution of 4-((2-(2-(2-aminoethoxy)ethoxy)ethyl)amino)-2-(2,6-dioxopiperidin-3-yl)isoindoline-1,3-dione (15.6 mg, 0.030 mmol, 1 equiv) and (S)-1-(3-((2-chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium iodide (18.9 mg, 0.030 mmol, 1 equiv) in dichloromethane (0.3 mL), triethylamine (10.5 μL, 0.075 mmol, 2.5 equiv) was added. After 16 hours of stirring, the reaction mixture was concentrated and diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (5.1 mg, 21% yield TFA salt). ¹H NMR (500 MHz, DMSO-d₆) δ 11.09 (s, 1H), 7.69 (s, 1H), 7.58 (ddd, J=8.5, 7.0, 1.5 Hz, 2H), 7.49 (s, 1H), 7.39 (s, 1H), 7.27-6.98 (m, 4H), 6.60 (s, 1H), 6.33-5.89 (m, 2H), 5.06 (dd, J=12.6, 5.4 Hz, 1H), 4.60 (s, 1H), 4.40-4.14 (m, 2H), 4.10 (d, J=1.1 Hz, 1H), 3.57-3.29 (m, 12H), 3.18 (dq, J=12.1, 6.1 Hz, 2H), 2.88 (ddd, J=16.7, 13.6, 5.4 Hz, 1H), 2.62-2.52 (m, 2H), 2.34-1.95 (m, 3H). LRMS (ESI) calculated for [M+H]⁺ 825.26. found 824.71.

### (3S)-3-((2-Chloro-5-(2,2-difluoroethyl)-8-fluoro-5H-dibenzo[b,e][1,4]diazepin-11-yl)amino)-N-(14-((2-(2,6-dioxopiperidin-3-yl)-1,3-dioxoisoindolin-4-yl)amino)-3,6,9,12-tetraoxatetradecyl)pyrrolidine-1-carboxamide

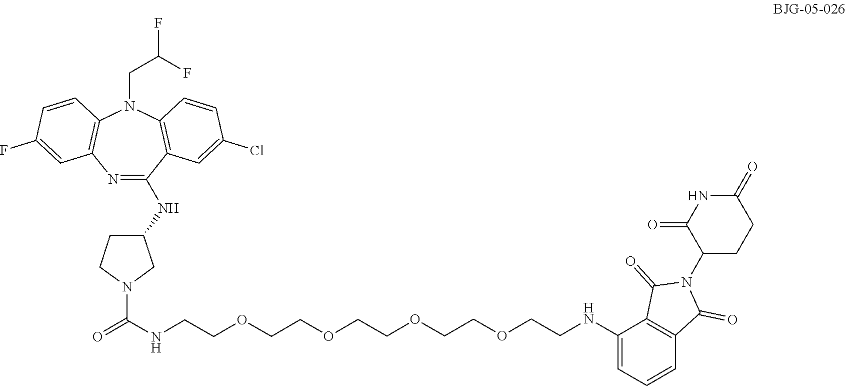 [SMILES: O=C1CCC(N2C(=O)c3cccc(NCCOCCOCCOCCOCCNC(=O)N4CC[C@H](NC5=Nc6cc(F)ccc6N(CC(F)F)c6ccc(Cl)cc65)C4)c3C2=O)C(=O)N1]

To a solution of solution of 4-((14-amino-3,6,9,12-tetraoxatetradecyl)amino)-2-(2,6-dioxopiperidin-3-yl)isoindoline-1,3-dione (23 mg, 0.030 mmol, 1 equiv) and (S)-1-(3-((2-chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium iodide (18.9 mg, 0.030 mmol, 1 equiv) in dichloromethane (0.3 mL), triethylamine (10.5 μL, 0.075 mmol, 2.5 equiv) was added. After 16 hours of stirring, the reaction mixture was concentrated and diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (4.8 mg, 18% yield TFA salt). ¹H NMR (500 MHz, DMSO-d₆) δ 11.09 (s, 1H), 7.67-7.46 (m, 3H), 7.44-7.24 (m, 2H), 7.18-7.07 (m, 2H), 7.04 (d, J=7.0 Hz, 1H), 6.79-6.63 (m, 2H), 6.60 (t, J=5.9 Hz, 1H), 6.21-5.82 (m, 2H), 5.05 (dd, J=12.7, 5.5 Hz, 1H), 4.53 (t, J=4.9 Hz, 1H), 4.28-3.93 (m, 2H), 3.64-3.34 (m, 20H), 3.24-3.10 (m, 2H), 2.88 (ddd, J=16.7, 13.7, 5.4 Hz, 1H), 2.62-2.51 (m, 2H), 2.24-1.87 (m, 3H). LRMS (ESI) calculated for [M+H]⁺ 913.31. found 912.62.

### (3S)-3-((2-Chloro-5-(2,2-difluoroethyl)-8-fluoro-5H-dibenzo[b,e][1,4]diazepin-11-yl)amino)-N-(5-((2-(2,6-dioxopiperidin-3-yl)-1,3-dioxoisoindolin-4-yl)amino)pentyl)pyrrolidine-1-carboxamide

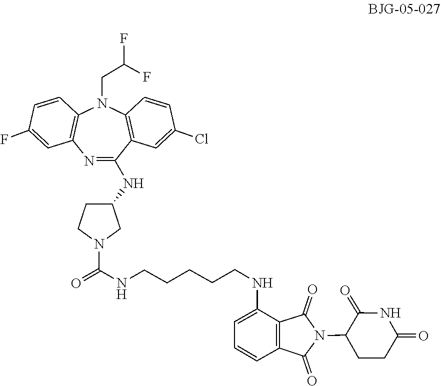 [SMILES: O=C1CCC(N2C(=O)c3cccc(NCCCCCNC(=O)N4CC[C@H](NC5=Nc6cc(F)ccc6N(CC(F)F)c6ccc(Cl)cc65)C4)c3C2=O)C(=O)N1]

To a solution of solution of 4-((5-aminopentyl)amino)-2-(2,6-dioxopiperidin-3-yl)isoindoline-1,3-dione (15.1 mg, 0.030 mmol, 1 equiv) and (S)-1-(3-((2-chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium iodide (18.9 mg, 0.030 mmol, 1 equiv) in dichloromethane (0.3 mL), triethylamine (10.5 μL, 0.075 mmol, 2.5 equiv) was added. After 16 hours of stirring, the reaction mixture was concentrated and diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (3.8 mg, 16% yield TFA salt). ¹H NMR (500 MHz, DMSO-d₆) δ 11.09 (s, 1H), 7.70 (s, 1H), 7.65-7.54 (m, 2H), 7.53-7.29 (m, 2H), 7.12-6.98 (m, 4H), 6.92 (dq, J=7.5, 0.8 Hz, 1H), 6.57-6.41 (m, 2H), 6.30-5.92 (m, 2H), 5.05 (ddd, J=12.8, 5.6, 1.3 Hz, 1H), 4.67 (s, 1H), 4.33 (s, 4H), 3.74-3.64 (m, 1H), 3.36 (dd, J=8.7, 5.8 Hz, 2H), 3.31-3.24 (m, 2H), 3.14-2.97 (m, 4H), 2.88 (ddd, J=16.8, 13.8, 5.4 Hz, 1H), 2.62-2.52 (m, 2H), 2.02 (ddd, J=13.1, 6.5, 4.2 Hz, 2H), 1.58 (h, J=7.0 Hz, 2H), 1.45 (dq, J=11.3, 7.2 Hz, 2H), 1.34 (dq, J=15.6, 8.0, 7.0 Hz, 2H). LRMS (ESI) calculated for [M+H]⁺ 779.26. found 778.71.

### (3S)-3-((2-Chloro-5-(2,2-difluoroethyl)-8-fluoro-5H-dibenzo[b,e][1,4]diazepin-11-yl)amino)-N-(8-((2-(2,6-dioxopiperidin-3-yl)-1,3-dioxoisoindolin-4-yl)amino)octyl)pyrrolidine-1-carboxamide

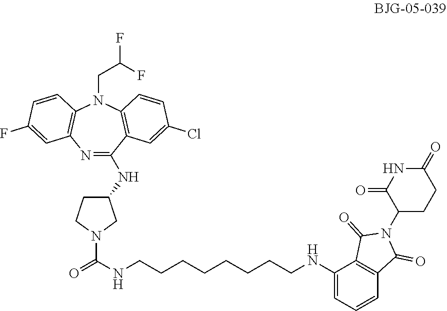 [SMILES: O=C1CCC(N2C(=O)c3cccc(NCCCCCCCCNC(=O)N4CC[C@H](NC5=Nc6cc(F)ccc6N(CC(F)F)c6ccc(Cl)cc65)C4)c3C2=O)C(=O)N1]

To a solution of solution of 4-((8-aminooctyl)amino)-2-(2,6-dioxopiperidin-3-yl)isoindoline-1,3-dione (20.4 mg, 0.040 mmol, 1 equiv) and (S)-1-(3-((2-chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium iodide (25.2 mg, 0.040 mmol, 1 equiv) in dichloromethane (0.5 mL), triethylamine (13.9 μL, 0.100 mmol, 2.5 equiv) was added. After 16 hours of stirring, the reaction mixture was concentrated and diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (7.8 mg, 24% yield TFA salt). ¹H NMR (500 MHz, DMSO-d₆) δ 11.09 (s, 1H), 7.71 (d, J=9.2 Hz, 1H), 7.64-7.49 (m, 3H), 7.48-7.35 (m, 1H), 7.16-7.08 (m, 2H), 7.02 (d, J=7.0 Hz, 1H), 6.51 (s, 1H), 6.25-5.96 (m, 2H), 5.05 (dd, J=12.7, 5.4 Hz, 1H), 4.68 (s, 1H), 4.44-4.12 (m, 2H), 3.68 (ddd, J=14.0, 11.1, 6.1 Hz, 1H), 3.39-3.31 (m, 2H), 3.28 (t, J=7.5 Hz, 3H), 3.05-2.95 (m, 2H), 2.88 (ddd, J=16.8, 13.7, 5.4 Hz, 1H), 2.64-2.52 (m, 2H), 2.39-2.15 (m, 2H), 2.09-1.96 (m, 2H), 1.61-1.51 (m, 2H), 1.45-1.21 (m, 10H). LRMS (ESI) calculated for [M+H]⁺ 821.21. found 821.15.

### (S)-3-((2-Chloro-5-(2,2-difluoroethyl)-8-fluoro-5H-dibenzo[b,e][1,4]diazepin-11-yl)amino)-N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-J-oxobutan-2-yl)amino)-8-oxooctyl)pyrrolidine-1-carboxamide

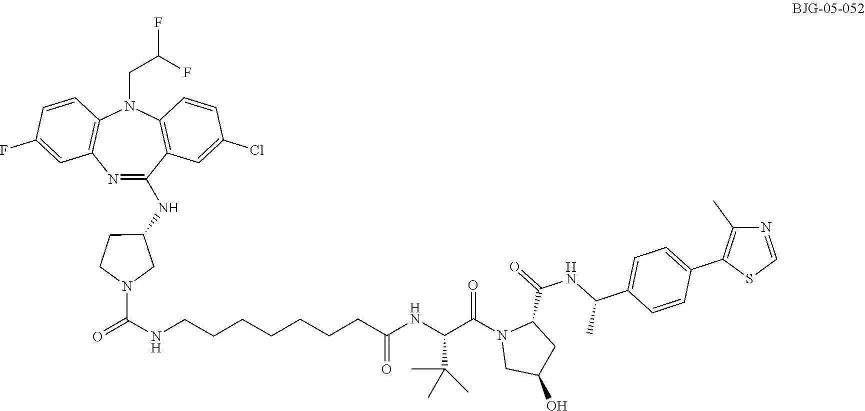 [SMILES: Cc1ncsc1-c1ccc([C@H](C)NC(=O)[C@@H]2C[C@@H](O)CN2C(=O)[C@@H](NC(=O)CCCCCCCNC(=O)N2CC[C@H](NC3=Nc4cc(F)ccc4N(CC(F)F)c4ccc(Cl)cc43)C2)C(C)(C)C)cc1]

To a solution of solution of (2S,4R)-1-((S)-2-(8-aminooctanamido)-3,3-dimethylbutanoyl)-4-hydroxy-N—((S)-1-(4-(4-methylthiazol-5-yl)phenyl)ethyl) pyrrolidine-2-carboxamide (20.9 mg, 0.030 mmol, 1 equiv) and (S)-1-(3-((2-chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium iodide (18.9 mg, 0.030 mmol, 1 equiv) in dichloromethane (0.3 mL), triethylamine (10.5 μL, 0.075 mmol, 2.5 equiv) was added. After 16 hours of stirring, the reaction mixture was concentrated and diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a white powder (4.3 mg, 14% yield TFA salt). ¹H NMR (500 MHz, DMSO-d₆) δ 8.98 (s, 1H), 8.37 (dd, J=7.9, 1.6 Hz, 1H), 7.77 (dd, J=9.3, 1.9 Hz, 1H), 7.66-7.50 (m, 2H), 7.47-7.31 (m, 6H), 6.77 (s, 1H), 6.23-5.81 (m, 2H), 5.09 (s, 1H), 4.91 (p, J=7.1 Hz, 1H), 4.56 (s, 1H), 4.51 (dd, J=9.4, 2.6 Hz, 1H), 4.42 (td, J=8.0, 1.8 Hz, 1H), 4.35-3.98 (m, 3H), 3.72-3.53 (m, 3H), 3.52-3.43 (m, 1H), 2.99 (dtd, J=17.5, 11.4, 10.9, 7.3 Hz, 2H), 2.45 (d, J=0.9 Hz, 3H), 2.29-1.94 (m, 5H), 1.79 (ddd, J=12.9, 8.5, 4.7 Hz, 1H), 1.56-1.32 (m, 8H), 1.23 (t, J=6.6 Hz, 8H), 0.93 (s, 9H). LRMS (ESI) calculated for [M+H]⁺ 1006.43. found 1005.73.

### (S)-3-((2-Chloro-5-(2,2-difluoroethyl)-8-fluoro-5H-dibenzo[b,e][1,4]diazepin-11-yl)amino)-N-(2-(2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-J-oxobutan-2-yl)amino)-3-oxopropoxy)ethoxy)ethyl)pyrrolidine-1-carboxamide

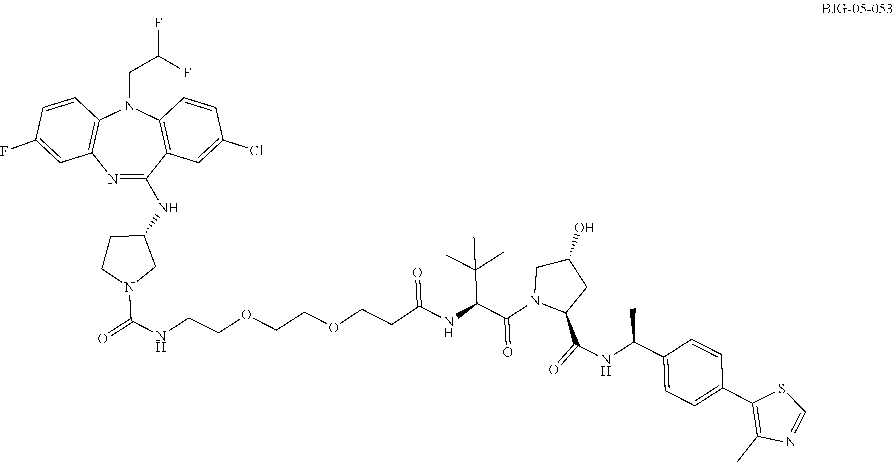 [SMILES: Cc1ncsc1-c1ccc([C@H](C)NC(=O)[C@@H]2C[C@@H](O)CN2C(=O)[C@@H](NC(=O)CCOCCOCCNC(=O)N2CC[C@H](NC3=Nc4cc(F)ccc4N(CC(F)F)c4ccc(Cl)cc43)C2)C(C)(C)C)cc1]

To a solution of solution of (2S,4R)-1-((S)-2-(3-(2-(2-aminoethoxy)ethoxy) propanamido)-3,3-dimethylbutanoyl)-4-hydroxy-N—((S)-1-(4-(4-methylthiazol-5-yl)phenyl)ethyl)pyrrolidine-2-carboxamide (21.5 mg, 0.030 mmol, 1 equiv) and (S)-1-(3-((2-chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium iodide (18.9 mg, 0.030 mmol, 1 equiv) in dichloromethane (0.3 mL), triethylamine (10.5 μL, 0.075 mmol, 2.5 equiv) was added. After 16 hours of stirring, the reaction mixture was concentrated and diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a white powder (4.9 mg, 16% yield TFA salt). ¹H NMR (500 MHz, DMSO-d₆) δ 8.98 (d, J=1.1 Hz, 1H), 8.38 (d, J=7.7 Hz, 1H), 7.86 (dd, J=9.3, 1.9 Hz, 1H), 7.66 (s, 1H), 7.60-7.26 (m, 7H), 6.33-5.87 (m, 2H), 4.91 (p, J=7.0 Hz, 1H), 4.60 (s, 1H), 4.53 (dd, J=9.3, 2.1 Hz, 1H), 4.42 (t, J=8.1 Hz, 1H), 4.37-4.10 (m, 3H), 3.64-3.57 (m, 6H), 3.43-3.26 (m, 7H), 3.22-3.12 (m, 2H), 2.45 (d, J=1.2 Hz, 3H), 2.40-2.09 (m, 3H), 2.06-1.95 (m, 2H), 1.79 (ddd, J=12.9, 8.5, 4.7 Hz, 1H), 1.37 (d, J=7.0 Hz, 3H), 0.93 (s, 9H). (PEG and pyrrolidine methylene protons obscured by water peak). LRMS (ESI) calculated for [M+H]⁺ 1024.41. found 1023.79.

### (3S)-3-((2-Chloro-5-(2,2-difluoroethyl)-8-fluoro-5H-dibenzo[b,e][1,4]diazepin-11-yl)amino)-N-(8-((2-(J-methyl-2,6-dioxopiperidin-3-yl)-1,3-dioxoisoindolin-4-yl)amino)octyl)pyrrolidine-1-carboxamide

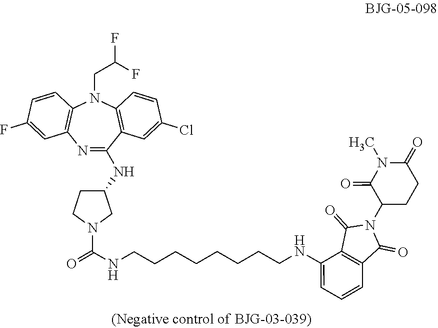 [SMILES: CN1C(=O)CCC(N2C(=O)c3cccc(NCCCCCCCCNC(=O)N4CC[C@H](NC5=Nc6cc(F)ccc6N(CC(F)F)c6ccc(Cl)cc65)C4)c3C2=O)C1=O]

To a solution of solution of 4-((8-aminooctyl)amino)-2-(1-methyl-2,6-dioxopiperidin-3-yl)isoindoline-1,3-dione (17.4 mg, 0.030 mmol, 1 equiv) and (S)-1-(3-((2-chloro-5-(2,2-difluoroethyl)-8-fluoro-5H-dibenzo[b,e][1,4]diazepin-11-yl)amino)pyrrolidine-1-carbonyl)-3-methyl-1H-imidazol-3-ium iodide (18.9 mg, 0.030 mmol, 1 equiv) in dichloromethane (0.3 mL), triethylamine (10.5 μL, 0.075 mmol, 2.5 equiv) was added. After 16 hours of stirring, the reaction mixture was concentrated and diluted with 1.0 mL of N,N-dimethylformamide and purified by reverse-phase prep HPLC (95-15% H₂O/MeOH, 40 mL/min, 45 min). Lyophilization from H₂O/MeCN provided the title compound as a yellow powder (7.1 mg, 28% yield TFA salt). ¹H NMR (500 MHz, DMSO-d₆) δ 7.61-7.45 (m, 3H), 7.39-7.28 (m, 2H), 7.13-7.05 (m, 2H), 7.02 (d, J=7.0 Hz, 1H), 6.76-6.62 (m, 2H), 6.52 (t, J=5.9 Hz, 1H), 6.12-5.83 (m, 2H), 5.11 (dt, J=13.1, 5.3 Hz, 1H), 4.51 (td, J=10.4, 9.6, 4.8 Hz, 1H), 4.26-3.95 (m, 2H), 3.60 (ddd, J=27.3, 10.5, 6.3 Hz, 1H), 3.49-3.33 (m, 1H), 3.29-3.16 (m, 3H), 3.01 (s, 3H), 3.00-2.89 (m, 2H), 2.75 (ddd, J=17.1, 4.5, 2.6 Hz, 1H), 2.59-2.52 (m, 1H), 2.22-1.90 (m, 3H), 1.55 (q, J=7.3 Hz, 2H), 1.45-1.14 (m, 12H). LRMS (ESI) calculated for [M+H]⁺ 835.32. found 834.71.

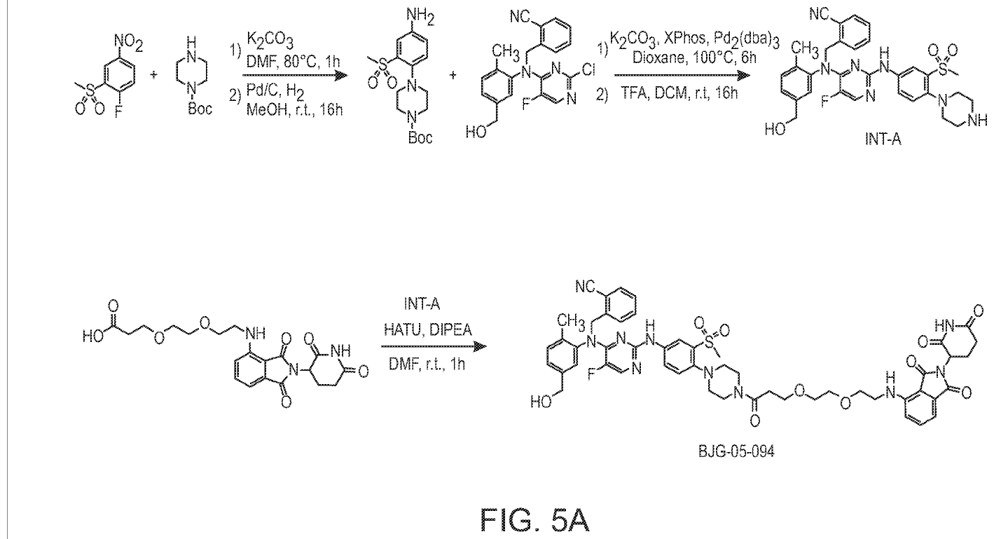

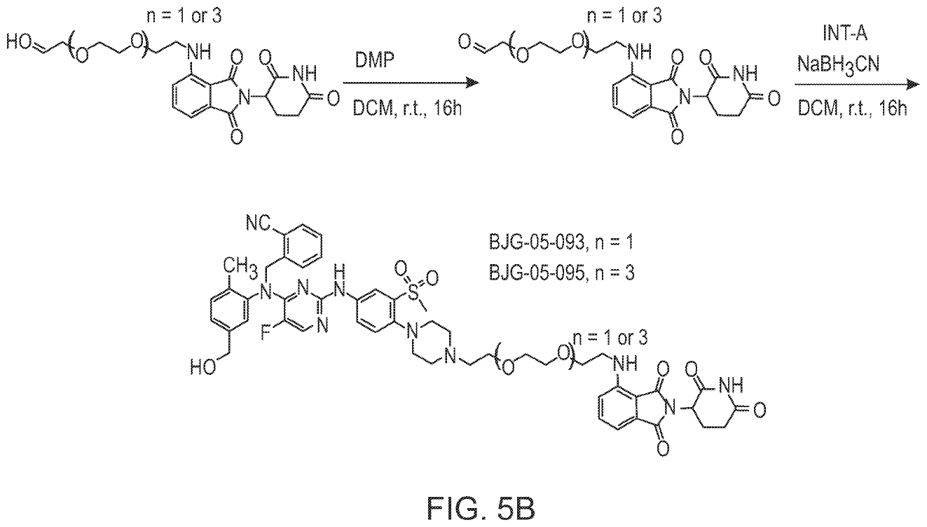

FIG. 5A and FIG. 5B provide a synthetic scheme for ATP-competitive degraders.

### tert-Butyl 4-(4-min-2-(methylsulfonyl)phenyl)piperazine-1-carboxylate

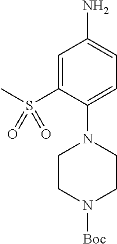 [SMILES: CC(C)(C)OC(=O)N1CCN(c2ccc(N)cc2S(C)(=O)=O)CC1]

A suspension of 1-fluoro-2-(methylsulfonyl)-4-nitrobenzene (767 mg, 3.5 mmol, 1.0 equiv), tert-butyl piperazine-1-carboxylate (1.956 g, 10.5 mmol, 3.0 equiv), and potassium carbonate (967 mg, 7.0 mmol, 2.0 equiv) in N,N-dimethylformamide (7.0 ml) was stirred at 80° C. for 1 hour. The reaction mixture was diluted with ethyl acetate (100 ml), washed with 0.5M HCl, followed by H₂O (3×). The organic fractions were combined, washed with brined, dried over MgSO₄, filtered and concentrated in vacuo, yielding 1.355 g of yellow solid (˜93% purity by NMR). To a suspension of the crude solids in methanol (25 mL), 10 wt % Pd/C (355 mg, 0.35 mmol, 0.1 equiv) was added. The reaction mixture was sparged with H2 gas for 7 minutes then stirred at room temperature for 16 hours. The mixture was filtered through celite, washing with MeOH (50 mL), and filtrate was concentrated to afford the title compound as a light brown powder (1.248 g, 99% yield, >90% purity by UPLC-MS). LRMS: [M+H]+ found 355.97

### 2-(((5-Fluoro-2-((3-(methylsulfonyl)-4-(piperazin-1-yl)phenyl)amino)pyrimidin-4-yl)(5-(hydroxymethyl)-2-methylphenyl)amino)methyl)benzonitrile

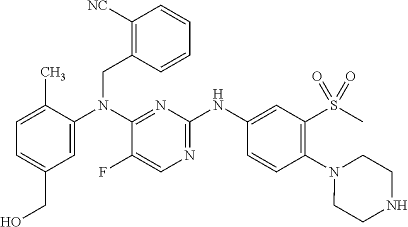 [SMILES: Cc1ccc(CO)cc1N(Cc1ccccc1C#N)c1nc(Nc2ccc(N3CCNCC3)c(S(C)(=O)=O)c2)ncc1F]

A suspension of 2-(((2-chloro-5-fluoropyrimidin-4-yl)(5-(hydroxymethyl)-2-methylphenyl)amino)methyl)benzonitrile (synthesized according to literature)(765 mg, 2.0 mmol, 1 equiv), tert-butyl 4-(4-amino-2-(methylsulfonyl)phenyl)piperazine-1-carboxylate (781 mg, 2.2 mmol, 1.1 equiv), Tris(dibenzylideneacetone)dipalladium(0) (91.6 mg, 0.10 mmol, 0.05 equiv), XPhos (95.3 mg, 0.20 mmol, 0.10 equiv), and potassium carbonate (829 mg, 6.0 mmol, 3.0 equiv) in dioxane (20 mL) was sparged with N2 for 10 minutes. The reaction mixture was then stirred at 100° C. for 6 hours. After cooling to room temperature, the mixture was filtered through Celite®, washing with ethyl acetate (50 mL), and concentrated in vacuo. The crude material was purified via silica gel chromatography (30%->100% ethyl acetate/hexanes) to afford 1.025 g of the boc-protected intermediate (beige solids, LRMS: [M+H]+ found 701.90). To a suspension of boc-protected intermediate in dichloromethane (5 mL), TFA (1 mL) was added. The reaction mixture was stirred at room temperature for 16 hours. The reaction mixture was concentrated in vacuo to afford the title compound (1.563 g, 73% yield over 2 steps). LRMS: [M+H]+ found 601.81)

### 2-(((2-((4-(4-(2-(2-(2-((2-(2,6-Dioxopiperidin-3-yl)-1,3-dioxoisoindolin-4-yl)amino)ethoxy)ethoxy)ethyl)piperazin-1-yl)-3-(methylsulfonyl)phenyl)amino)-5-fluoropyrimidin-4-yl)(5-(hydroxymethyl)-2-methylphenyl)amino)methyl)benzonitrile

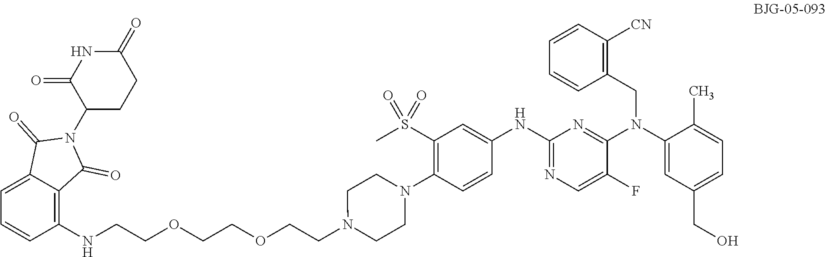 [SMILES: Cc1ccc(CO)cc1N(Cc1ccccc1C#N)c1nc(Nc2ccc(N3CCN(CCOCCOCCNc4cccc5c4C(=O)N(C4CCC(=O)NC4=O)C5=O)CC3)c(S(C)(=O)=O)c2)ncc1F]

To a solution of solution of 2-(2,6-dioxopiperidin-3-yl)-4-((2-(2-(2-hydroxyethoxy)ethoxy)ethyl)amino)isoindoline-1,3-dione (20.3 mg, 0.050 mmol, 1 equiv) in dichloromethane (1.0 mL), dess-martin periodane (31.8 mg, 0.075 mmol, 1.5 equiv) was added. After 16 hours of stirring at room temperature, the reaction mixture was diluted with 5.0 mL of dichloromethane, filtered and concentrated. To a solution of the crude aldehyde and 2-(((5-fluoro-2-((3-(methylsulfonyl)-4-(piperazin-1-yl)phenyl)amino)pyrimidin-4-yl)(5-(hydroxymethyl)-2-methylphenyl)amino)methyl) benzonitrile (24.9 mg, 0.030 mmol, 1 equiv) in methanol (0.60 mL), sodium cyanoborohydride (5.6 mg, 0.090 mmol, 3 equiv) was added. After 16 hours of stirring at room temperature, the reaction mixture was diluted with 2.0 mL of methanol, filtered and purified by reverse-phase prep HPLC (95-15% H₂O/MeOH, 40 mL/min, 45 min). Lyophilization from H₂O/MeCN provided the title compound as a yellow powder (8.8 mg, 18% yield TFA salt). ¹H NMR (500 MHz, DMSO-d₆) δ 11.09 (s, 1H), 9.65 (s, 1H), 8.29 (s, 1H), 8.02 (d, J=5.6 Hz, 1H), 7.78 (d, J=7.7 Hz, 2H), 7.71-7.60 (m, 2H), 7.58 (dd, J=8.6, 7.1 Hz, 1H), 7.45 (td, J=7.5, 1.5 Hz, 1H), 7.31 (d, J=8.8 Hz, 1H), 7.23-7.12 (m, 3H), 7.06-6.99 (m, 2H), 6.61 (t, J=5.8 Hz, 1H), 5.52 (s, 1H), 5.12 (t, J=5.6 Hz, 1H), 5.05 (dd, J=12.8, 5.4 Hz, 1H), 4.37 (d, J=5.5 Hz, 2H), 3.67-3.44 (m, 11H), 3.30 (s, 3H), 2.94-2.80 (m, 3H), 2.62-2.51 (m, 3H), 2.03 (s, 3H), 2.01-1.98 (m, 1H). LRMS (ESI) calculated for [M+H]⁺ 989.37. found 988.73.

### 2-(((2-((4-(4-(3-(2-(2-((2-(2,6-Dioxopiperidin-3-yl)-1,3-dioxoisoindolin-4-yl)amino)ethoxy)ethoxy)propanoyl)piperazin-1-yl)-3-(methylsulfonyl)phenyl)amino)-5-fluoropyrimidin-4-yl)(5-(hydroxymethyl)-2-methylphenyl)amino)methyl)benzonitrile

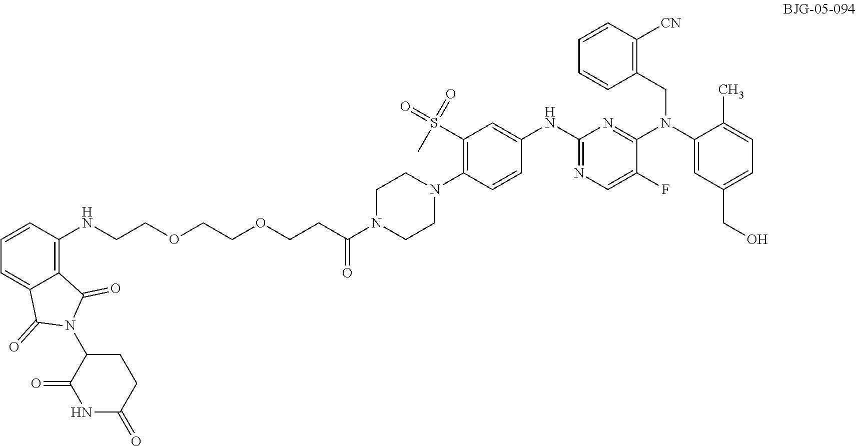 [SMILES: Cc1ccc(CO)cc1N(Cc1ccccc1C#N)c1nc(Nc2ccc(N3CCN(C(=O)CCOCCOCCNc4cccc5c4C(=O)N(C4CCC(=O)NC4=O)C5=O)CC3)c(S(C)(=O)=O)c2)ncc1F]

To a solution of solution of 3-(2-(2-((2-(2,6-dioxopiperidin-3-yl)-1,3-dioxoisoindolin-4-yl)amino)ethoxy)ethoxy)propanoic acid (20.7 mg, 0.040 mmol, 1 equiv) in N,N-dimethylformamide (0.5 mL), diisopropylamine (34.8 AL, 0.200 mmol, 5 equiv) and HATU (15.2 mg, 0.040 mmol, 1 equiv) were added. After stirring the mixture at room temperature for 5 minutes, 2-(((5-fluoro-2-((3-(methylsulfonyl)-4-(piperazin-1-yl)phenyl)amino)pyrimidin-4-yl)(5-(hydroxymethyl)-2-methylphenyl)amino)methyl) benzonitrile (33.2 mg, 0.040 mmol, 1 equiv) was added. After 2 hours of stirring, the reaction mixture was diluted with 1.0 mL of N,N-dimethylformamide and purified by reverse-phase prep HPLC (95-15% H₂O/MeOH, 40 mL/min, 45 min). Lyophilization from H₂O/ACN provided the title compound as a yellow powder (11.7 mg, 29% yield TFA salt). ¹H NMR (500 MHz, DMSO-d₆) δ 11.09 (s, 1H), 9.66 (s, 1H), 8.31 (d, J=2.6 Hz, 1H), 8.02 (d, J=5.6 Hz, 1H), 7.78 (dd, J=7.8, 1.3 Hz, 2H), 7.70-7.61 (m, 2H), 7.57 (dd, J=8.6, 7.0 Hz, 1H), 7.45 (td, J=7.5, 1.4 Hz, 1H), 7.30 (d, J=8.7 Hz, 1H), 7.23-7.10 (m, 3H), 7.06-6.97 (m, 2H), 6.61 (t, J=5.8 Hz, 1H), 5.52 (s, 1H), 5.11 (t, J=5.7 Hz, 1H), 5.05 (dd, J=12.7, 5.4 Hz, 1H), 4.36 (d, J=5.6 Hz, 2H), 3.69-3.60 (m, 4H), 3.60-3.50 (m, 5H), 3.46 (q, J=5.6 Hz, 2H), 2.94-2.77 (m, 5H), 2.63-2.51 (m, 4H), 2.06-1.97 (m, 4H). LRMS (ESI) calculated for [M+H]⁺ 1017.36. found 1016.63.

### 2-(((2-((4-(4-(14-((2-(2,6-Dioxopiperidin-3-yl)-1,3-dioxoisoindolin-4-yl)amino)-3,6,9,12-tetraoxatetradecyl)piperazin-1-yl)-3-(methylsulfonyl)phenyl)amino)-5-fluoropyrimidin-4-yl)(5-(hydroxymethyl)-2-methylphenyl)amino)methyl)benzonitrile

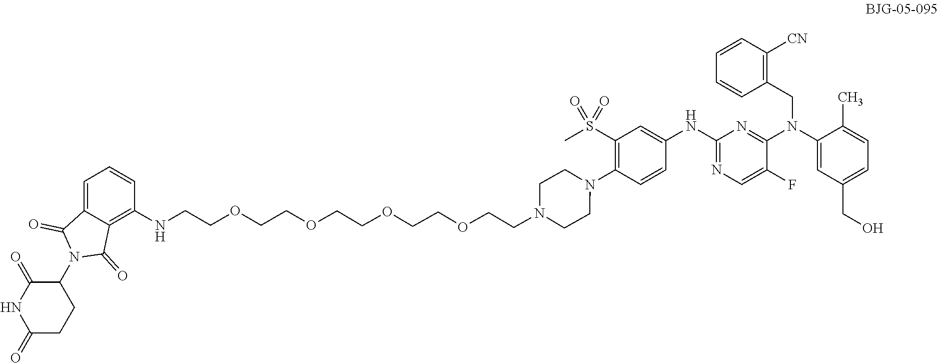 [SMILES: Cc1ccc(CO)cc1N(Cc1ccccc1C#N)c1nc(Nc2ccc(N3CCN(CCOCCOCCOCCOCCNc4cccc5c4C(=O)N(C4CCC(=O)NC4=O)C5=O)CC3)c(S(C)(=O)=O)c2)ncc1F]

To a solution of solution of 2-(2,6-dioxopiperidin-3-yl)-4-((14-hydroxy-3,6,9,12-tetraoxatetradecyl)amino)isoindoline-1,3-dione (39.5 mg, 0.080 mmol, 1 equiv) in dichloromethane (1.6 mL), dess-martin periodane (50.9 mg, 0.120 mmol, 1.5 equiv) was added. After 16 hours of stirring at room temperature, the reaction mixture was diluted with 5.0 mL of dichloromethane, filtered and concentrated. To a solution of the crude aldehyde and 2-(((5-fluoro-2-((3-(methylsulfonyl)-4-(piperazin-1-yl)phenyl)amino) pyrimidin-4-yl)(5-(hydroxymethyl)-2-methylphenyl)amino)methyl)benzonitrile (66.4 mg, 0.080 mmol, 1 equiv) in methanol (1.0 mL), sodium cyanoborohydride (15.1 mg, 0.240 mmol, 3 equiv) was added. After 16 hours of stirring at room temperature, the reaction mixture was diluted with 2.0 mL of methanol, filtered and purified by reverse-phase prep HPLC (95-15% H2O/MeOH, 40 mL/min, 45 min). Lyophilization from H₂O/MeCN provided the title compound as a yellow powder (9.6 mg, 11% yield TFA salt). ¹H NMR (500 MHz, DMSO-d6) δ 11.09 (s, 1H), 9.63 (s, 1H), 8.27 (d, J=2.6 Hz, 1H), 8.01 (d, J=5.6 Hz, 1H), 7.95 (dd, J=7.9, 1.2 Hz, 1H), 7.81-7.71 (m, 2H), 7.70-7.60 (m, 3H), 7.57 (dd, J=8.6, 7.0 Hz, 1H), 7.45 (td, J=7.5, 1.3 Hz, 2H), 7.33 (d, J=8.8 Hz, 1H), 7.23-7.15 (m, 3H), 7.13 (d, J=8.6 Hz, 1H), 7.06-6.95 (m, 2H), 6.60 (t, J=5.8 Hz, 1H), 5.51 (s, 1H), 5.27-4.92 (m, 3H), 4.37 (s, 2H), 3.61 (t, J=5.4 Hz, 2H), 3.59-3.42 (m, 20H), 2.95-2.81 (m, 5H), 2.62-2.55 (m, 1H), 2.07-1.97 (br s, 4H). LRMS (ESI) calculated for [M+H]⁺ 1077.42. found 1076.65.

### Example 3: Protocols

### Cell Lines

MCF7 (female, CVCL_0031) were cultured in high-glucose DMEM medium (Gibco) supplemented with 10% fetal bovine serum (HyClone), 2 mM L-glutamine and 100 U/ml penicillin/streptomycin (Gibco). MOLT4 (male, CVCL_0013) were cultured in RPMI 1640 medium (Gibco) supplemented with 10% fetal bovine serum and 2 mM L-glutamine. OVCAR3 (female, CVCL_0465), Hey A8 (female, CVCL_8878) and OMM1 (male, CVCL_6939) were cultured in RPMI 1640 medium (Gibco) supplemented with 10% fetal bovine serum, 2 mM L-glutamine and 100 U/ml penicillin/streptomycin (Gibco). All cell lines were cultured at 37° C. in a humidified 5% CO₂ incubator. HEK293 cells stably expressing Nluc-PAK1 were constructed by transfecting with the pFN31K Nluc-PAK1 expression vector (0.5 μg DNA per well) in 12-well plates using Lipofectamine™ 3000 (Invitrogen) according to the manufacturer's protocol. Transfected Nluc-PAK1 cells were cultured for 1 week in DMEM medium containing G418 (2 mg mL⁻¹) to select stable clones.

### Virus

A doxycycline (Dox)-inducible shRNA-bearing retrovirus against PAK1 has been described (Ong, et al. (2011) Proc. Natl. Acad. Sci., 108:7177-7182). PAK1 shRNA-1: 5′-GAT CCCCGAAGAGAGGTTCAGCTAAATTCAAGAGATTTAGCTGAACCTCTCTTCTTTTTT GGAAA-3′ (SEQ ID NO: 1). Recombinant viruses were generated using the Phoenix amphotropic packaging system (Orbigen). The ONX cells were transfected using Lipofectamine™ 3000 (Invitrogen). Viral supernatants were harvested 72 hours post-transfection and filtered. Ovarian (OVCAR3) and breast (MCF7) cancer cells were incubated with retroviral supernatant supplemented with 8 μg/mL polybrene for 4 hours at 37° C., and then were cultured in growth media for 48 hours for viral integration. Green fluorescent protein (GFP)-positive infected cells were selected by flow cytometry.

### Plasmids

The pFN31K-Nluc-PAK1 vector was constructed as follows: The gene sequence encoding PAK1 from pCMV6M-PAK1 (Plasmid #12209 Addgene) was PCR-amplified using the following oligonucleotide pair:

(Forward)
(SEQ ID NO: 2)
TTCTGGCGGGCTCGAGCGTCGACATGGAACAGAAACT,
(Reverse)
(SEQ ID NO: 3)
TACCGAGCCCGAATTGAATTCCTCGAGGCCACGAAG,

designed with recognition sites for XhoI and EcoRI restriction enzymes. The PCR product was subcloned into the expression vector pFN31K-Nluc) using the XhoI and EcoRI restriction endonucleases and in-Fusion HD Enzyme (Takara, Japan).

### Retroviral Transductions

The ΦNX packaging cell line (Orbigen) was transfected using Lipofectamine™ 2000 according to the manufacturer's instruction. Viral supernatants were harvested 48 hours post-transfection and filtered. Cells were incubated with retroviral supernatant supplemented with 4 μg/ml polybrene for 4 hours at 37° C., and then were cultured in growth media for 48 hours for viral integration. Green fluorescent protein (GFP)-positive infected cells were selected by flow cytometry.

Drug Treatment with PAK1 Degraders

NVS-PAK1-1, BJG-05-039, BJG-05-098, bortezomib (Selleckchem), valspodar (Sigma-Aldrich), and lenalidomide (Selleckchem) were dissolved in DMSO at 10 mM. Cells were seeded in 6-well plate at 250,000 cells per mL in 2 mL per well. Cells were incubated overnight then treated with various concentrations of Pak degraders alone or together with bortezomib or lenalidomide for 24 hours. Protein lysates were harvested at the times specified.

### Immunoblotting

Cells were washed once in PBS and lysed in RIPA buffer (50 mM Tris-HCl, 150 mM NaCl, 0.25% (w/v) sodium deoxycholate, 1% (w/v) NP-40, pH 7.5) containing protease inhibitor cocktail (Roche) and phosphatase inhibitor cocktail (Roche) for 30 minutes on ice. Cell lysates were obtained by centrifugation at 13,500 rpm for 15 minutes at 4° C. BCA protein assay (Thermo Scientific) was used to determine protein concentration then equal amounts of total proteins were separated by SDS-PAGE (Bio-Rad) and transferred to PVDF membrane (Thermo Scientific) at 100V for 2 hours. Membranes were blocked in 5% (w/v) non-fat dry milk in tris-buffered saline with 0.1% Tween-20 (TBS-T) for 1 hour and incubated with primary antibody at 4° C. for overnight. Membranes were washed with TBS-T on the next day and incubated with HRP-conjugated secondary antibodies (Millipore) at room temperature for 1 hour and exposed to films after washing.

### Cell Viability Assay

Cells were plated at 2×10³ cells per well in 96-well plates overnight and treated with various concentrations of Pak degraders for 72 hours. Cell viability was measured by MTT assay and the half maximal inhibitory concentration (IC₅₀) was calculated using GraphPad Prism. Triplicates were performed for each sample and medium alone was used as a blank.

### Luciferase Assays

HEK293 cells stably expressing pFN31K-Nluc-PAK1 were assayed for luciferase activity according to the manufacturer's Nano-Glo® Live Cell Assay System protocol (Promega). In brief, 25 μl of Nano-Glo® Live Cell Reagent was added per well and the plate was gently mixed by hand, then placed in a 37° C. luminometer for 10 minutes.

### KinomeScan®

The kinase engagement assay (KINOMEscan®) was performed by DiscoverX assessing binding abilities toward a set of kinases. NVS-PAK1-1 was screened at a concentration of 1 μM and BJG-05-039 was screened at a concentration of 10 μM.

### Kinase Activity Assay

Kinase activity assays were performed by Reaction Biology Corp. Compounds were tested in 10-dose IC₅₀ duplicate mode with a 3-fold serial dilution starting at 1 μM. The control compound, staurosporine, was tested in 10-dose IC₅₀ mode with 4-fold serial dilution starting at 20 μM. Reactions were carried out at 10 μM ATP. IC₅₀ values were calculated using Prism 7.0 (GraphPad).

### Biochemical Assay Protocol for PAK1 and PAK2

The activity/inhibition of human recombinant PAK1 (kinase domain) or PAK2 (full length) was determined by measuring the phosphorylation of a FRET peptide substrate (Ser/Thr19) labeled with Coumarin and Fluorescein using Z′-LYTE™ assay (Invitrogen). The 10 μL assay mixtures contained 50 mM HEPES (pH 7.5), 0.01% Brij-35, 10 mM MgCl₂, 1 mM EGTA, 2 μM FRET peptide substrate, and PAK enzyme (20 pM PAK1; 50 pM PAK2). Incubations were carried out at 22° C. in black polypropylene 384-well plates (Corning Costar). Prior to the assay, enzyme, FRET peptide substrate and serially diluted test compounds were preincubated together in assay buffer (7.5 μL) for 10 minutes, and the assay was initiated by the addition of 2.5 μL assay buffer containing 4×ATP (160 μM PAK1; 480 μM PAK2). Following the 60-minute incubation, the assay mixtures were quenched by the addition of 5 μL of Z′-LYTE™ development reagent, and 1 hour later the emissions of Coumarin (445 nm) and Fluorescein (520 nm) were determined after excitation at 400 nm using an Envision plate reader (Perkin Elmer). An emission ratio (445 nm/520 nm) was determined to quantify the degree of substrate phosphorylation.

### TMT LC-MS Sample Preparation

MOLT4 cells were treated with DMSO in biological triplicate and 1 μM BJG-05-039 for 5 hours and harvested by centrifugation. Cell lysis was performed by the addition of Urea buffer (8 M Urea, 50 mM NaCl, 50 mM 4-(2-hydroxyethyl)-1-piperazineethanesulfonic acid (EPPS) pH 8.5, Protease and Phosphatase inhibitors) followed by manual homogenization by 20 passes through a 21-gauge (1.25 in. long) needle. Lysate was clarified by centrifugation at 4° C. and protein quantified using bradford (Bio-Rad) assay. 100 μg of protein for each sample was reduced, alkylated and precipitated using methanol/chloroform as described (Donovan, et al. (2018) Elife 7: e38430). The resulting precipitated protein was resuspended in 4 M Urea, 50 mM HEPES pH 7.4, buffer for solubilization, followed by dilution to 1 M urea with the addition of 200 mM EPPS, pH 8. Proteins were digested for 12 hours at room temperature with LysC (1:50 ratio), followed by dilution to 0.5 M urea and a second digestion step was performed by addition of trypsin (1:50 ratio) for 6 hours at 37° C. Anhydrous ACN was added to each peptide sample to a final concentration of 30%, followed by addition of Tandem mass tag (TMT) reagents at a labelling ratio of 1:4 peptide:TMT label. TMT labelling occurred over a 1.5 hour incubation at room temperature followed by quenching with the addition of hydroxylamine to a final concentration of 0.3%. Each of the samples were combined using adjusted volumes and dried down in a speed vacuum followed by desalting with C18 SPE (Sep-Pak, Waters). The sample was offline fractionated into 96 fractions by high pH reverse-phase HPLC (Agilent LC1260) through an aeris peptide xb-c18 column (phenomenex) with mobile phase A containing 5% acetonitrile and 10 mM NH₄HCO₃ in LC-MS grade H2O, and mobile phase B containing 90% acetonitrile and 5 mM NH₄HCO₃ in LC-MS grade H₂O (both pH 8.0). The resulting 96 fractions were recombined in a non-contiguous manner into 24 fractions and desalted using C18 solid phase extraction plates (SOLA, Thermo Fisher Scientific) followed by subsequent mass spectrometry analysis.

Data were collected using an Orbitrap Fusion Lumos mass spectrometer (Thermo Fisher Scientific, San Jose, CA) coupled with an Proxeon EASY-nLC 1200 LC lump (Thermo Fisher Scientific). Peptides were separated on a 50 cm 75 μm inner diameter EasySpray ES903 microcapillary column (Thermo Fisher Scientific). Peptides were separated over a 190 min gradient of 6-27% acetonitrile in 1.0% formic acid with a flow rate of 300 nL/min.

Quantification was performed using a MS3-based TMT method as described (Donovan et al. (2020) Cell 183(6):1714-1731). The data were acquired using a mass range of m/z 340-1350, resolution 120,000, AGC target 5×10⁵, maximum injection time 100 ms, dynamic exclusion of 120 seconds for the peptide measurements in the Orbitrap. Data dependent MS2 spectra were acquired in the ion trap with a normalized collision energy (NCE) set at 35%, AGC target set to 1.8×10⁴ and a maximum injection time of 120 ms. MS3 scans were acquired in the Orbitrap with HCD collision energy set to 55%, AGC target set to 2×10⁵, maximum injection time of 150 ms, resolution at 50,000 and with a maximum synchronous precursor selection (SPS) precursors set to 10.

### LC-MS Data Analysis

Proteome Discoverer 2.4 (Thermo Fisher Scientific) was used for .RAW file processing and controlling peptide and protein level false discovery rates, assembling proteins from peptides, and protein quantification from peptides. The MS/MS spectra were searched against a Swissprot human database (December 2019) containing both the forward and reverse sequences. Searches were performed using a 20 ppm precursor mass tolerance, 0.6 Da fragment ion mass tolerance, tryptic peptides containing a maximum of two missed cleavages, static alkylation of cysteine (57.02146 Da), static TMT labelling of lysine residues and N-termini of peptides (304.2071 Da), and variable oxidation of methionine (15.99491 Da). TMT reporter ion intensities were measured using a 0.003 Da window around the theoretical m/z for each reporter ion in the MS3 scan. The peptide spectral matches with poor quality MS3 spectra were excluded from quantitation (summed signal-to-noise across channels <100 and precursor isolation specificity <0.5), and the resulting data was filtered to only include proteins with a minimum of 2 unique peptides quantified. Reporter ion intensities were normalized and scaled using in-house scripts in the R framework. Statistical analysis was carried out using the limma package within the R framework (Ritchie, et al. (2015) Nucleic Acids Res., 43:e47).

### Statistics

All experiments were performed at least three times. Results were reported as means±SD. The significance of the data was determined by two-tailed, unpaired Student's t-test with p<0.05 considered statistically significant.

### Example 4: Results

### Design of a Selective PAK1 Degrader

To develop a PAK1-selective degrader, compounds based on NVS-PAK1-1—a unique allosteric inhibitor which displays marked selectivity for PAK1 over PAK2—were designed. NVS-PAK1-1 is based on a dibenzodiazepine scaffold, which is uncommon for kinase inhibitors. Co-crystals of close relatives of NVS-PAK1-1 show that these molecules bind beneath the αC helix in PAK1 in a pocket formed in the DFG-out conformation of PAK1 analogous to the well-characterized allosteric inhibitors of MEK1/2 (Alessi, et al. (1995) J. Biol. Chem., 270:27489-27494) and EGFR (Jia, et al. (2016) Nature 534:129-132). It has been speculated that they derive specificity for PAK1 over PAK2 due to a steric clash between the molecule with Leu301 in PAK2 vs. the equivalent Asn322 in PAK1 (Karpov, et al. (2015) ACS Med. Chem. Lett., 6:776-781). Such specificity is unusual and has rarely been described for other inhibitors of Group A PAK. For example, NVS-PAK1-1 and over 2000 hinge-region kinase inhibitors against PAK1 and PAK2 were tested. As seen in FIG. 1A, NVS-PAK1-1 displayed the greatest differential sensitivities between these two kinases.

As NVS-PAK1-1 has a short in vivo half-life and has shown marginal effects in cancer cell lines (Karpov, et al. (2015) ACS Med. Chem. Lett., 6:776-781), degrader forms of this compound were designed. Such compounds could have the added advantage over conventional PAK inhibitors because, in addition to blocking kinase enzymatic activity, they also have the potential to reduce signaling effects that emanate from the scaffold functions of PAK1. A co-crystal structure of a close analogue of NVS-PAK1-1 bound to PAK1 (PDB: 4ZJJ) revealed that the isopropyl urea is solvent exposed, indicating that the carbonyl (either as a urea or an amide) could serve as a suitable attachment site for linkers without adversely affecting affinity to PAK1.

Hydrocarbon and polyethylene glycol (PEG) linkers of varying lengths were used to conjugate NVS-PAK1-1 with either a CRBN ligand (thalidomide/pomalidomide) or a VHL ligand, respectively. To verify that conjugation of the linker and thalidomide/pomalidomide or VHL ligand did not affect the ability of NVS-PAK1-1 to bind to PAK1, different NVS-PAK1-1 conjugates were tested in a commercially available fluorescence resonance energy transfer-based assay (Invitrogen, Z′-Lyte) for PAK1, PAK2, and PAK4 inhibition (Table 1). Three of these—BJG-05-014, BJG-05-027, and BJG-05-039—retained PAK1 inhibitory activity and selectivity and were therefore selected for more detailed studies. While BJG-05-039 (FIG. 1B) had lower inhibitory activity against PAK1 (half maximal inhibitory concentration (IC₅₀)=233 nM) compared to NVS-PAK1-1 (IC₅₀=29.6 nM), it displayed greater specificity (PAK2 IC₅₀>10,000 nM for BJG-05-039 vs. 824 nM for NVS-PAK1-1), demonstrating that BJG-05-039 had surprisingly improved selectivity for PAK1 over PAK2 compared to the parent molecule. Molecular modeling indicates that the addition of the linker and degrader moieties would not impair selective binding of the inhibitory ligand in the ATP cleft of PAK1.

TABLE 1
Structure and properties of PAK1 degraders.

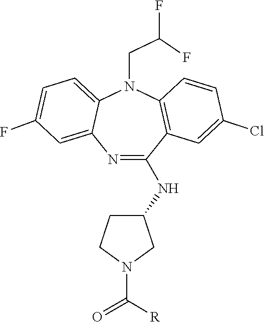

| | | PAK1 | PAK2 | PAK4 |
|---|---|---|---|---|
| | | IC₅₀ | IC₅₀ | IC₅₀ |
| Cpd. | R | (nM) | (nM) | (nM) |
| NVS- PAK- 1 | 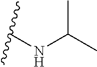 [SMILES: *NC(C)C] | 29.6 | 824 | >10000 |
| BJG- 05- 014 | 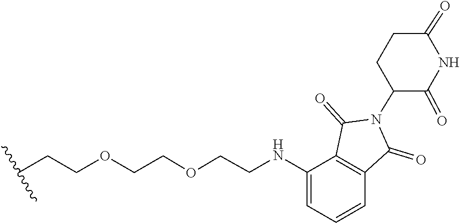 [SMILES: *CCOCCOCCNc1cccc2c1C(=O)N(C1CCC(=O)NC1=O)C2=O] | 432 | 724 | 152 |
| BJG- 05- 022 | 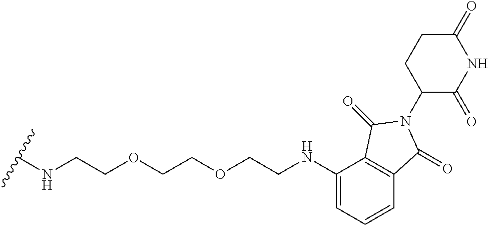 [SMILES: *NCCOCCOCCNc1cccc2c1C(=O)N(C1CCC(=O)NC1=O)C2=O] | 1490 | 6750 | 3860 |
| BJG- 05- 019 | 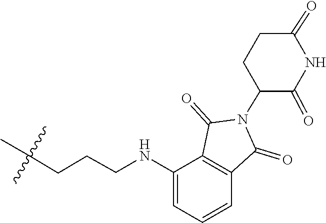 [SMILES: *CCCNc1cccc2c1C(=O)N(C1CCC(=O)NC1=O)C2=O] | 6300 | >10000 | >10000 |
| BJG- 05- 017 | 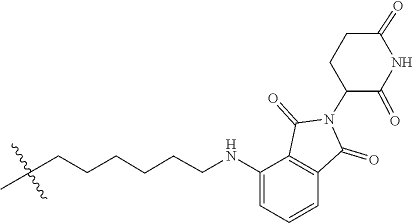 [SMILES: *CCCCCCNc1cccc2c1C(=O)N(C1CCC(=O)NC1=O)C2=O] | >1110 | >3330 | >1000 |
| BJG- 05- 018 | 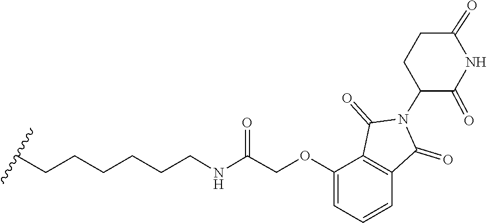 [SMILES: *CCCCCCNC(=O)COc1cccc2c1C(=O)N(C1CCC(=O)NC1=O)C2=O] | >3330 | >10000 | >10000 |
| BJG- 05- 027 | 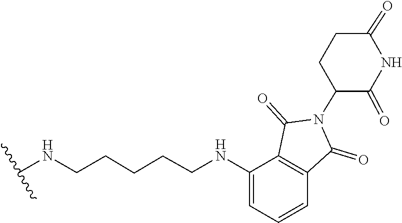 [SMILES: *NCCCCCNc1cccc2c1C(=O)N(C1CCC(=O)NC1=O)C2=O] | 594 | >10000 | >10000 |
| BJG- 05- 039 | 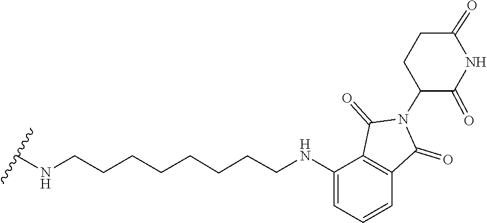 [SMILES: *NCCCCCCCCNc1cccc2c1C(=O)N(C1CCC(=O)NC1=O)C2=O] | 233 | >10000 | >10000 |
| BJG- 05- 020 | 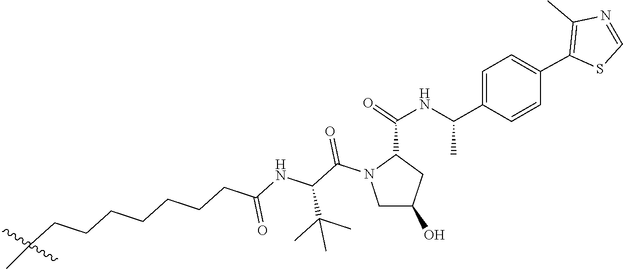 | >10000 | >10000 | >10000 |
| BJG- 05- 052 | 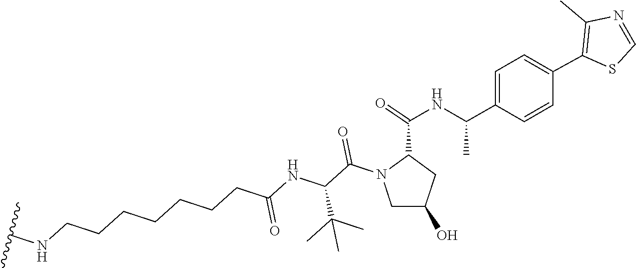 | n/a | n/a | n/a |
| BJG- 05- 053 | 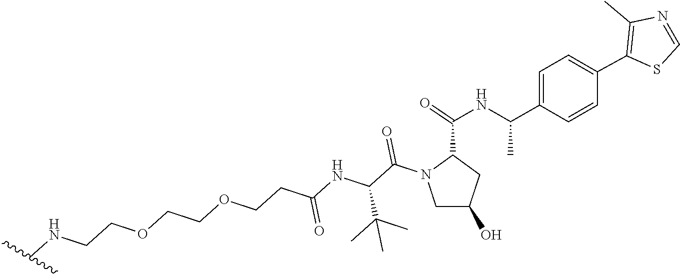 | n/a | n/a | n/a |
| BJG- 05- 098 | 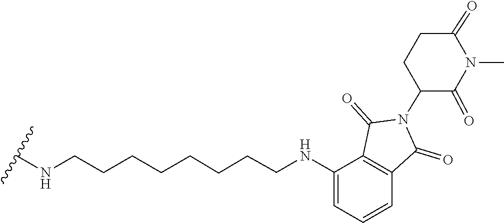 [SMILES: *NCCCCCCCCNc1cccc2c1C(=O)N(C1CCC(=O)N(C)C1=O)C2=O] | n/a | n/a | n/a |
| BJG- 05- 026 | 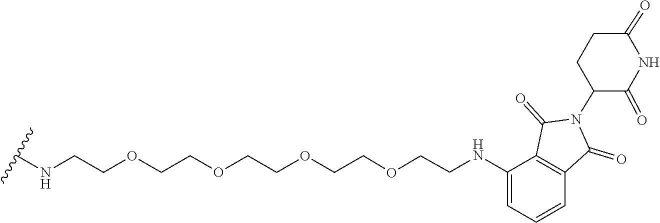 [SMILES: *NCCOCCOCCOCCOCCNc1cccc2c1C(=O)N(C1CCC(=O)NC1=O)C2=O] | n/a | n/a | n/a |

BJG-05-014, BJG-05-027, and BJG-05-039 were tested in Panc1 cells for their ability to degrade PAK1. Of these three molecules, BJG-05-039, which uses an 8-carbon linker to conjugate NVS-PAK1-1 with pomalidomide, had the most promising profile (FIG. 1C and FIG. 1D).

TABLE 2
Structure and properties of ATP-competitive PAK1 degraders.

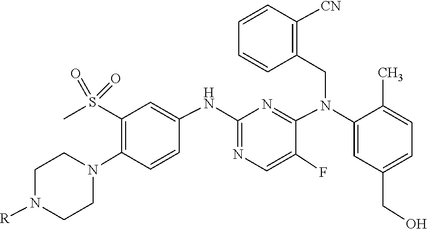

| | | PAK1 | PAK2 | PAK4 |
|---|---|---|---|---|
| | | IC₅₀ | IC₅₀ | IC₅₀ |
| Cpd. | R | (nM) | (nM) | (nM) |
| BJG- 05- 093 | 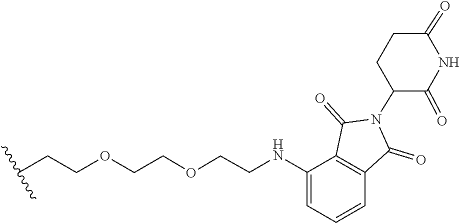 [SMILES: *CCOCCOCCNc1cccc2c1C(=O)N(C1CCC(=O)NC1=O)C2=O] | 32.5 | n/a | n/a |
| BJG- 05- 094 | 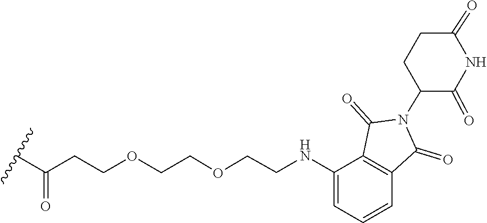 [SMILES: *C(=O)CCOCCOCCNc1cccc2c1C(=O)N(C1CCC(=O)NC1=O)C2=O] | 103 | n/a | n/a |
| BJG- 05- 095 | 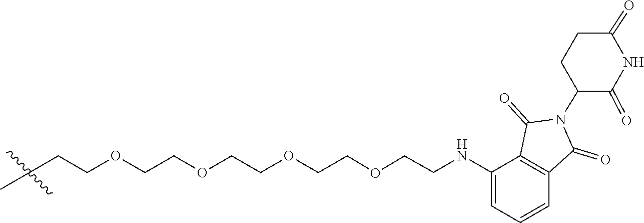 [SMILES: *CCOCCOCCOCCOCCNc1cccc2c1C(=O)N(C1CCC(=O)NC1=O)C2=O] | 36.6 | n/a | n/a |

The biochemical selectivity of BJG-05-039 was also evaluated against a panel of 468 kinases at 10 μM (KINOMEscan®). These tests revealed that BJG-05-039 had a similar selectivity profile as 1 μM NVS-PAK1-1 (Table 3).

TABLE 3
KINOMEscan ® Profiling of BJG-05-039 @ 10 μM.
BJG-05-039 @ 10 μM

| DiscoveRx Gene | Entrez Gene | Percent |
|---|---|---|
| Symbol | Symbol | Control |
| PAK1 | PAK1 | 20 |
| PRKD3 | PRKD3 | 37 |
| MARK2 | MARK2 | 38 |
| CAMK2A | CAMK2A | 39 |
| DAPK3 | DAPK3 | 40 |
| PHKG2 | PHKG2 | 40 |
| DAPK2 | DAPK2 | 41 |
| JAK1(JH1domain-catalytic) | JAK1 | 42 |
| PAK7 | PAK7 | 42 |
| RPS6KA5(Kin.Dom.2-C-terminal) | RPS6KA5 | 42 |
| WEE1 | WEE1 | 42 |
| ARK5 | NUAK1 | 45 |
| CAMK2D | CAMK2D | 45 |
| MAP4K4 | MAP4K4 | 45 |
| NDR2 | STK38L | 45 |
| RSK4(Kin.Dom.2-C-terminal) | RPS6KA6 | 45 |
| ACVR2B | ACVR2B | 46 |
| CAMK2B | CAMK2B | 46 |
| CAMKK2 | CAMKK2 | 46 |
| RET | RET | 46 |
| TLK1 | TLK1 | 46 |
| DAPK1 | DAPK1 | 47 |
| EPHA3 | EPHA3 | 47 |
| AAK1 | AAK1 | 48 |
| PRKX | PRKX | 48 |
| RSK1(Kin.Dom.2-C-terminal) | RPS6KA1 | 48 |
| TNIK | TNIK | 48 |
| AXL | AXL | 49 |
| GSK3A | GSK3A | 50 |
| ACVR2A | ACVR2A | 51 |
| BRSK1 | BRSK1 | 51 |
| CAMK2G | CAMK2G | 51 |
| RSK3(Kin.Dom.2-C-terminal) | RPS6KA2 | 51 |
| CAMK1 | CAMK1 | 52 |
| CAMKK1 | CAMKK1 | 52 |
| CSNK1G1 | CSNK1G1 | 52 |
| DDR1 | DDR1 | 52 |
| DMPK | DMPK | 52 |
| ERK4 | MAPK4 | 52 |
| MST3 | STK24 | 52 |
| NEK2 | NEK2 | 52 |
| CDC2L1 | CDK11B | 53 |
| CDK9 | CDK9 | 53 |
| FLT3 | FLT3 | 53 |
| NLK | NLK | 53 |
| PRKD2 | PRKD2 | 53 |
| PKMYT1 | PKMYT1 | 54 |
| IRAK3 | IRAK3 | 55 |
| TTK | TTK | 55 |
| GAK | GAK | 56 |
| GCN2(Kin.Dom.2, S808G) | EIF2AK4 | 56 |
| AKT3 | AKT3 | 57 |
| CHEK1 | CHEK1 | 57 |
| CLK1 | CLK1 | 57 |
| ERK5 | MAPK7 | 57 |
| PRKG1 | PRKG1 | 57 |
| DRAK2 | STK17B | 58 |
| PCTK2 | CDK17 | 58 |
| DRAK1 | STK17A | 59 |
| ERBB3 | ERBB3 | 59 |
| FLT3(K663Q) | FLT3 | 59 |
| MAP4K5 | MAP4K5 | 59 |
| RET(V804M) | RET | 59 |
| RPS6KA4(Kin.Dom.1-N-terminal) | RPS6KA4 | 59 |
| CSNK1D | CSNK1D | 60 |
| MAPKAPK2 | MAPKAPK2 | 60 |
| MUSK | MUSK | 60 |
| p38-alpha | MAPK14 | 60 |
| PIK3CA | PIK3CA | 60 |
| CSNK1E | CSNK1E | 61 |
| MET(M1250T) | MET | 61 |
| CHEK2 | CHEK2 | 62 |
| CLK3 | CLK3 | 62 |
| PKAC-beta | PRKACB | 62 |
| STK16 | STK16 | 62 |
| TIE1 | TIE1 | 62 |
| ADCK4 | ADCK4 | 63 |
| CAMK1G | CAMK1G | 63 |
| CSNK1G3 | CSNK1G3 | 63 |
| ERK8 | MAPK15 | 63 |
| FLT3(D835Y) | FLT3 | 63 |
| KIT(V559D, V654A) | KIT | 63 |
| MET(Y1235D) | MET | 63 |
| MRCKA | CDC42BPA | 63 |
| PIK3CA(H1047L) | PIK3CA | 63 |
| ERK3 | MAPK6 | 64 |
| FAK | PTK2 | 65 |
| INSRR | INSRR | 65 |
| NEK5 | NEK5 | 65 |
| PHKG1 | PHKG1 | 65 |
| PKN2 | PKN2 | 65 |
| RIPK1 | RIPK1 | 65 |
| MLCK | MYLK3 | 66 |
| EGFR(S752-1759del) | EGFR | 67 |
| FRK | FRK | 67 |
| HIPK4 | HIPK4 | 67 |
| MRCKB | CDC42BPB | 67 |
| MYO3A | MYO3A | 67 |
| RIPK2 | RIPK2 | 67 |
| TRKB | NTRK2 | 67 |
| CAMK1D | CAMK1D | 68 |
| FGFR4 | FGFR4 | 68 |
| MET | MET | 68 |
| CDK2 | CDK2 | 69 |
| IGF1R | IGF1R | 70 |
| STK39 | STK39 | 70 |
| ACVRL1 | ACVRL1 | 71 |
| FYN | FYN | 71 |
| GRK4 | GRK4 | 71 |
| MAST1 | MAST1 | 71 |
| BRK | PTK6 | 72 |
| DMPK2 | CDC42BPG | 72 |
| LIMK2 | LIMK2 | 72 |
| MAP4K3 | MAP4K3 | 72 |
| PKNB(M. tuberculosis) | pknB | 72 |
| TRKA | NTRK1 | 72 |
| TYRO3 | TYRO3 | 72 |
| YANK3 | STK32C | 72 |
| ASK1 | MAP3K5 | 73 |
| BLK | BLK | 73 |
| BUB1 | BUB1 | 73 |
| TSSK1B | TSSK1B | 73 |
| BIKE | BMP2K | 74 |
| CAMK4 | CAMK4 | 74 |
| CDK3 | CDK3 | 74 |
| CSK | CSK | 74 |
| EGFR | EGFR | 74 |
| EGFR(L858R) | EGFR | 74 |
| EPHB2 | EPHB2 | 74 |
| MTOR | MTOR | 74 |
| WNK1 | WNK1 | 74 |
| PAK4 | PAK4 | 75 |
| PIM1 | PIM1 | 75 |
| STK33 | STK33 | 75 |
| TGFBR2 | TGFBR2 | 75 |
| FLT3(D835V) | FLT3 | 76 |
| MAP3K2 | MAP3K2 | 76 |
| NIK | MAP3K14 | 76 |
| SGK2 | SGK2 | 76 |
| ABL1(F317L)-nonphosphorylated | ABL1 | 77 |
| CDKL2 | CDKL2 | 77 |
| DCAMKL1 | DCLK1 | 77 |
| FGFR2 | FGFR2 | 77 |
| GRK2 | ADRBK1 | 77 |
| PIK3CG | PIK3CG | 77 |
| PRKCQ | PRKCQ | 77 |
| WNK4 | WNK4 | 77 |
| ASK2 | MAP3K6 | 78 |
| CSNK1G2 | CSNK1G2 | 78 |
| CSNK2A2 | CSNK2A2 | 78 |
| EGFR(L747-E749del, A750P) | EGFR | 78 |
| PFTAIRE2 | CDK15 | 78 |
| PIK4CB | PI4KB | 78 |
| PIM3 | PIM3 | 78 |
| QSK | KIAA0999 | 78 |
| ULK1 | ULK1 | 78 |
| WEE2 | WEE2 | 78 |
| DCAMKL3 | DCLK3 | 79 |
| LYN | LYN | 79 |
| NEK7 | NEK7 | 79 |
| PIKFYVE | PIKFYVE | 79 |
| YES | YES1 | 79 |
| CDK8 | CDK8 | 80 |
| EGFR(L747-T751del, Sins) | EGFR | 80 |
| LRRK2 | LRRK2 | 80 |
| MST2 | STK3 | 80 |
| TLK2 | TLK2 | 80 |
| KIT(V559D) | KIT | 81 |
| MELK | MELK | 81 |
| PRKD1 | PRKD1 | 81 |
| PRKR | EIF2AK2 | 81 |
| CDC2L5 | CDK13 | 82 |
| EGFR(L747-S752del, P753S) | EGFR | 82 |
| EPHA2 | EPHA2 | 82 |
| EPHA4 | EPHA4 | 82 |
| FGR | FGR | 82 |
| KIT | KIT | 82 |
| LZK | MAP3K13 | 82 |
| NEK9 | NEK9 | 82 |
| PIM2 | PIM2 | 82 |
| PLK1 | PLK1 | 82 |
| RSK2(Kin.Dom.1-N-terminal) | RPS6KA3 | 82 |
| SBK1 | SBK1 | 82 |
| STK36 | STK36 | 82 |
| TEC | TEC | 82 |
| TNNI3K | TNNI3K | 82 |
| ULK2 | ULK2 | 82 |
| ABL1-nonphosphorylated | ABL1 | 83 |
| ADCK3 | CABC1 | 83 |
| AKT1 | AKT1 | 83 |
| AURKC | AURKC | 83 |
| CSNK1A1L | CSNK1A1L | 83 |
| EGFR(G719C) | EGFR | 83 |
| ERK1 | MAPK3 | 83 |
| FLT3(D835H) | FLT3 | 83 |
| JNK1 | MAPK8 | 83 |
| MARK3 | MARK3 | 83 |
| MLK1 | MAP3K9 | 83 |
| NEK1 | NEK1 | 83 |
| SgK110 | SgK110 | 83 |
| STK35 | STK35 | 83 |
| TNK1 | TNK1 | 83 |
| YSK1 | STK25 | 83 |
| CDC2L2 | CDC2L2 | 84 |
| EGFR(G719S) | EGFR | 84 |
| EPHA8 | EPHA8 | 84 |
| FLT1 | FLT1 | 84 |
| JNK2 | MAPK9 | 84 |
| KIT(L576P) | KIT | 84 |
| LTK | LTK | 84 |
| RIOK1 | RIOK1 | 84 |
| RIOK3 | RIOK3 | 84 |
| ACVR1B | ACVR1B | 85 |
| BMPR1B | BMPR1B | 85 |
| CDK11 | CDK19 | 85 |
| ERK2 | MAPK1 | 85 |
| FGFR3 | FGFR3 | 85 |
| KIT(V559D, T670I) | KIT | 85 |
| MEK4 | MAP2K4 | 85 |
| PLK4 | PLK4 | 85 |
| PYK2 | PTK2B | 85 |
| TAOK3 | TAOK3 | 85 |
| ABL1(T315I)-nonphosphorylated | ABL1 | 86 |
| AKT2 | AKT2 | 86 |
| AMPK-alpha1 | PRKAA1 | 86 |
| CDK4 | CDK4 | 86 |
| DDR2 | DDR2 | 86 |
| EGFR(L858R, T790M) | EGFR | 86 |
| FES | FES | 86 |
| FLT4 | FLT4 | 86 |
| MEK6 | MAP2K6 | 86 |
| MST4 | MST4 | 86 |
| NDR1 | STK38 | 86 |
| PDGFRB | PDGFRB | 86 |
| PDPK1 | PDPK1 | 86 |
| ABL1(Q252H)-nonphosphorylated | ABL1 | 87 |
| ANKK1 | ANKK1 | 87 |
| CAMK1B | PNCK | 87 |
| FLT3(ITD) | FLT3 | 87 |
| KIT(D816V) | KIT | 87 |
| PAK2 | PAK2 | 87 |
| RIPK5 | DSTYK | 87 |
| RSK1(Kin.Dom.1-N-terminal) | RPS6KA1 | 87 |
| RSK2(Kin.Dom.2-C-terminal) | RPS6KA3 | 87 |
| SRPK1 | SRPK1 | 87 |
| EGFR(L861Q) | EGFR | 88 |
| FER | FER | 88 |
| JNK3 | MAPK10 | 88 |
| MLK3 | MAP3K11 | 88 |
| MYO3B | MYO3B | 88 |
| PFTK1 | CDK14 | 88 |
| SGK3 | SGK3 | 88 |
| SRC | SRC | 88 |
| ACVR1 | ACVR1 | 89 |
| CSF1R | CSF1R | 89 |
| CTK | MATK | 89 |
| DYRK2 | DYRK2 | 89 |
| EPHB3 | EPHB3 | 89 |
| GRK1 | GRK1 | 89 |
| LKB1 | STK11 | 89 |
| MAP4K2 | MAP4K2 | 89 |
| NIM1 | MGC42105 | 89 |
| S6K1 | RPS6KB1 | 89 |
| SNRK | SNRK | 89 |
| WNK2 | WNK2 | 89 |
| WNK3 | WNK3 | 89 |
| BMX | BMX | 90 |
| CDK4-cyclinD1 | CDK4 | 90 |
| CDK5 | CDK5 | 90 |
| EPHA7 | EPHA7 | 90 |
| GSK3B | GSK3B | 90 |
| HASPIN | GSG2 | 90 |
| HCK | HCK | 90 |
| MERTK | MERTK | 90 |
| MST1 | STK4 | 90 |
| PIK3C2B | PIK3C2B | 90 |
| PKAC-alpha | PRKACA | 90 |
| RET(M918T) | RET | 90 |
| RSK3(Kin.Dom.1-N-terminal) | RPS6KA2 | 90 |
| TXK | TXK | 90 |
| ABL1(F317I)-nonphosphorylated | ABL1 | 91 |
| EPHB4 | EPHB4 | 91 |
| IKK-alpha | CHUK | 91 |
| ITK | ITK | 91 |
| LCK | LCK | 91 |
| NEK6 | NEK6 | 91 |
| PCTK3 | CDK18 | 91 |
| PIK3CA(E542K) | PIK3CA | 91 |
| SLK | SLK | 91 |
| SRPK2 | SRPK2 | 91 |
| TYK2(JH1domain-catalytic) | TYK2 | 91 |
| DCAMKL2 | DCLK2 | 92 |
| GRK3 | ADRBK2 | 92 |
| HPK1 | MAP4K1 | 92 |
| KIT(A829P) | KIT | 92 |
| p38-delta | MAPK13 | 92 |
| SYK | SYK | 92 |
| TESK1 | TESK1 | 92 |
| TNK2 | TNK2 | 92 |
| ULK3 | ULK3 | 92 |
| AURKB | AURKB | 93 |
| CSNK1A1 | CSNK1A1 | 93 |
| ERN1 | ERN1 | 93 |
| INSR | INSR | 93 |
| MYLK2 | MYLK2 | 93 |
| SRPK3 | SRPK3 | 93 |
| TAOK2 | TAOK2 | 93 |
| YSK4 | MAP3K19 | 93 |
| ABL2 | ABL2 | 94 |
| CIT | CIT | 94 |
| EPHA6 | EPHA6 | 94 |
| FLT3(ITD, F691L) | FLT3 | 94 |
| IKK-beta | IKBKB | 94 |
| LIMK1 | LIMK1 | 94 |
| MEK3 | MAP2K3 | 94 |
| MKNK2 | MKNK2 | 94 |
| PIK3CA(H1047Y) | PIK3CA | 94 |
| PRKCH | PRKCH | 94 |
| ROS1 | ROS1 | 94 |
| RPS6KA5(Kin.Dom.1-N-terminal) | RPS6KA5 | 94 |
| TRPM6 | TRPM6 | 94 |
| VRK2 | VRK2 | 94 |
| CLK2 | CLK2 | 95 |
| FGFR3(G697C) | FGFR3 | 95 |
| FLT3(ITD, D835V) | FLT3 | 95 |
| MAP3K4 | MAP3K4 | 95 |
| PIK3C2G | PIK3C2G | 95 |
| PIP5K1A | PIP5K1A | 95 |
| PKN1 | PKN1 | 95 |
| RSK4(Kin.Dom.1-N-terminal) | RPS6KA6 | 95 |
| SIK | SIK1 | 95 |
| TAOK1 | TAOK1 | 95 |
| CSNK2A1 | CSNK2A1 | 96 |
| LRRK2(G2019S) | LRRK2 | 96 |
| MEK2 | MAP2K2 | 96 |
| PIP5K1C | PIP5K1C | 96 |
| SNARK | NUAK2 | 96 |
| ALK | ALK | 97 |
| EGFR(T790M) | EGFR | 97 |
| EPHB6 | EPHB6 | 97 |
| ERBB4 | ERBB4 | 97 |
| FGFR1 | FGFR1 | 97 |
| JAK3(JH1domain-catalytic) | JAK3 | 97 |
| MLK2 | MAP3K10 | 97 |
| MST1R | MST1R | 97 |
| p38-beta | MAPK11 | 97 |
| PCTK1 | CDK16 | 97 |
| PRKCD | PRKCD | 97 |
| PRP4 | PRPF4B | 97 |
| RAF1 | RAF1 | 97 |
| SIK2 | SIK2 | 97 |
| SRMS | SRMS | 97 |
| TYK2(JH2domain-pseudokinase) | TYK2 | 97 |
| ZAK | ZAK | 97 |
| ABL1(H396P)-nonphosphorylated | ABL1 | 98 |
| BMPR1A | BMPR1A | 98 |
| BRAF | BRAF | 98 |
| CDKL3 | CDKL3 | 98 |
| EIF2AK1 | EIF2AK1 | 98 |
| EPHA1 | EPHA1 | 98 |
| EPHB1 | EPHB1 | 98 |
| LOK | STK10 | 98 |
| MAK | MAK | 98 |
| MARK1 | MARK1 | 98 |
| PFCDPK1(P. falciparum) | CDPK1 | 98 |
| RIPK4 | RIPK4 | 98 |
| TIE2 | TEK | 98 |
| BRSK2 | BRSK2 | 99 |
| CASK | CASK | 99 |
| CDKL1 | CDKL1 | 99 |
| EPHA5 | EPHA5 | 99 |
| ICK | ICK | 99 |
| JAK2(JH1domain-catalytic) | JAK2 | 99 |
| MAP3K15 | MAP3K15 | 99 |
| NEK10 | NEK10 | 99 |
| NEK4 | NEK4 | 99 |
| p38-gamma | MAPK12 | 99 |
| PAK6 | PAK6 | 99 |
| PLK3 | PLK3 | 99 |
| RET(V804L) | RET | 99 |
| RIOK2 | RIOK2 | 99 |
| RPS6KA4(Kin.Dom.2-C-terminal) | RPS6KA4 | 99 |
| YANK2 | STK32B | 99 |
| ABL1(E255K)-phosphorylated | ABL1 | 100 |
| ABL1(F317I)-phosphorylated | ABL1 | 100 |
| ABL1(F317L)-phosphorylated | ABL1 | 100 |
| ABL1(H396P)-phosphorylated | ABL1 | 100 |
| ABL1(M351T)-phosphorylated | ABL1 | 100 |
| ABL1(Q252H)-phosphorylated | ABL1 | 100 |
| ABL1(T315I)-phosphorylated | ABL1 | 100 |
| ABL1(Y253F)-phosphorylated | ABL1 | 100 |
| ABL1-phosphorylated | ABL1 | 100 |
| ALK(C1156Y) | ALK | 100 |
| ALK(L1196M) | ALK | 100 |
| AMPK-alpha2 | PRKAA2 | 100 |
| AURKA | AURKA | 100 |
| BMPR2 | BMPR2 | 100 |
| BRAF(V600E) | BRAF | 100 |
| BTK | BTK | 100 |
| CDK4-cyclinD3 | CDK4 | 100 |
| CDK7 | CDK7 | 100 |
| CDKL5 | CDKL5 | 100 |
| CLK4 | CLK4 | 100 |
| CSF1R-autoinhibited | CSF1R | 100 |
| DLK | MAP3K12 | 100 |
| DYRK1A | DYRK1A | 100 |
| DYRK1B | DYRK1B | 100 |
| EGFR(E746-A750del) | EGFR | 100 |
| ERBB2 | ERBB2 | 100 |
| FLT3(N841I) | FLT3 | 100 |
| FLT3(R834Q) | FLT3 | 100 |
| FLT3-autoinhibited | FLT3 | 100 |
| GRK7 | GRK7 | 100 |
| HIPK1 | HIPK1 | 100 |
| HIPK2 | HIPK2 | 100 |
| HIPK3 | HIPK3 | 100 |
| HUNK | HUNK | 100 |
| IKK-epsilon | IKBKE | 100 |
| IRAK1 | IRAK1 | 100 |
| IRAK4 | IRAK4 | 100 |
| JAK1(JH2domain-pseudokinase) | JAK1 | 100 |
| KIT(D816H) | KIT | 100 |
| KIT-autoinhibited | KIT | 100 |
| LATS1 | LATS1 | 100 |
| LATS2 | LATS2 | 100 |
| MAP3K1 | MAP3K1 | 100 |
| MAP3K3 | MAP3K3 | 100 |
| MAPKAPK5 | MAPKAPK5 | 100 |
| MARK4 | MARK4 | 100 |
| MEK1 | MAP2K1 | 100 |
| MEK5 | MAP2K5 | 100 |
| MINK | MINK1 | 100 |
| MKK7 | MAP2K7 | 100 |
| MKNK1 | MKNK1 | 100 |
| MYLK | MYLK | 100 |
| MYLK4 | MYLK4 | 100 |
| NEK11 | NEK11 | 100 |
| NEK3 | NEK3 | 100 |
| OSR1 | OXSR1 | 100 |
| PAK3 | PAK3 | 100 |
| PDGFRA | PDGFRA | 100 |
| PFPK5(P. falciparum) | MAL13P1.279 | 100 |
| PIK3CA(C420R) | PIK3CA | 100 |
| PIK3CA(E545A) | PIK3CA | 100 |
| PIK3CA(E545K) | PIK3CA | 100 |
| PIK3CA(I800L) | PIK3CA | 100 |
| PIK3CA(M1043I) | PIK3CA | 100 |
| PIK3CA(Q546K) | PIK3CA | 100 |
| PIK3CB | PIK3CB | 100 |
| PIK3CD | PIK3CD | 100 |
| PIP5K2B | PIP4K2B | 100 |
| PIP5K2C | PIP4K2C | 100 |
| PLK2 | PLK2 | 100 |
| PRKCE | PRKCE | 100 |
| PRKCI | PRKCI | 100 |
| PRKG2 | PRKG2 | 100 |
| ROCK1 | ROCK1 | 100 |
| ROCK2 | ROCK2 | 100 |
| SGK | SGK1 | 100 |
| TAK1 | MAP3K7 | 100 |
| TBK1 | TBK1 | 100 |
| TGFBR1 | TGFBR1 | 100 |
| TRKC | NTRK3 | 100 |
| TSSK3 | TSSK3 | 100 |
| VEGFR2 | KDR | 100 |
| VPS34 | PIK3C3 | 100 |
| YANK1 | STK32A | 100 |
| ZAP70 | ZAP70 | 100 |

### BJG-05-039 is a Highly Selective PAK1 Degrader

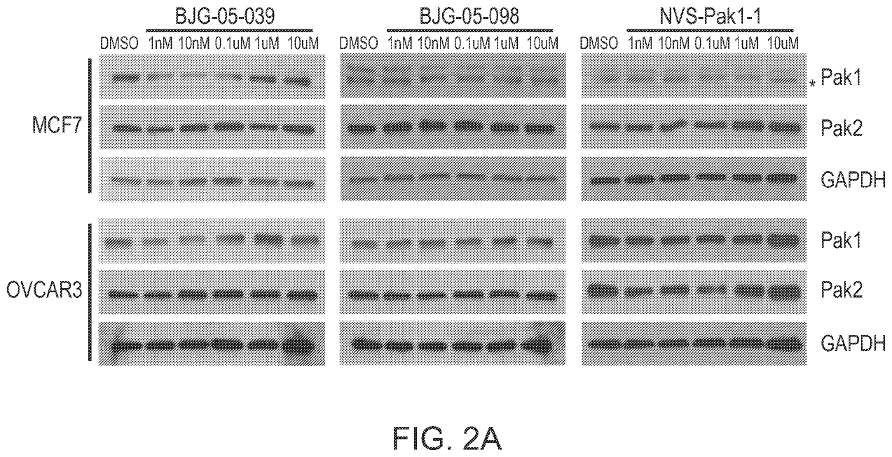

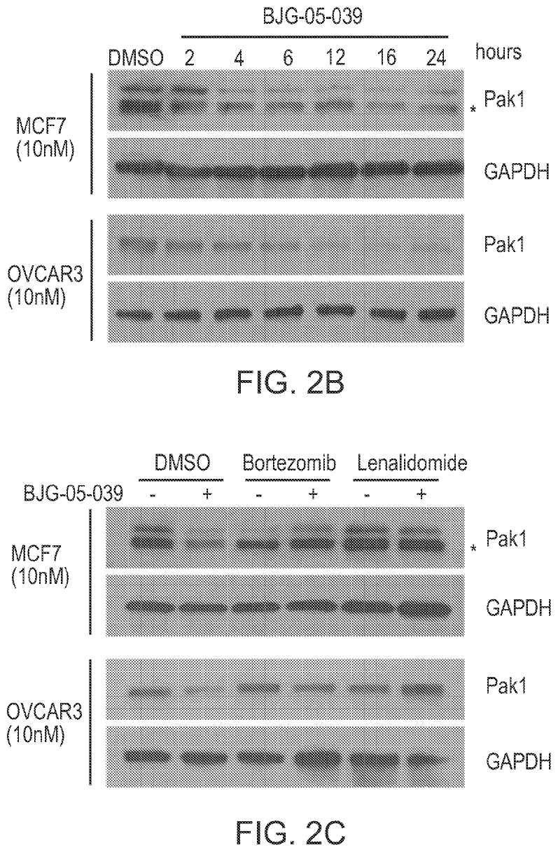

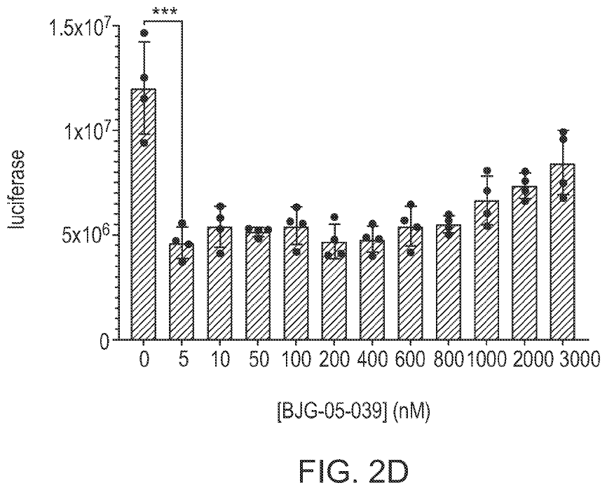

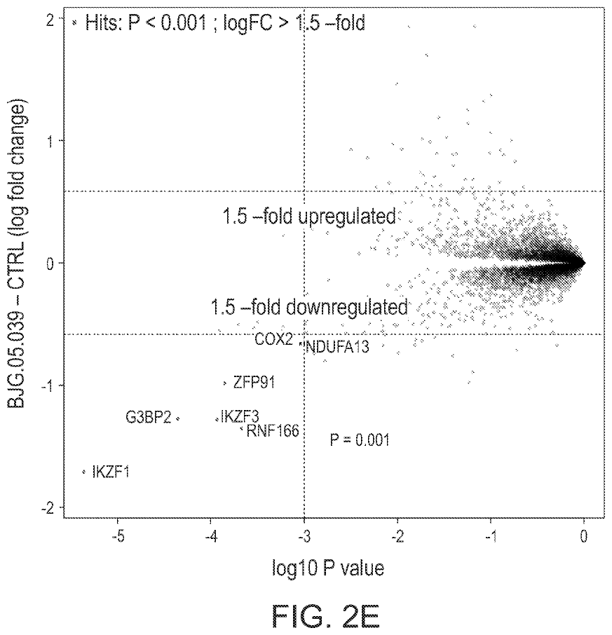

After verifying that BJG-05-039 retained specificity for PAK1, its degradation activity in cells was characterized. First, the PAK1 degrader was evaluated in the breast cancer cell line MCF7 and the ovarian cancer cell line OVCAR3 due to their high expression of both PAK1 and PAK2 and their known dependence on PAK1 (Prudnikova, et al. (2016) Oncogene 35:2178-2185; Shrestha, et al. (2012) Oncogene 31:3397-3408). It was determined that BJG-05-039 induced degradation of PAK1 but not PAK2 in a dose-dependent manner after a 12 hour treatment, with maximal degradation observed at 10 nM (FIG. 2A). At concentrations of 1 μM and greater, diminished PAK1 degradation was observed, consistent with the hook effect, in which independent engagement of PAK1 and CRBN by BJG-05-039 prevents formation of a productive ternary complex (An, et al. (2018) EBioMedicine 36:553-562). PAK1 degradation was not observed in cells treated with the parent molecule, NVS-PAK1-1, or with BJG-05-098, a negative control compound with an N-methylated glutarimide that weakens CRBN binding (FIG. 2A; Brand, et al. (2019) Cell Chem. Biol., 26:300-306). BJG-05-098 did not induce degradation of PAK1 or PAK2, demonstrating that BJG-05-039-induced PAK1 degradation was CRBN dependent. Time course treatment of MCF7 cells with 250 nM BJG-05-039 revealed partial degradation of PAK1 within 4 hours and progressive loss out to 24 hours (FIG. 2B). Co-treatment of cells with BJG-05-039 and bortezomib (a proteasome inhibitor) or MLN4924 (an NAE1 (NEDD8-activating enzyme 1) inhibitor that prevents the neddylation required for the activation of cullin RING ligases, such as Cullin-4-RING E3 ubiquitin ligase (CRL4) CRBN (Soucy, et al. (2009) Clin. Cancer Res., 15:3912-3916)) prevented PAK1 destabilization, indicating that degradation was dependent on the ubiquitin-proteasome system (FIG. 2C).

To better quantify the effect of degrader treatment, HEK293 cells were transfected with an expression vector encoding nano-luciferase (Nluc)-tagged PAK1. The Nluc tag allows for luciferase-based quantitation of protein expression (England, et al. (2016) Bioconjug. Chem., 27:1175-1187). Nluc-PAK1 cells were treated with BJG-05-039 or its N-methylated analog, BJG-05-098, and PAK1 expression was assessed (FIG. 2D). This experiment showed that half-maximal degradation of PAK1 was achieved at low nM concentrations of BJG-05-039, with approximately 70% reduction in PAK1 expression following treatment with 10 nM of BJG-05-039.

To assess degrader selectivity across the proteome, MOLT4 cells were treated with 1 μM BJG-05-039 for 5 hours and global multiplexed mass spectrometry-based proteomic analysis was performed (Donovan, et al. (2018) Elife 7:e38430). As expected for thalidomide/pomalidomide-based degraders, the ring-finger protein RNF166 as well as other known IMiD off target proteins, Ikaros (IKZF1) and Aiolos (IKZF3), were strongly downregulated by BJG-05-039 treatment (FIG. 2E) (Kronke, et al. (2015) Nature 523:183-188; Kronke, et al. (2014) Oncoimmunology 3:e941742). Interestingly, while BJG-05-039 showed potent in vitro inhibition of PAK1 and a ˜50% reduction in PAK1 levels as determined by immunoblot (FIG. 2A), significant downregulation of this kinase was not apparent in the proteomic analysis, possibly due to the hook effect at 1 μM.

BJG-05-039 Exhibits Enhanced Effects on Signaling Compared with NVS-PAK1-1

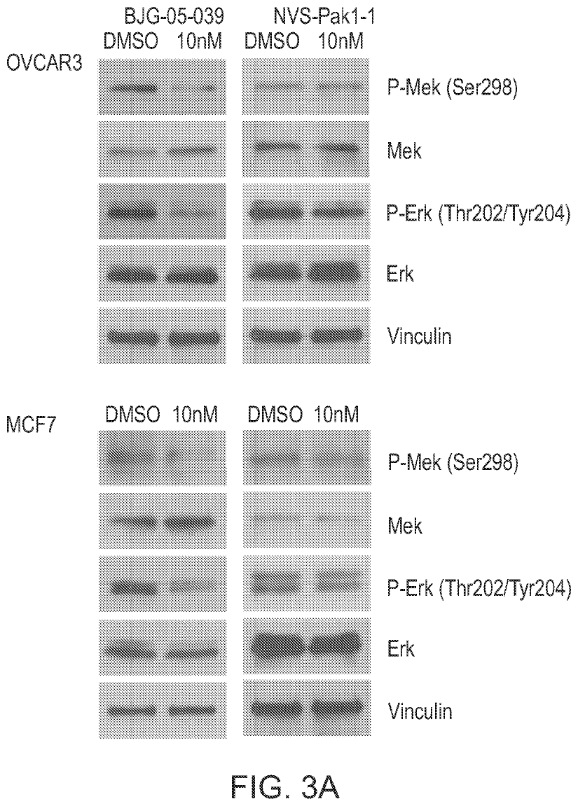

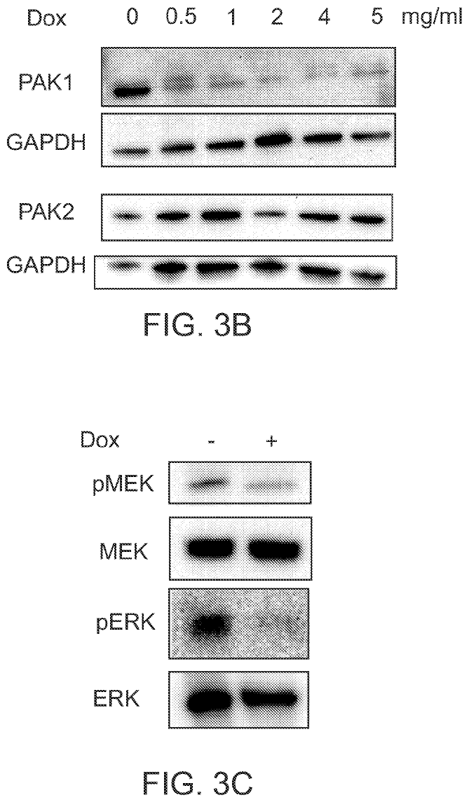

In certain cell lines, PAK1 has well-characterized functions in regulating proliferative signaling. The activity of BJG-05-039 against NVS-PAK1-1 was compared in two such cell lines, OVCAR3 and MCF7. As a readout for anti-PAK activity, phosphorylation of MEK1 at S298 was assessed, a direct target site for Group A PAKs (Coles, et al. (2002) Oncogene 21:2236-2244; Slack-Davis, et al. (2003) J. Cell. Biol., 162:281-291), as well as phosphorylation of the downstream target of MEK, ERK. At a 10 nM dose, BJG-05-039 inhibited phosphorylation of MEK S298, as well as inhibiting phosphorylation of ERK. However, an equivalent dose of NVS-PAK1-1 had no visible impact on phosphorylation status of these proteins (FIG. 3A). Selective knockdown of PAK1 showed similar effects to BJG-05-039 in these cell lines, substantially reducing levels of phospho-MEK and phospho-ERK, whereas knockdown of PAK2 showed little effect (FIG. 3B and FIG. 3C). MCF7 cells were stably transduced with a doxycycline-regulated shRNA against PAK1.

### BJG-05-039 is Effective in Reducing Proliferation in PAK1-Dependent, but not PAK2-Dependent Cell Lines

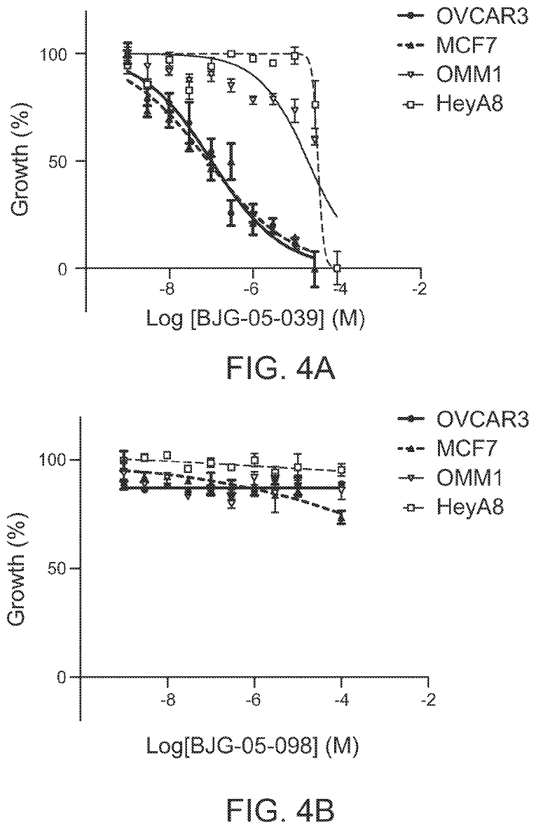

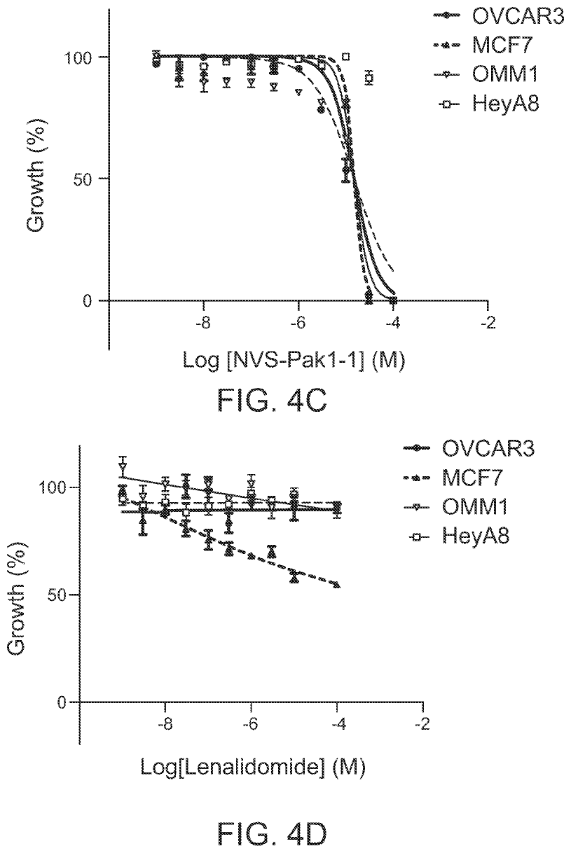

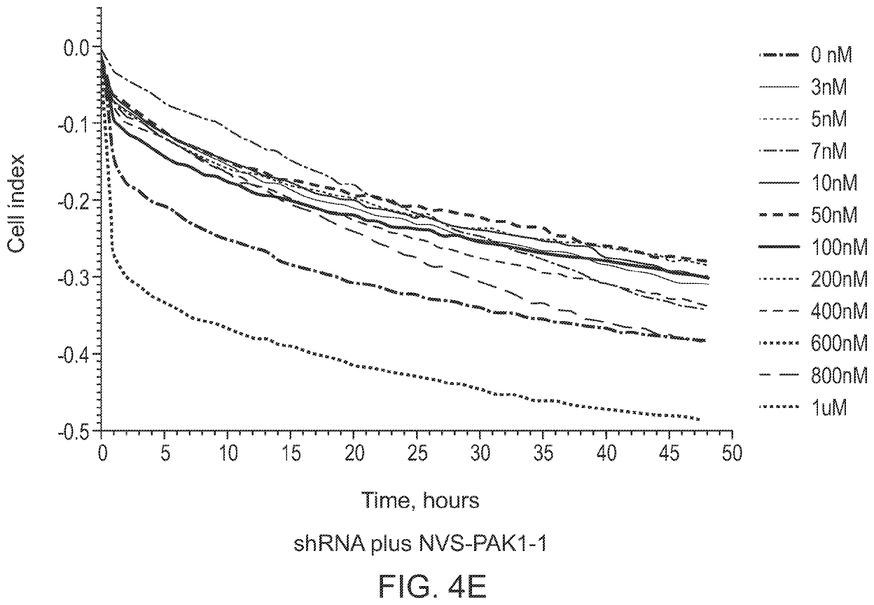

The anti-proliferative effects of PAK1 degradation and inhibition were evaluated. Using the same two cell lines, it was determined that BJG-05-039 was far more potent than NVS-PAK1-1, with EC₅₀ values of 3 and 8.4 nM in MCF7 and OVCAR3 cells, respectively, compared to 11.9 μM and 3.68 μM, respectively, for NVS-PAK1-1 (FIG. 4A-FIG. 4E). These large differences in EC₅₀ values are unlikely to be due to degradation of non-PAK1 targets (e.g., IKZF1 or IKZF3) by BJG-05-039, as lenalidomide lacked significant antiproliferative effects in either cell line (FIG. 4D). Notably, negative control BJG-05-098 did not affect cell proliferation (FIG. 4B). Neither BJG-05-039 or NVS-PAK1-1 significantly affected cell proliferation in OMM1 and HEY-A8 cells, which genome-wide CRISPR-screens indicate are highly dependent on PAK2 (FIG. 4A-FIG. 4E) (depmap.org/portal/gene/PAK2?tab=dependency). In addition, the effects of BJG-05-039 are unlikely to be caused by non-specific degradation of proteins (e.g., IKZF1 or IKZF3), as lenalidomide lacked significant antiproliferative effects in either cell line (FIG. 4D). The EC50 values for BJG-05-039 were: 0.102 μM in OVCAR3, 0.086 μM in MCF7, 20.770 μM in OMM1, and 35.950 μM in Hey A8. The EC50 values for NVS-Pak1-1 were: 8.896 μM in OVCAR3, 11.830 μM in MCF7, 12.020 μM in OMM1, and 33.570 μM in Hey A8. These data indicate that the PAK1 inhibition and degradation by BJG-05-039 is more potent at reducing proliferative signaling in PAK1-dependent cells than what can be achieved by inhibiting catalytic activity alone. Consistent with this view, NVS-PAK1-1 showed much more potent anti-proliferative effects in cells in which PAK1 levels were reduced ˜50% using shRNA (FIG. 4E). These results also support a mechanism of action related to PAK1 inhibition and degradation as opposed to “off-target” degradation effects.

In many cancer cells, Group A PAKs act as signaling hub, coordinating the activation of various central proliferative, survival, and motility pathways (Radu, et al. (2014) Nat. Rev. Cancer 14:13-25). As such, there has been interest in targeting these enzymes with small molecule inhibitors (Liu, et al. (2021) Front Cell. Dev. Biol., 9:641381; Murray, et al. (2010) Proc. Natl. Acad. Sci., 107:9446-9451; Ong, et al. (2015) Breast Cancer Res., 17:59; Ong, et al. (2013) J. Natl. Cancer Inst., 105:606-607; Rudolph, et al. (2015) J. Med. Chem., 58:111-129; Semenova, et al. (2017) Biochem. Soc. Trans., 45:79-88; Senapedis, et al. (2016) Anticancer Agents Med. Chem., 16:75-88). One pan-PAK inhibitor, PF3758309 (Murray, et al. (2010) Proc. Natl. Acad. Sci., 107:9446-9451), was evaluated in a phase 1 clinical trial, but poor pharmacologic properties and excessive toxicity led to its withdrawal. More selective inhibitors, such as FRAX-597, FRAX-1036, and G5555 showed efficacy in cell-based models, in particular, in cells in which the PAK1 gene was amplified or in which RAC1 was a driving oncogene (Chow, et al. (2015) Oncotarget 6:1981-1994; Knippler, et al. (2019) Endocr. Relat. Cancer 26:699-712; Licciulli, et al. (2013) J. Biol. Chem., 288:29105-29114; Ndubaku, et al. (2015) ACS Med. Chem. Lett., 6:1241-1246; Ong, et al. (2015) Breast Cancer Res., 17:59; Prudnikova, et al. (2016) Oncogene 35:2178-2185; Qasim, et al. (2021) Oncogene 40:1176-1190; Semenova, et al. (2017) Oncogene 36:5421-5431; Uribe-Alvarez, et al. (2020) Small GTPases, 12(4):273-281). However, progress in clinical development has been hampered by genetic and pharmacologic evidence suggesting a vital role for PAK2 in cardiovascular function and vascular integrity in adult mammals (Radu, et al. (2015) Mol. Cell. Biol., 35:3990-4005; Rudolph, et al. (2016) J. Med. Chem., 59:5520-5541). Acute cardiotoxicity upon inhibitor treatment or gene loss is thought to be due to a unique role for PAK2 in regulating ER stress and oxidative stress in cardiomyocytes (Binder, et al. (2019) Circulation Res., 124:696-711; Wang, et al. (2019) J. Cardiovasc. Pharmacol., 74:20-29).

While the vast majority of Group A PAK inhibitors equally affect PAK1, -2, and -3, NVS-PAK1-1—an allosteric inhibitor that binds beneath the αC helix rather than in the hinge region of the ATP binding pocket of PAK1—exhibits an approximately 50-fold specificity for PAK1 over PAK2. As with FAK degraders (Cromm, et al. (2018) J. Am. Chem. Soc., 140:17019-17026), PAK1 degraders have the potential to incite more benefit than standard enzymatic inhibitors because removal of PAK1 would not only reduce its kinase enzymatic activity, but also its scaffolding function, both of which mediate signaling activity. For example, PAK1 has been shown to be required for AKT activation (Chow, et al. (2012) Cancer Res., 72:5966-5975; Higuchi, et al. (2008) Nat. Cell. Biol., 10:1356-1364), but these effects map to the N-terminus of PAK1 and appear to be independent of kinase activity (Higuchi, et al. (2008) Nat. Cell. Biol., 10:1356-1364). Degraders also offer the potential for prolonged efficacy, which is driven by target half-life, and this is an important consideration given the short half-life of NVS-PAK1-1 (Hawley, et al. (2021) Human Mol. Genet., 30(17):1607-1617; Karpov, et al. (2015) ACS Med. Chem. Lett., 6:776-781). While adding the linker and thalidomide/pomalidomide moiety to NVS-PAK-1 reduced the potency of BJG-05-039 as a PAK1 catalytic inhibitor, it also increased its selectivity over PAK2 more than ten-fold (Table 1). Given the necessity for isoform selective targeting, this improved selectivity for PAK1 will reduce the toxicity of this molecule relative to the parental NVS-PAK1-1.

In the absence of drug efflux inhibitors, a 70% loss of the PAK1 protein using BJG-05-039 was observed. The combination of catalytic inhibition and lowered protein expression had dramatically improved inhibitory effects on signaling and proliferation in PAK1-dependent cell lines (FIG. 3A and FIG. 4A). In this respect, the degrader may be mimicking the effects of genetic manipulations such as RNAi-mediated gene knock down. This property can be used, in conjunction with the parental inhibitor NVS-PAK1-1, to tease apart kinase vs. scaffolding effects of PAK1 in cells. In fact, the data indicates that reducing the total level of PAK1 expression synergizes with catalytic expression, as evidenced by the greatly increased potency of the non-degrader NVS-PAK-1 when used in conjunction with partial knockdown of PAK1 with shRNA (FIG. 4C). Thus, in addition to its potential role in reigniting interest in clinical development of PAK inhibitors, the degrader compound described herein provides a useful tool compound for signaling analysis.

Given their role in regulating the ERK, AKT, and b-catenin pathways, Group A PAKs have been considered as potential therapeutic targets in cancer. However, the PAK2 isoform plays a key role in normal cardiovascular function in adult mammals, and this factor has impeded further preclinical development of anti-PAK agents. Selective PAK2-sparing molecules present a path forward. Given that PAK1 has significant scaffolding activity in addition to its catalytic activity, a degrader based on NVS-PAK1-1 provides considerable benefits while avoiding toxicities associated with PAK2 inhibition. Herein, it is shown that an optimized degrader, BJG-05-039, promoted rapid and selective inhibition and degradation of PAK1 but not PAK2, and had more potent anti-proliferative effects than NVS-PAK-1. Given that many cancer cell types display elevated PAK1 expression and activity, and knockdown of PAK1 has been shown to have considerable anti-proliferative and/or anti-survival effects in these settings, isoform-specific inhibition and degradation of PAK1 provides an effective measure for treating cancer.

All patent publications and non-patent publications are indicative of the level of skill of those skilled in the art to which this invention pertains. All these publications (including any specific portions thereof that are referenced) are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound of Formula (I): 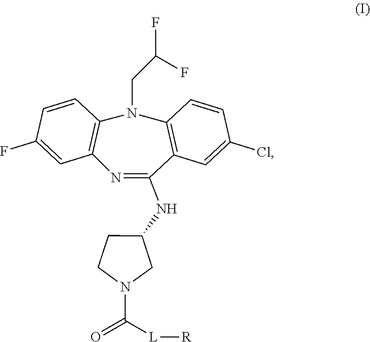
wherein L is a linker and R is a degron,
the linker is a chemical moiety that covalently attaches the carbonyl carbon to the degron; and
the degron is a ligand for an E3 ubiquitin ligase,
or a pharmaceutically acceptable salt or stereoisomer thereof.

2. The compound of claim 1, wherein the compound is of Formula (II): 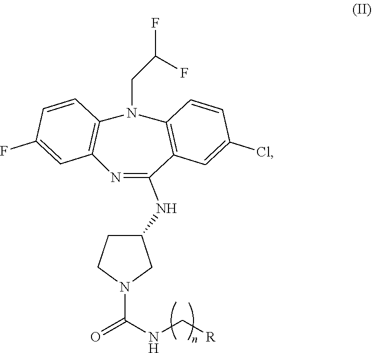
wherein R is a degron and n=1 to 15,
or a pharmaceutically acceptable salt or stereoisomer thereof.

3. The compound of claim 1, wherein the E3 ubiquitin ligase is the cereblon (CRBN) E3 ubiquitin ligase.

4. The compound of claim 3, wherein the degron is of formula D1: 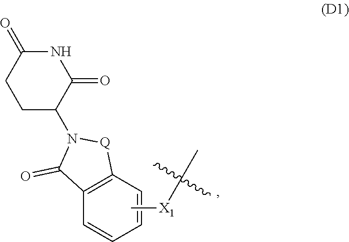
wherein Q is CH₂ or C(O); and
X₁ is a bond, CH₂, O, NH, or C≡C.

5. The compound of claim 3, wherein said degron is pomalidomide, 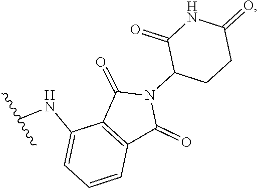 [SMILES: *Nc1cccc2c1C(=O)N(C1CCC(=O)NC1=O)C2=O]
or analog thereof.

6. The compound of claim 1, wherein the E3 ubiquitin ligase is the Von Hippel-Lindau (VHL) E3 ubiquitin ligase.

7. The compound of claim 6, wherein the degron is represented by any one of the structures (D2-a) to (D2-f): 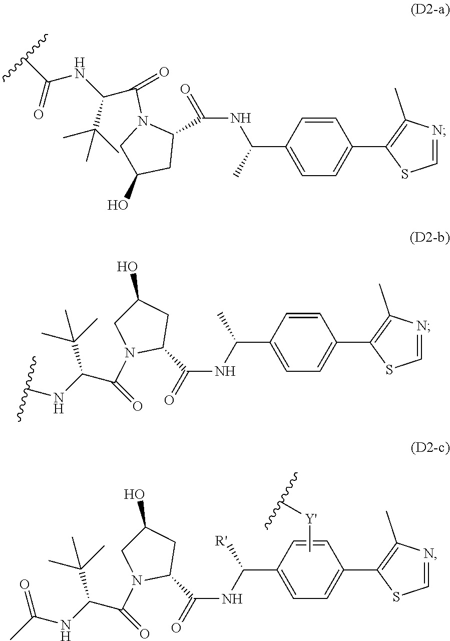
wherein Y′ is a bond, NH, O or CH₂ and R′ is H or methyl; 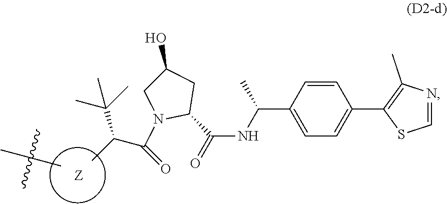
wherein Z is a C₅-C₆ carbocycle or a C₅-C₆ heterocyclic group; 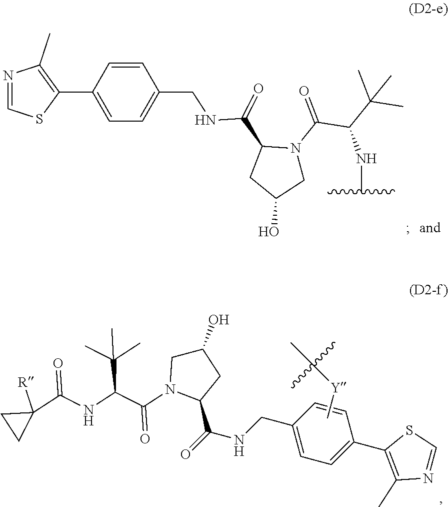
wherein Y″ is a bond, NH, O or CH₂ and R″ is F or CN, or a stereoisomer thereof.

8. The compound of claim 6, wherein said degron is 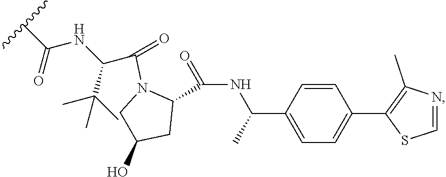 [SMILES: *C(=O)N[C@H](C(=O)N1C[C@H](O)C[C@H]1C(=O)N[C@@H](C)c1ccc(-c2scnc2C)cc1)C(C)(C)C]
or stereoisomer thereof.

9. The compound of claim 1, wherein said degron is lenalidomide, 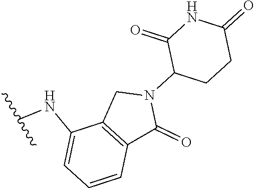 [SMILES: *Nc1cccc2c1CN(C1CCC(=O)NC1=O)C2=O]
or analog thereof.

10. The compound of claim 1, wherein the linker is a bond or an alkylene chain which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

11. The compound of claim 1, wherein the linker is a polyethylene glycol (PEG) chain which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

12. The compound of claim 1, wherein L is a hydrocarbon, alkyl, or alkenyl.

13. The compound of claim 1, wherein L is

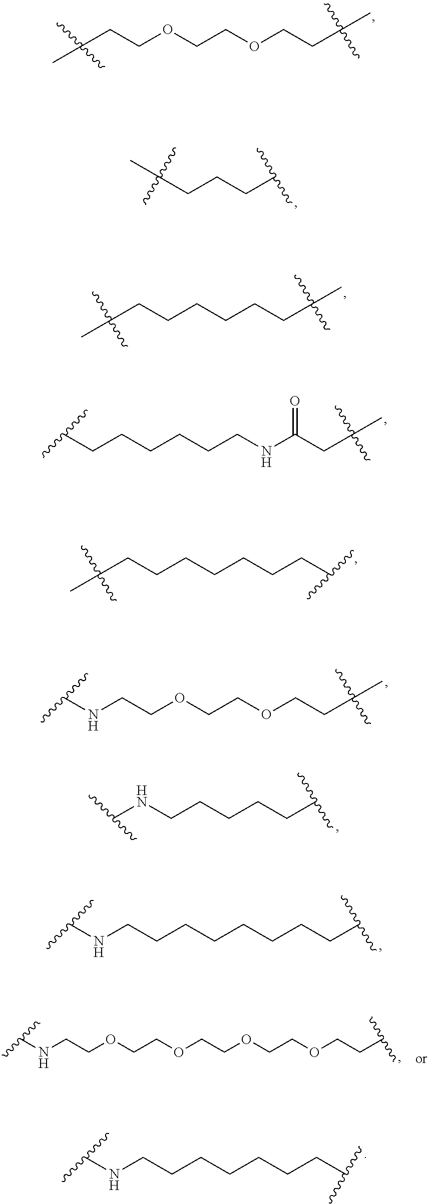

14. The compound of claim 1, which is 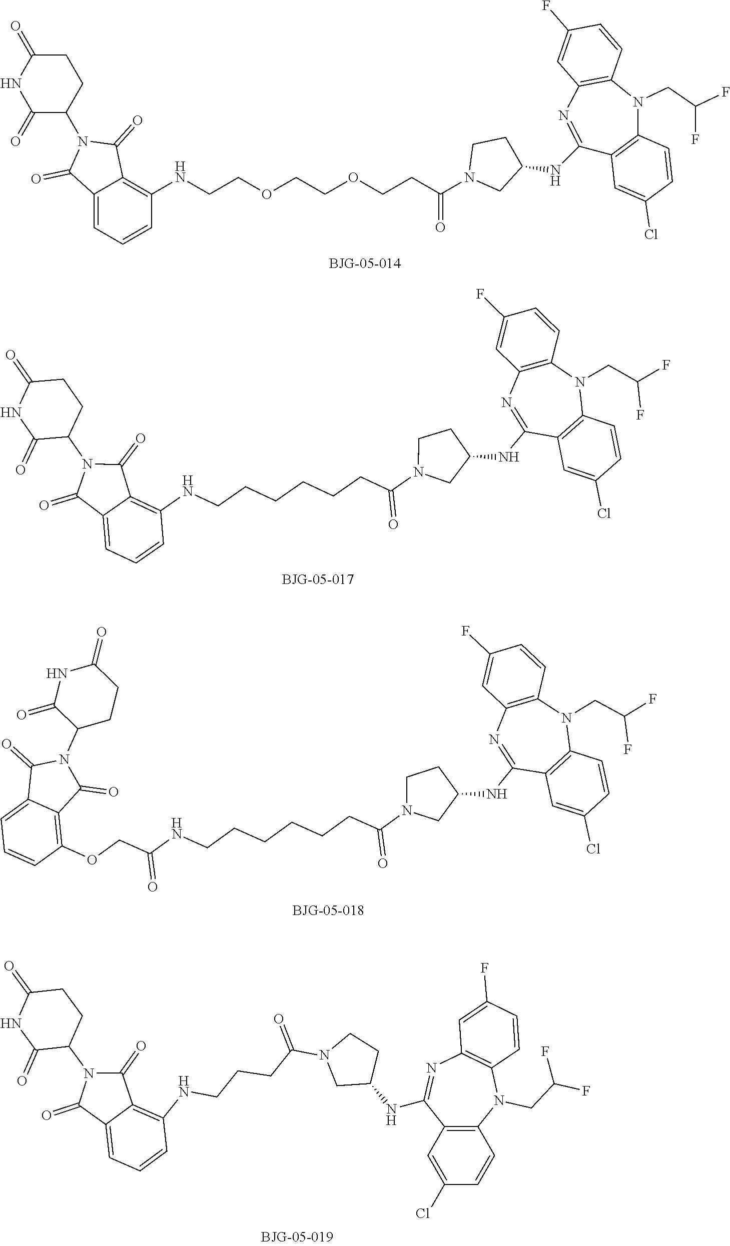 [SMILES: O=C1CCC(N2C(=O)c3cccc(NCCOCCOCCC(=O)N4CC[C@H](NC5=Nc6cc(F)ccc6N(CC(F)F)c6ccc(Cl)cc65)C4)c3C2=O)C(=O)N1 ; O=C1CCC(N2C(=O)c3cccc(NCCCCCCC(=O)N4CC[C@H](NC5=Nc6cc(F)ccc6N(CC(F)F)c6ccc(Cl)cc65)C4)c3C2=O)C(=O)N1 ; O=C(COc1cccc2c1C(=O)N(C1CCC(=O)NC1=O)C2=O)NCCCCCCC(=O)N1CC[C@H](NC2=Nc3cc(F)ccc3N(CC(F)F)c3ccc(Cl)cc32)C1 ; O=C1CCC(N2C(=O)c3cccc(NCCCC(=O)N4CC[C@H](NC5=Nc6cc(F)ccc6N(CC(F)F)c6ccc(Cl)cc65)C4)c3C2=O)C(=O)N1] 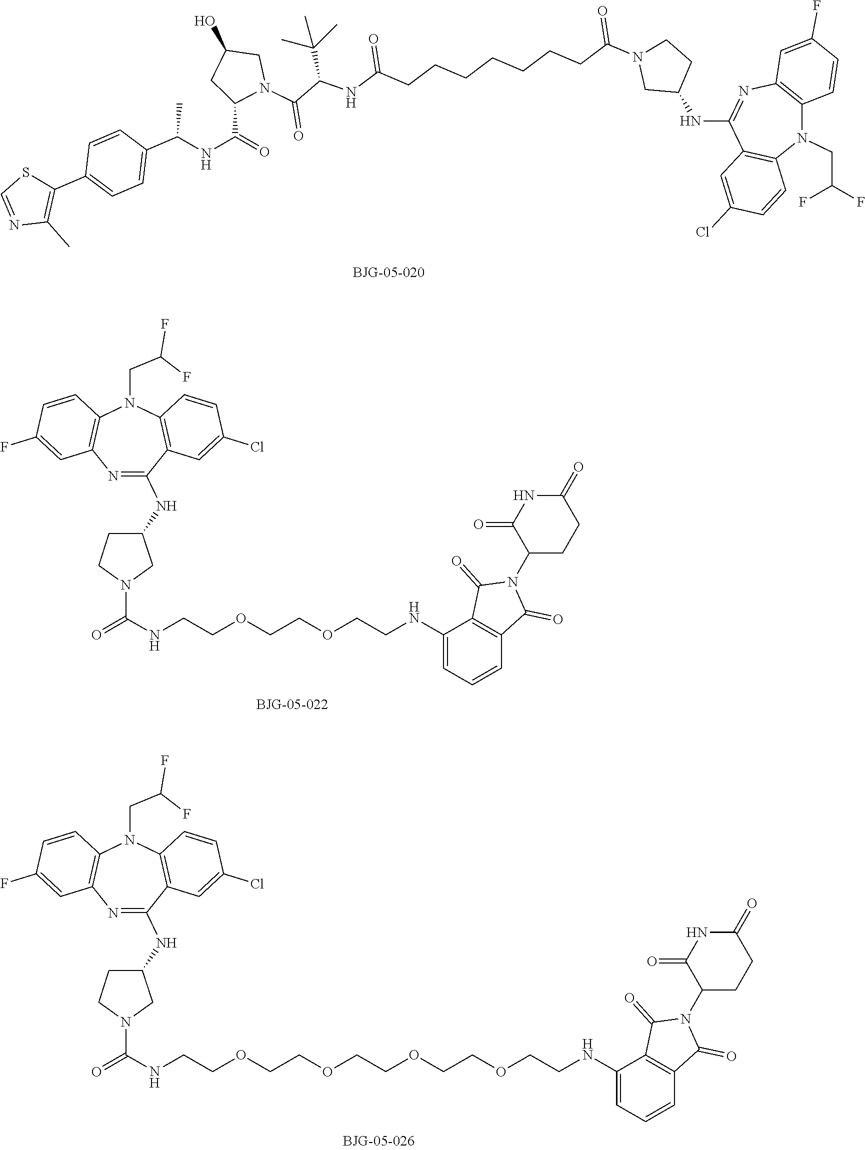 [SMILES: Cc1ncsc1-c1ccc([C@H](C)NC(=O)[C@@H]2C[C@@H](O)CN2C(=O)[C@@H](NC(=O)CCCCCCCC(=O)N2CC[C@H](NC3=Nc4cc(F)ccc4N(CC(F)F)c4ccc(Cl)cc43)C2)C(C)(C)C)cc1 ; O=C1CCC(N2C(=O)c3cccc(NCCOCCOCCNC(=O)N4CC[C@H](NC5=Nc6cc(F)ccc6N(CC(F)F)c6ccc(Cl)cc65)C4)c3C2=O)C(=O)N1 ; O=C1CCC(N2C(=O)c3cccc(NCCOCCOCCOCCOCCNC(=O)N4CC[C@H](NC5=Nc6cc(F)ccc6N(CC(F)F)c6ccc(Cl)cc65)C4)c3C2=O)C(=O)N1] 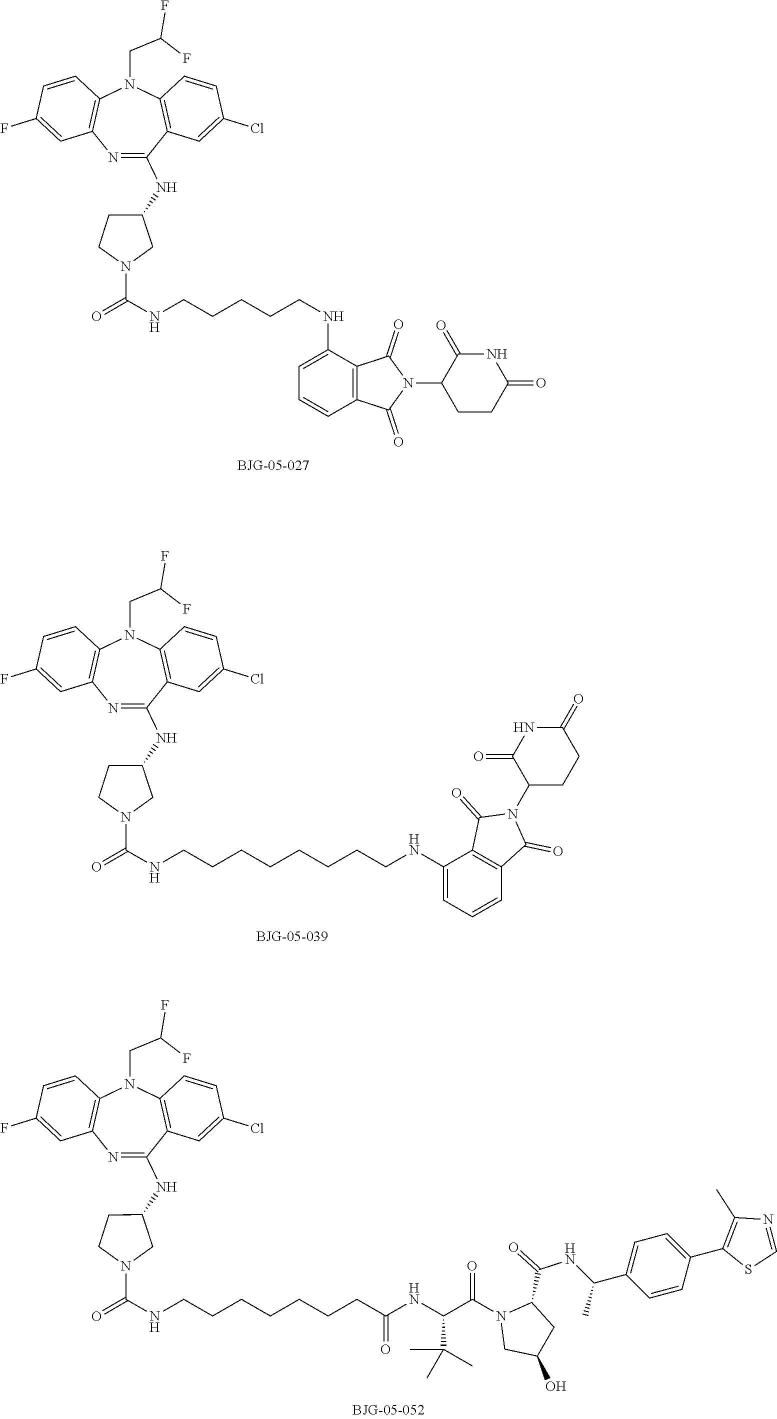 [SMILES: O=C1CCC(N2C(=O)c3cccc(NCCCCCNC(=O)N4CC[C@H](NC5=Nc6cc(F)ccc6N(CC(F)F)c6ccc(Cl)cc65)C4)c3C2=O)C(=O)N1 ; O=C1CCC(N2C(=O)c3cccc(NCCCCCCCCNC(=O)N4CC[C@H](NC5=Nc6cc(F)ccc6N(CC(F)F)c6ccc(Cl)cc65)C4)c3C2=O)C(=O)N1 ; Cc1ncsc1-c1ccc([C@H](C)NC(=O)[C@@H]2C[C@@H](O)CN2C(=O)[C@@H](NC(=O)CCCCCCCNC(=O)N2CC[C@H](NC3=Nc4cc(F)ccc4N(CC(F)F)c4ccc(Cl)cc43)C2)C(C)(C)C)cc1] 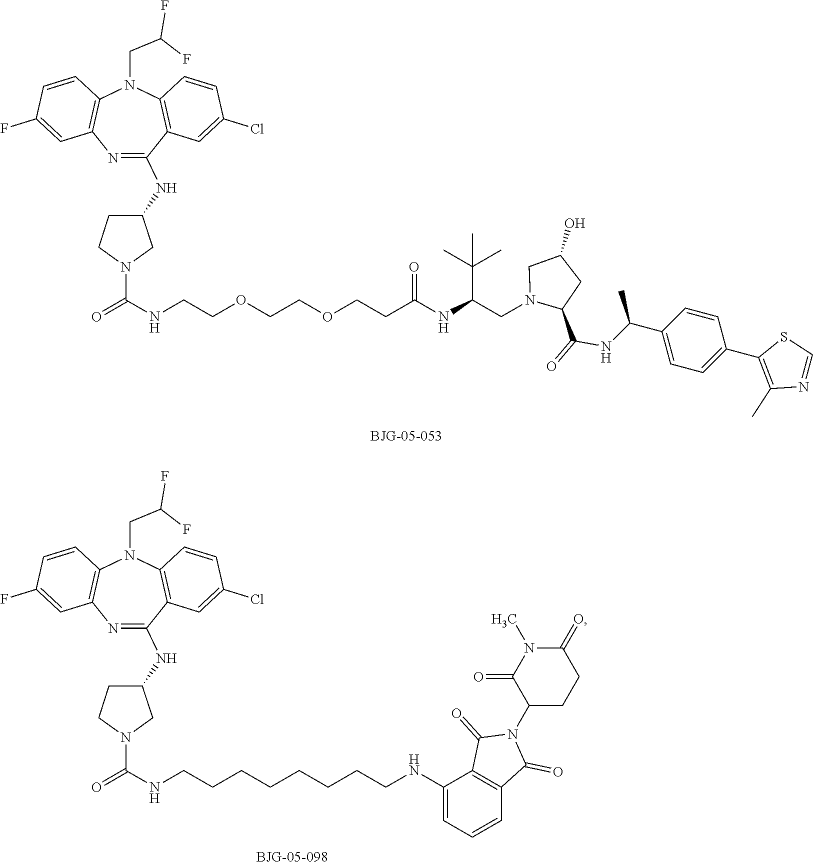 [SMILES: Cc1ncsc1-c1ccc([C@H](C)NC(=O)[C@@H]2C[C@@H](O)CN2C[C@@H](NC(=O)CCOCCOCCNC(=O)N2CC[C@H](NC3=Nc4cc(F)ccc4N(CC(F)F)c4ccc(Cl)cc43)C2)C(C)(C)C)cc1 ; CN1C(=O)CCC(N2C(=O)c3cccc(NCCCCCCCCNC(=O)N4CC[C@H](NC5=Nc6cc(F)ccc6N(CC(F)F)c6ccc(Cl)cc65)C4)c3C2=O)C1=O]
or a pharmaceutically acceptable salt or stereoisomer thereof.

15. The compound of claim 14, which is 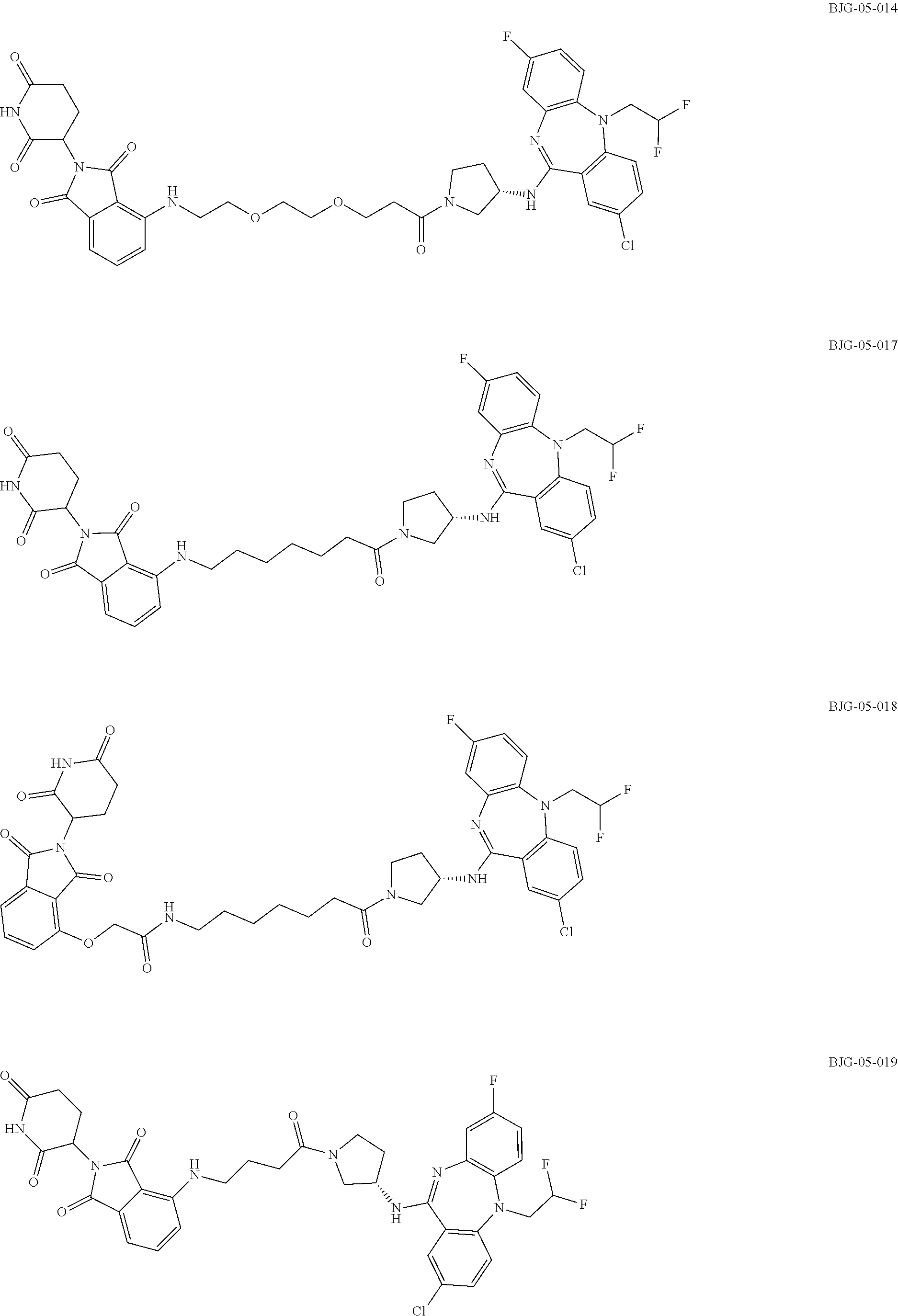 [SMILES: O=C1CCC(N2C(=O)c3cccc(NCCOCCOCCC(=O)N4CC[C@H](NC5=Nc6cc(F)ccc6N(CC(F)F)c6ccc(Cl)cc65)C4)c3C2=O)C(=O)N1 ; O=C1CCC(N2C(=O)c3cccc(NCCCCCCC(=O)N4CC[C@H](NC5=Nc6cc(F)ccc6N(CC(F)F)c6ccc(Cl)cc65)C4)c3C2=O)C(=O)N1 ; O=C(COc1cccc2c1C(=O)N(C1CCC(=O)NC1=O)C2=O)NCCCCCCC(=O)N1CC[C@H](NC2=Nc3cc(F)ccc3N(CC(F)F)c3ccc(Cl)cc32)C1 ; O=C1CCC(N2C(=O)c3cccc(NCCCC(=O)N4CC[C@H](NC5=Nc6cc(F)ccc6N(CC(F)F)c6ccc(Cl)cc65)C4)c3C2=O)C(=O)N1] 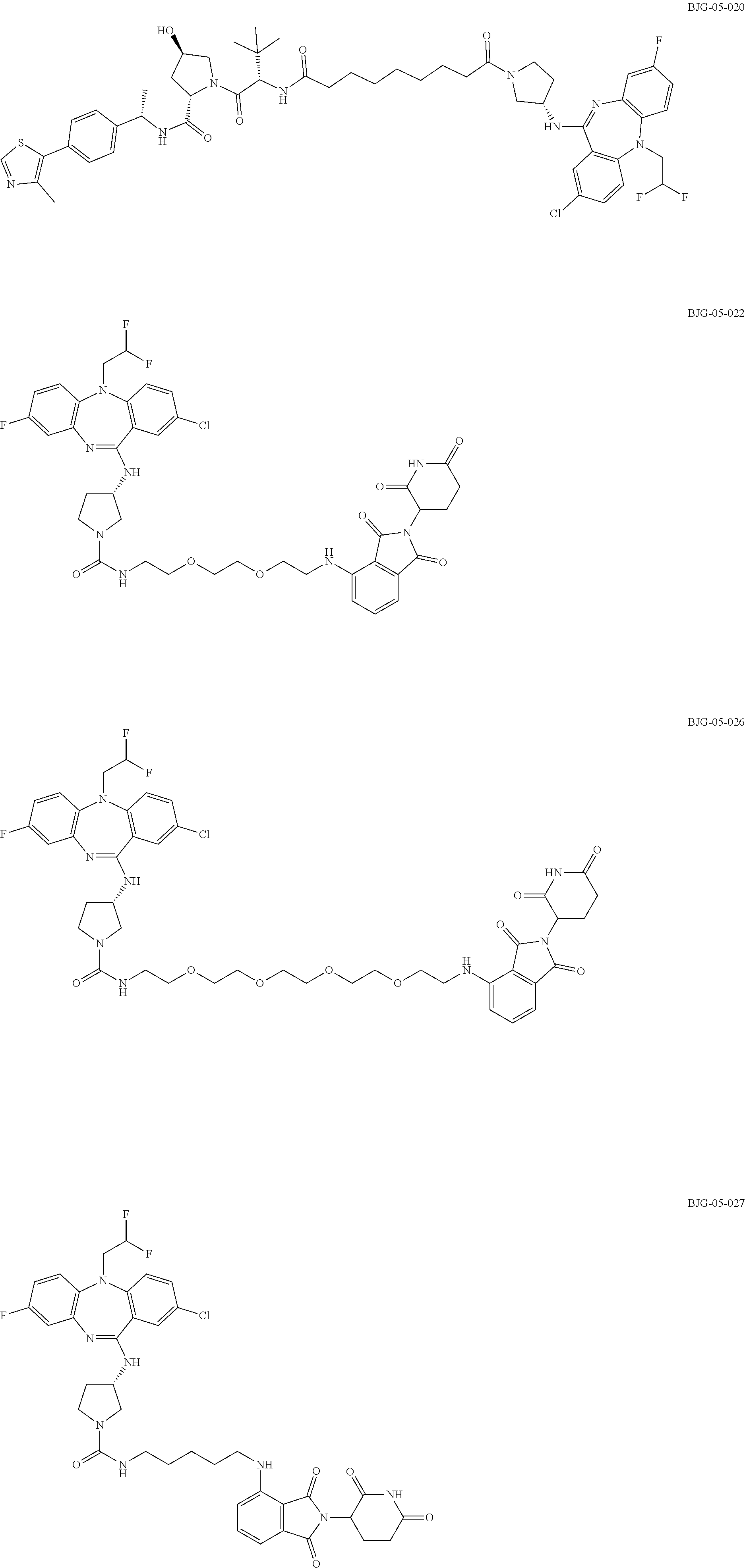 [SMILES: Cc1ncsc1-c1ccc([C@H](C)NC(=O)[C@@H]2C[C@@H](O)CN2C(=O)[C@@H](NC(=O)CCCCCCCC(=O)N2CC[C@H](NC3=Nc4cc(F)ccc4N(CC(F)F)c4ccc(Cl)cc43)C2)C(C)(C)C)cc1 ; O=C1CCC(N2C(=O)c3cccc(NCCOCCOCCNC(=O)N4CC[C@H](NC5=Nc6cc(F)ccc6N(CC(F)F)c6ccc(Cl)cc65)C4)c3C2=O)C(=O)N1 ; O=C1CCC(N2C(=O)c3cccc(NCCOCCOCCOCCOCCNC(=O)N4CC[C@H](NC5=Nc6cc(F)ccc6N(CC(F)F)c6ccc(Cl)cc65)C4)c3C2=O)C(=O)N1 ; O=C1CCC(N2C(=O)c3cccc(NCCCCCNC(=O)N4CC[C@H](NC5=Nc6cc(F)ccc6N(CC(F)F)c6ccc(Cl)cc65)C4)c3C2=O)C(=O)N1] 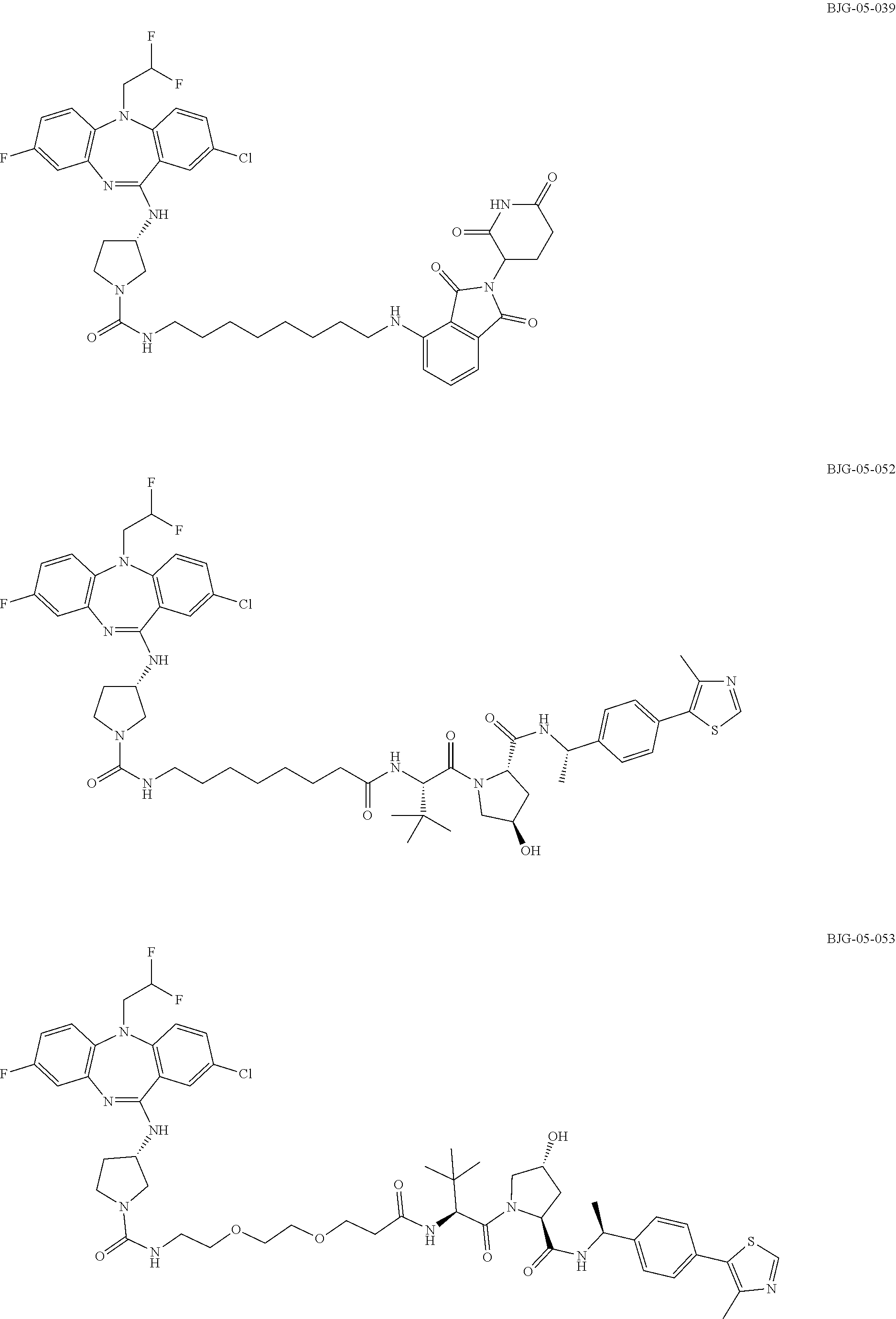 [SMILES: O=C1CCC(N2C(=O)c3cccc(NCCCCCCCCNC(=O)N4CC[C@H](NC5=Nc6cc(F)ccc6N(CC(F)F)c6ccc(Cl)cc65)C4)c3C2=O)C(=O)N1 ; Cc1ncsc1-c1ccc([C@H](C)NC(=O)[C@@H]2C[C@@H](O)CN2C(=O)[C@@H](NC(=O)CCCCCCCNC(=O)N2CC[C@H](NC3=Nc4cc(F)ccc4N(CC(F)F)c4ccc(Cl)cc43)C2)C(C)(C)C)cc1 ; Cc1ncsc1-c1ccc([C@H](C)NC(=O)[C@@H]2C[C@@H](O)CN2C(=O)[C@@H](NC(=O)CCOCCOCCNC(=O)N2CC[C@H](NC3=Nc4cc(F)ccc4N(CC(F)F)c4ccc(Cl)cc43)C2)C(C)(C)C)cc1]
or a pharmaceutically acceptable salt or stereoisomer thereof.

16. The compound of claim 1, which is 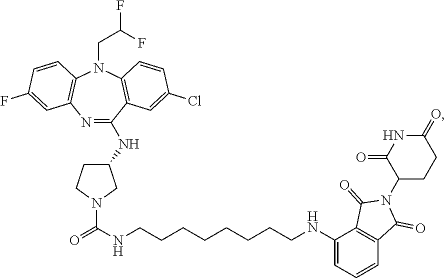 [SMILES: O=C1CCC(N2C(=O)c3cccc(NCCCCCCCCNC(=O)N4CC[C@H](NC5=Nc6cc(F)ccc6N(CC(F)F)c6ccc(Cl)cc65)C4)c3C2=O)C(=O)N1]
or a pharmaceutically acceptable salt or stereoisomer thereof.

17. A compound of Formula (III): 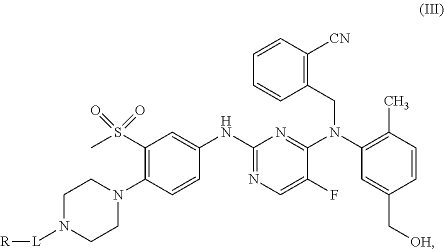
wherein L is a linker and R is a degron,
the linker is a chemical moiety that covalently attaches the nitrogen to the degron; and
the degron is a ligand for an E3 ubiquitin ligase,
or a pharmaceutically acceptable salt or stereoisomer thereof.

18. The compound of claim 17, wherein the E3 ubiquitin ligase is the cereblon (CRBN) E3 ubiquitin ligase.

19. The compound of claim 18, wherein the degron is of formula DL: 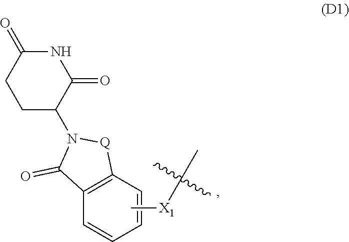
wherein Q is CH₂ or C(O); and
X₁ is a bond, CH₂, O, NH, or C≡C.

20. The compound of claim 18, wherein said degron is pomalidomide, 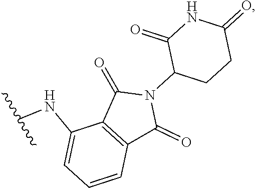 [SMILES: *Nc1cccc2c1C(=O)N(C1CCC(=O)NC1=O)C2=O]
or analog thereof.

21. The compound of claim 17, wherein the linker is a polyethylene glycol (PEG) chain which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

22. The compound of claim 17, which is 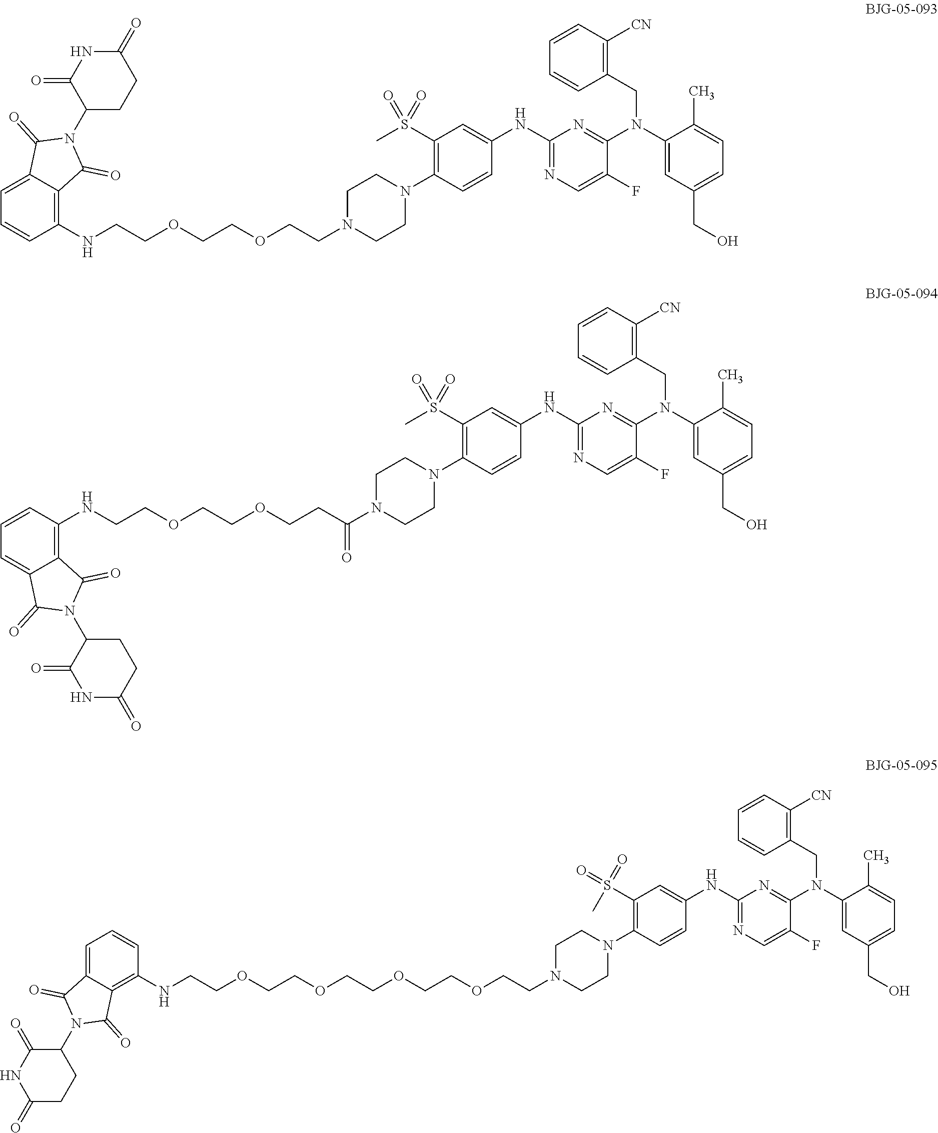 [SMILES: Cc1ccc(CO)cc1N(Cc1ccccc1C#N)c1nc(Nc2ccc(N3CCN(CCOCCOCCNc4cccc5c4C(=O)N(C4CCC(=O)NC4=O)C5=O)CC3)c(S(C)(=O)=O)c2)ncc1F ; Cc1ccc(CO)cc1N(Cc1ccccc1C#N)c1nc(Nc2ccc(N3CCN(C(=O)CCOCCOCCNc4cccc5c4C(=O)N(C4CCC(=O)NC4=O)C5=O)CC3)c(S(C)(=O)=O)c2)ncc1F ; Cc1ccc(CO)cc1N(Cc1ccccc1C#N)c1nc(Nc2ccc(N3CCN(CCOCCOCCOCCOCCNc4cccc5c4C(=O)N(C4CCC(=O)NC4=O)C5=O)CC3)c(S(C)(=O)=O)c2)ncc1F]
or a pharmaceutically acceptable salt or stereoisomer thereof.

23. A composition comprising a compound of claim 1 and a pharmaceutically acceptable carrier.

24. A method of treating, inhibiting, and/or preventing a cancer or other PAK1 associated disease or disorder in a subject in need thereof, said method comprising administering a compound of claim 1 to the subject.

25. The method of claim 24, said method further comprising administering another therapy to said subject.

26. The method of claim 24, wherein said PAK1 associated disease or disorder is neurofibromatosis type 1 (NF1) or neurofibromatosis type 2 (NF2).
